(19) United States
(12) Patent Application Publication
(10) Pub. No.: US 2025/0043259 A1
(43) Pub. Date: Feb. 6, 2025

(54) ENDONUCLEASES THAT SELECTIVELY CLEAVE SINGLE-STRANDED NUCLEIC ACIDS AND USES THEREOF

(71) Applicant: INSTITUT NATIONAL DE LA RECHERCHE SCIENTIFIQUE, Québec (CA)

(72) Inventors: Frédéric VEYRIER, Montréal (CA); Martin CHENAL, Montréal (CA)

(21) Appl. No.: 18/709,349

(22) PCT Filed: Nov. 11, 2022
(86) PCT No.: PCT/CA2022/051668

§ 371 Date: May 10, 2024

Related U.S. Application Data
(60) Provisional application No. 63/263,896, filed on Nov. 11, 2021.

(51) Int. Cl. C12N 9/22

(52) U.S. Cl. CPC C12N 9/22; C12Y 301/21001

(57) ABSTRACT

The present application relates to endonucleases specific for single-stranded nucleic acid molecules. These endonuclease s having a length of 60 to 150 amino acids or less and comprises a single GIY-YIG domain. The present application also relates to compositions and cells comprising such endonucleases as well as to methods of cleaving single-stranded nucleic acid molecules comprising a nucleotide sequence recognized by the endonucleases.

### CROSS REFERENCE TO RELATED APPLICATIONS

The present application claims the benefit of U.S. provisional patent application Ser. No. 63/263,896, filed on Nov. 11, 2021, which is incorporated herein by reference in its entirety.

### TECHNICAL FIELD

The present disclosure generally relates to the fields of enzymology and molecular biology, and more particularly to endonucleases.

### BACKGROUND ART

Endonucleases are enzymes that cleave the phosphodiester bond within a polynucleotide chain. Two types of endonucleases are restriction nucleases and homing endonucleases.

Restriction endonucleases are enzymes that recognize a specific nucleotide sequence in a double-stranded nucleic acid called a restriction site. Upon binding to the restriction site, the restriction endonucleases cleave within or near the restriction site. These enzymes are routinely used for DNA modification in laboratories, such as for genetic engineering and molecular cloning. For example, they are used to assist insertion of genes into plasmid vectors, to distinguish gene alleles by specifically recognizing single base changes in DNA known as single-nucleotide polymorphisms (SNPs), to digest genomic DNA for gene analysis, and to insert nucleic acid molecules within the genome of an organism. Most of the known restriction enzymes recognize a restriction site, typically comprising from 4 to 8 nucleotides that are often palindromic, within a double-stranded DNA molecule, and produce a double-stranded cut in the DNA.

Homing endonucleases are double-stranded DNases that have large, asymmetric recognition sites (12-40 base pairs). However, unlike restriction endonucleases, homing endonucleases tolerate some sequence degeneracy within their recognition sequence, which means that single base changes do not abolish cleavage but reduce its efficiency to variable extents. As a result, their observed sequence specificity is typically in the range of 10-12 base pairs.

In contrast to restriction and homing endonucleases that cleaves double-stranded nucleic acids at specific sites, endonucleases that cleave single-stranded nucleic acid molecules are usually non-specific, i.e., they do not recognize a specific sequence (e.g., restriction site) within the single-stranded nucleic acid molecules but rather cleave at various sequences to degrade the nucleic acid molecules in several fragments.

Thus, there is a need for the identification of novel endonucleases that are able to recognize and cleave at specific sequences within single-stranded nucleic acid molecules such as single-stranded DNA molecules.

The present description refers to a number of documents, the content of which is herein incorporated by reference in their entirety.

### SUMMARY

The present disclosure provides the following items 1 to 73:

1. An isolated endonuclease specific for single-stranded desoxyribonucleic acid molecules, the isolated endonuclease having a length of 60 to 150 amino acids or less and comprising a single GIY-YIG domain.

2. The isolated endonuclease of item 1, having a length of 70 to 130 amino acids.

3. The isolated endonuclease of item 1, having a length of 80 to 120 amino acids.

4. The isolated endonuclease of any one of items 1 to 3, wherein the GIY-YIG domain is of the following sequence (I):

X1-X2-X3-B1-X4-X5-X6-B2-X7-B3-X8 (I)
wherein
   X1 is any amino acid;
   X2 is any amino acid;
   X3 is Y or H;
   B1 is a sequence of 8 to 12 amino acids;
   X4 is Y or H;
   X5 is any amino acid.
   X6 is G or D;
   B2 is a sequence of 6 to 15 amino acids;
   X7 is R or T;
   B3 is a sequence of 30 to 40 amino acids; and
   X8 is E.

5. The isolated endonuclease of item 4, wherein X1 is Y, W, V, A, F, I, C, H, R, T or S.

6. The isolated endonuclease of item 5, wherein X1 is Y.

7. The isolated endonuclease of any one of items 4 to 6, wherein X2 is V, I, L, T, A or F.

8. The isolated endonuclease of item 7, wherein X2 is V.

9. The isolated endonuclease of any one of items 4 to 8, wherein X3 is Y.

10. The isolated endonuclease of any one of items 4 to 9, wherein B1 is a sequence of 8 to 10 amino acids.

11. The isolated endonuclease of any one of items 4 to 10, wherein X4 is Y.

12. The isolated endonuclease of any one of items 4 to 11, wherein X5 is I, L, V, T, A, C, or K.

13. The isolated endonuclease of item 12, wherein X5 is I, T or V.

14. The isolated endonuclease of item 13, wherein X5 is I.

15. The isolated endonuclease of any one of items 4 to 14, wherein X6 is G.

16. The isolated endonuclease of any one of items 4 to 15, wherein B2 is a sequence of 6 to 10 amino acids.

17. The isolated endonuclease of item 16, wherein B2 is a sequence of 6 to 8 amino acids.

18. The isolated endonuclease of any one of items 4 to 17, wherein X7 is R.

19. The isolated endonuclease of any one of items 4 to 18, wherein B3 is a sequence of 35 to 40 amino acids.

20. The isolated endonuclease of any one of items 4 to 19, wherein X8 is E.

21. The isolated endonuclease of any one of items 4 to 20, wherein the GIY-YIG domain is of the following sequence (II):

X1-X2-X3-B1-X4-X5-X6-B2-X7-B4-X9-B5-X8 (II)
wherein

X1, X2, X3, B1, X4, X5, X6, B2, X7 and X8 are as defined in items 4 to 20;
B4 is a sequence of 1 to 5 amino acids;
X9 is H, Q or Y; and
B5 is a sequence of 30 to 38 amino acids.

22. The isolated endonuclease of item 21, wherein B4 is a sequence of 2 to 4 amino acids.

23. The isolated endonuclease of item 21 or 22, wherein X9 is H.

24 The isolated endonuclease of any one of items 21 to 23, wherein B5 is a sequence of 30 to 35 amino acids.

25. The isolated endonuclease of item 24, wherein B5 is a sequence of 31 to 33 amino acids.

26. The isolated endonuclease of any one of items 4 to 25, wherein the GIY-YIG domain is of the following sequence (III):

X1-X2-X3-B1-X4-X5-X6-B2-X7-B4-X9-B5-X8-B6-X10 (III)
wherein
X1, X2, X3, B1, X4, X5, X6, B2, X7, B4, X9, B5 and X8 are as defined in items 4 to 20;
B6 is a sequence of 15 to 20 amino acids; and
X10 is N or K.

27. The isolated endonuclease of item 26, wherein B6 is a sequence of 16 to 19 amino acids.

28. The isolated endonuclease of item 26, wherein X10 is N.

29. The isolated endonuclease of any one of items 1 to 28, comprising an amino acid sequence having at least 50% similarity or identity with any one of the sequences set forth in SEQ ID NOs: 2-2891.

30. The isolated endonuclease of any one of items 1 to 28, wherein the isolated endonuclease comprises an amino acid sequence having at least 60% similarity or identity with any one of the sequences set forth in SEQ ID NOs: 2-2891.

31. The isolated endonuclease of any one of items 1 to 28, wherein the isolated endonuclease comprises an amino acid sequence having at least 70% similarity or identity with any one of the sequences set forth in SEQ ID NOs: 2-2891.

32. The isolated endonuclease of any one of items 1 to 28, wherein the isolated endonuclease comprises an amino acid sequence having at least 80% similarity or identity with any one of the sequences set forth in SEQ ID NOs: 2-2891.

33. The isolated endonuclease of any one of items 1 to 28, wherein the isolated endonuclease comprises an amino acid sequence having at least 90% similarity or identity with any one of the sequences set forth in SEQ ID NOs: 2-2891.

34. The isolated endonuclease of any one of items 1 to 28, wherein the isolated endonuclease comprises the amino acid sequence of any one of the sequences set forth in SEQ ID NOs: 2-2891.

35. A composition comprising (i) the isolated endonuclease of any one of items 1 to 34, and (ii) an aqueous saline solution or buffer.

36. The composition of item 35, wherein the aqueous saline solution or buffer comprises a metal.

37. The composition of item 36, wherein the metal is in the form of a metal salt.

38. The composition of item 36 or 37, wherein the metal is magnesium, manganese or nickel.

39. The composition of item 38, wherein the metal is magnesium.

40. The composition of item 39, wherein the composition comprises magnesium chloride (MgCl₂).

41. The composition of any one of items 35 to 40, wherein the single-stranded nucleic acid molecule is a single-stranded DNA molecule.

42. The composition of any one of items 35 to 41, wherein the single-stranded nucleic acid molecule comprises a nucleotide sequence having at least 50% sequence identity with the sequence:

GTCATTCCCNNNNNNNNGGGAATC
or
GTCATTCCCGCGAAAGCGGGAATC.

43. The composition of any one of items 35 to 42, wherein the single-stranded nucleic acid molecule comprises the following nucleotide sequence: GTCANNCCNGNNNANNONGGNNNC.

44. The composition of item 43, wherein the single-stranded nucleic acid molecule comprises the following nucleotide sequence:

GTCAYBCCMGYRHAVRCKGGVRNC.

45. The composition of item 43 or 44, wherein the single-stranded nucleic acid molecule comprises any one of the nucleotide sequences depicted in FIG. 8 and FIG. 9C.

46. The composition of any one of items 35 to 44, further comprising the single-stranded nucleic acid molecule defined in any one of items 41 to 45.

47. A method for cleaving a single-stranded nucleic acid molecule, the method comprising contacting the single-stranded nucleic acid molecule with the isolated endonuclease of any one of items 1 to 34 or the composition of any one of items 35 to 45 under conditions suitable for cleavage of the single-stranded nucleic acid molecule by the isolated endonuclease, wherein the single-stranded nucleic acid molecule comprises a recognition sequence for the isolated endonuclease.

48. The method of item 47, wherein said conditions comprises a temperature of about 20 to about 55° C.

49. The method of item 48, wherein said conditions comprises a temperature of about 35 to about 40° C.

50. The method of item 49, wherein said conditions comprises a temperature of about 37° C.

51. The method of any one of items 47 to 50, wherein said conditions comprises the presence of a metal.

52. The method of item 51, wherein said metal is magnesium, manganese or nickel.

53. The method of item 52, wherein the metal is magnesium.

54. The method of item 53, wherein the magnesium is in the for magnesium chloride (MgCl₂).

55. The method of any one of item 51 to 54, wherein said metal is at a concentration of at least 5 mM.

56. The method of any one of items 51 to 54, wherein said metal is at a concentration of at least 10 mM.

57. The method of any one of items 47 to 56, wherein said contacting is for a period of at least 2 minutes.

58. The method of any one of items 47 to 56, wherein said contacting is for a period of at least 15 minutes.

59. The method of any one of items 47 to 58, wherein said conditions comprises a pH of about 6 to 8.

60. The method of any one of items 47 to 58, wherein the [concentration of endonuclease]/[single-stranded nucleic acid molecule] ratio is at least 0.00001.

61. The method of item 60, wherein the [concentration of endonuclease]/[single-stranded nucleic acid molecule] ratio is at least 0.01.

62. The method of item 61, wherein the [concentration of endonuclease]/[single-stranded nucleic acid molecule] ratio is at least 0.5.

63. A method for rendering a single-stranded nucleic acid susceptible to cleavage by the endonuclease defined in any one of items 1 to 34, the method comprising incorporating a nucleotide sequence comprising a recognition sequence for the isolated endonuclease into the single-stranded nucleic acid.

64. The method of item 63, wherein the nucleotide sequence comprises one of the sequence defined in any one of items 42 to 45

65. The method of item 64, wherein the method comprises adding a nucleic acid fragment comprising the nucleotide sequence defined in any one of items 42 to 45 at the 5′-end, 3′-end or within the single-stranded nucleic acid.

66. The method of item 64, wherein the method comprises introducing one or more mutations within the sequence of the single-stranded nucleic acid to obtain the nucleotide sequence defined in any one of items 42 to 45.

67. A cell comprising the endonuclease defined in any one of items 1 to 34, wherein the endonuclease is heterologous to the cell.

68. A method for expressing the endonuclease defined in any one of items 1 to 34 in a cell, the method comprising introducing a nucleic acid encoding the endonuclease into the cell.

69. The cell of item 67 or the method of item 68, wherein the cell is a prokaryotic or eukaryotic cell.

70. The cell or method of any one of items 67 to 69, wherein the cell comprises a single-stranded nucleic acid that is cleaved by the endonuclease.

71. The cell or method of any one of items 68 to 70, wherein the nucleic acid encoding the endonuclease is present in a vector.

72. A kit comprising the endonuclease defined in any one of items 1 to 34 or the composition of any one of items 35 to 45, and instructions for cleaving single-stranded nucleic acid molecules using the endonuclease.

73. The kit of item 72, wherein said instructions comprise the method of any one of items 47 to 62.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A depicts purification of SsnA from N. meningitidis, N. elongata, R. felis and L. pneumophila along with three mutant proteins from N. meningitidis. Proteins were fused to a N-terminal 6×His-tag or a N-terminal GST tag by cloning into pET15-MHL and pGEX vectors respectively, then purified to near purity by affinity chromatography.

FIG. 1B depicts the amino acid sequence of SsnA from N. meningitidis (SEQ ID NO:2) along with its sequence features. Individually mutated residues are highlighted.

FIG. 2A shows that SsnA is a metal-dependent endonuclease. Results of an endonuclease assay of 1 μM SsnA on a 100 nt specific ssDNA. Rows #1-3 were performed using commercial NEBuffer2.1 (containing 10 mM MgCl₂), supplemented with 10 mM EDTA where indicated (#3). Rows #4-5 were performed using a homemade identical buffer lacking MgCl₂, which was supplemented with 10 mM MgCl₂ where indicated (#5).

FIG. 2B shows that SsnA only interacts with single-stranded DNA containing a specific sequence (NTS). Gel-shift (top, EMSA) and endonuclease (bottom) assays of 0.5 μM SsnA with 0.8 UM of DNA. Magnesium was omitted from the EMSA reaction mix to allow DNA binding by SsnA without nuclease activity. ssDNArev is the reverse complement of ssDNA. dsDNA is the double-stranded annealed product from ssDNA with ssDNArev. The arrow represents the recognition pattern (NTS repeat) of SsnA. Unless otherwise indicated (last well), assayed proteins were his-tagged.

FIG. 2C shows enzymatic activity of SsnA on truncated ssDNA and RNA. Sequences of 75 nt, 37 nt and 28 nt were assayed, all of them containing a complete NTS sequence. The 37 nt RNA sequence is the transcribed equivalent to the 37 nt DNA. The 75 nt ssDNA (T→U) sequence corresponds to the 75 nt ssDNA with uracils instead of thymines (but with desoxyribose sugars).

FIG. 3 depicts the binding and cleavage of different ssDNA by SsnA. 100 nt oligonucleotides containing the full-length NTS repeat with different flanking sequences were assayed. Each sequence was taken from the N. meningitidis genome.

FIG. 3B depicts the assayed ssDNA sequences. The NTS repeat region is underlined.

FIG. 4A depicts the cleavage site determination of SsnA on a 75 nt ssDNA containing its target sequence. 5′-labelled oligonucleotides of 18 to 25 nt were run on a gel next to the results of an endonuclease assay with SsnA.

FIG. 4B depicts the sequence requirements for binding of cutting of SsnA on ssDNA. Gel-Shift (binding activity) and endonuclease (cutting activity) assays were performed on 75 nt ssDNA containing the target sequence (NTS), with single-nucleotide mutations throughout its length. The relative activities were measured for each individually mutated sequence and illustrated using a heat-map. Cleavage activity was normalized to the binding activity since binding is a prerequisite for cutting. Arrows denote the palindrome within the repeated sequence, which forms the stem of a stem-loop structure. Scissors depict the cutting site identified in FIG. 5.

FIG. 5 depicts the absence of binding activity of SsnA on branched DNA. Gel-shift assays (EMSA) of different branched DNA structures in presence of SsnA. H: Holliday junction, D: D-loop, F: fork, Y: pseudo-Y.

FIGS. 6A-D show the nuclease activity of SsnA. FIG. 6A: Metal requirements for the nuclease activity of SsnA. 10 mM of each metal was used as the sole metal in the reaction mix. GST-tagged SsnA was assayed with nickel to ensure that the activity seen with His-SsnA was not due to nickel interacting with the purification tag.

FIG. 6B: Magnesium and manganese requirements for the nuclease activity of SsnA.

FIG. 6C: Temperature effect on SsnA's nuclease activity. For all of the above experiments, 0.8 μM of 100 nt ssDNA containing the recognition sequence was used with 1 μM of His-SsnA WT unless otherwise indicated. Boxed images show the specific cleavage products that were obtained and quantified.

FIG. 6D: depicts the cleavage kinetics of SsnA with the indicated concentrations of ssDNA.

FIG. 6E: Dose-response nuclease assay of SsnA depicting its sensitivity.

FIGS. 7A-C show the maximum likelihood mid-rooted phylogeny of SsnA and the Ssn protein family (GIY-YIG small proteins). Proteins NMV_0044 (SsnA) and NMV_0402 (SsnB, circle) from N. meningitidis 8013 2C4.3 was blasted against all bacteria with a threshold of 50% identity. Results were screened and curated to only keep proteins of 80-120 amino acids, corresponding to single GIY-YIG-domain proteins. FIG. 7B shows the portion of the tree corresponding to SsnA homologs from the Neisseriaceae family. FIG. 7C shows the portion of the tree corresponding to SsnB homologs from the Neisseriaceae family.

FIG. 8 shows the alignment of the putative recognition sequences of SsnA homologs, all neighbouring the SsnA gene in the corresponding species. The recognition sequence of SsnANm was blasted against the genomic regions (10 kbp) encompassing homologous genes from other species, then aligned with AlignX from the Vector NTI software.

FIG. 9A shows an alignment of the amino acid sequences of SsnA nucleases from N. meningitidis 8013 2C4.3 (SsnA(NMV0044), SEQ ID NO:2), Neisseria elongata subsp. glycolytica (SsnA(EFE49965.1), SEQ ID NO:3), Legionella pneumophila subsp. pneumophila str. Paris SsnA(WP_011213498), SEQ ID NO:4) and Rickettsia felis str. URRWXCal2 (SsnA(WP_011271370), SEQ ID NO:5), performed using Clustal Omega 1.2.4. Conserved amino acids are indicated by asterisks (*), the residues with strong similarity (PAM250 MATRIX score between amino acids of greater than 0.5) indicated by colons (:), and the residues with weak similarity (PAM250 MATRIX score between amino acids of 0.5 or less) indicated by dots (.).

FIG. 9B depicts the percent identity matrix of SsnA homologs aligned above.

FIG. 9C: Sequences located near the genes encoding SsnA homologs and having similarities with the recognition sequence from N. meningitidis were identified and aligned using Clustal Omega. Conserved nucleotides are highlighted, with darker tones highlighting the most conserved positions.

FIG. 10A-C show the nuclease activity of SsnA homologs from other species. Sequences located near the genes encoding these SsnA and having similarities with the recognition sequence from N. meningitidis were identified and synthesized with a 5′ fluorescent tag, then tested with their respective SsnA using nuclease assays as previously described. FIG. 10A depicts the nuclease activity of SsnA from N. elongata (EFE49965.1), FIG. 10B depicts the nuclease activity of SsnA from R. felis (WP_011271370) and FIG. 10C depicts the nuclease activity of SsnA from L. pneumophila (WP_011213498).

FIGS. 11A-11JJJJ depicts the amino acid sequences of various putative Ssn having at least 50% sequence identity with Protein NMV_0044 (SsnA) from N. meningitidis 8013 2C4.3 (SEQ ID NO: 2).

FIGS. 12A-B show quantitative transformation assays performed on wild-type (WT), SsnA knock-out (KO) and SsnA complemented (Compl) strains of N. meningitidis. FIG. 13A: transformation of a plasmid that does not harbour the nuclease's recognition sequence. FIG. 13B: transformation of a plasmid harbouring SsnA's recognition sequence.

FIGS. 13A-B show an alignment of the amino acid sequences of 79 endonucleases comprising a GIY-YIG domain belonging to the GIY-YIG_unchar_3 Conserved Protein Domain Family (CD10448) (Lu S et al. (2020). “CDD/SPARCLE: the conserved domain database in 2020.”, Nucleic Acids Res. 48 (D1): D265-D268)

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The present inventors have identified a family of endonucleases that preferentially bind to and cleave single-stranded DNA. These endonucleases recognize and cleave specific nucleotide sequences in single-stranded nucleic acids (single-stranded DNA). These endonucleases may be useful, for example, in various genetic engineering and molecular biology applications. These endonucleases are short proteins (typically less than 150 amino acids, and preferably less than 140 or 130 amino acids) and comprises a conserved GIY-YIG domain. The GIY-YIG domain comprises two short semi-conserved motifs “GIY” and “YIG” in the N-terminal part, followed by an Arg residue in the center and a Glu residue in the C-terminal part. The GIY-YIG domain has an α/β-sandwich architecture with a central three-stranded antiparallel β-sheet flanked by three-helices. The three-stranded antiparallel β-sheet contains the GIY-YIG sequence elements.

The present disclosure provides an isolated endonuclease specific for single-stranded nucleic acid molecules (e.g., binds to and cleaves a single-stranded nucleic acid such as single-stranded DNA), the isolated endonuclease having a length of 150 amino acids or less and comprising a GIY-YIG domain.

The present disclosure also provides a cell comprising an endonuclease specific for single-stranded nucleic acid molecules as described herein. In an embodiment, the endonuclease is heterogenous to the cell. “Heterogenous” as used herein means that the endonuclease is the product of a gene that is not naturally present in the cell. For example, if the endonuclease is the endonuclease of SEQ ID NO:2, the cell is not a Neisseria elongata subsp. glycolytica cell. The cell may be a cell from another bacterial species or subspecies or an eucaryotic cell (mammalian cell, human cell, yeast cell, etc.), for example.

In an embodiment, the endonuclease of the present disclosure has a length of 80 to 130 amino acids. In another embodiment, the endonuclease of the present disclosure has a length of 85 to 120 amino acids. In an embodiment, the endonuclease of the present disclosure does not comprise any additional domain, it only consists of a single GIY-YIG domain.

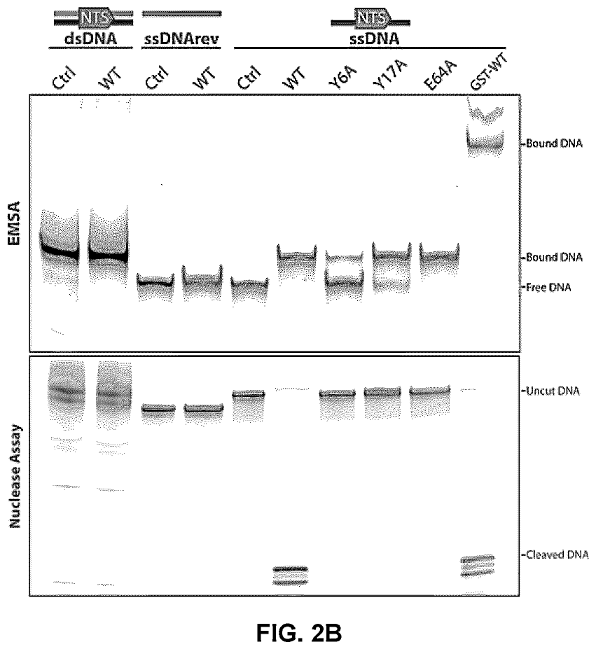

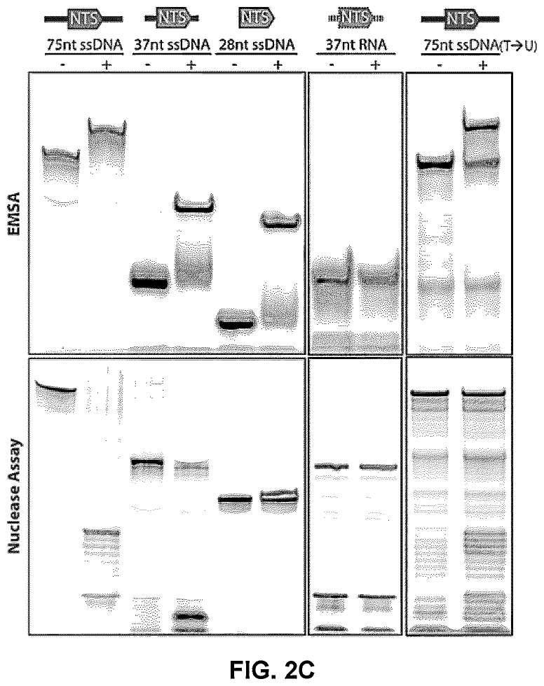

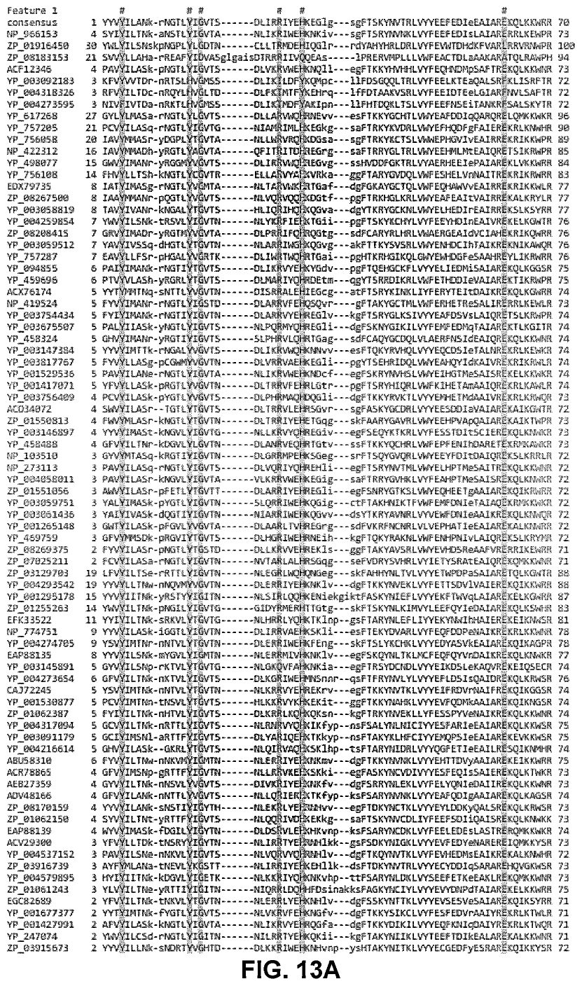

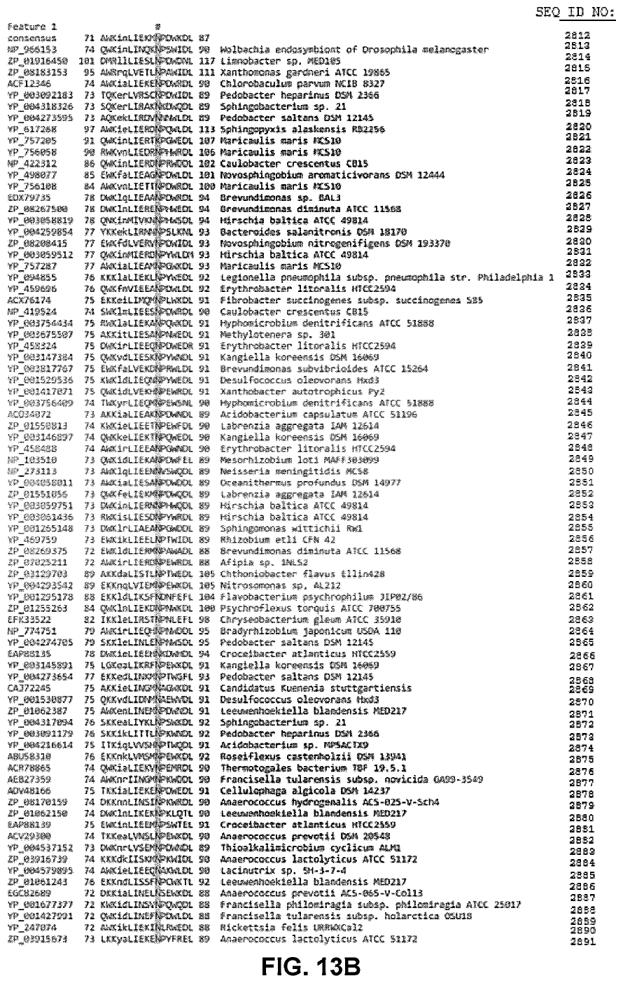

FIGS. 13A-B depicts a sequence alignment of 79 representative GIY-YIG domains from short endonucleases according to the present disclosure. These 79 endonucleases belong to the GIY-YIG_unchar_3 Conserved Protein Domain Family (CD10448) (Lu S et al. (2020). “CDD/SPARCLE: the conserved domain database in 2020.”, Nucleic Acids Res. 48 (D1): D265-D268). FIG. 2A also depicts the sequences corresponding to the GIY motif, the YIG motif and the conserved Glu residue (putative metal binding site) in SsnA from N. meningitidis (SEQ ID NO:2).

In an embodiment, the endonuclease of the present disclosure comprises a GIY-YIG domain of the formula I:

X1-X2-X3-B1-X4-X5-X6-B2-X7-B3-X8 (I)
wherein
X1 is any amino acid, preferably G, Y, W, V, A, F, I, C, H, R, T, S, more preferably Y
X2 is any amino acid, preferably V, I, L, T, A, F, more preferably V
X3 is Y or H, preferably Y
B1 is a sequence of 8 to 12 amino acids, preferably 9 to 11 amino acids, for example 10 amino acids
X4 is Y or H, preferably Y
X5 is any amino acid, preferably I, L, V, T, A, C, or K, more preferably I, T or V, even more preferably I.
X6 is G or D, preferably G;
B2 is a sequence of 6 to 15 amino acids, preferably of 6 to 10 amino acids, more preferably of 6 to 8 amino acids, for example 7 amino acids;
X7 is R or T, preferably R;
B3 is a sequence of 30 to 40 amino acids, preferably of 35 to 40 amino acids, for example 35, 36 or 37 amino acids; and
X8 is E, I or A, preferably E.

In another embodiment, the endonuclease of the present disclosure comprises a GIY-YIG domain of the formula II:

X1-X2-X3-B1-X4-X5-X6-B2-X7-B4-X9-B5-X8 (II)
wherein
X1, X2, X3, B1, X4, X5, X6, B2, X7 and X8 are as defined above;
B4 is a sequence of 1 to 5 amino acids, preferably of 2 to 4 amino acids, more preferably of 3 amino acids;
X9 is H, Q or Y, preferably H; and
B5 is a sequence of 30 to 38 amino acids, preferably of 30 to 35, more preferably of 31 to 33 amino acids, for example 32 amino acids.

In another embodiment, the endonuclease of the present disclosure comprises a GIY-YIG domain of the formula III:

X1-X2-X3-B1-X4-X5-X6-B2-X7-B4-X9-B5-X8-B6-X10 (III)
wherein
X1, X2, X3, B1, X4, X5, X6, B2, X7, B4, X9, B5 and X8 are as defined above;
B6 is a sequence of 15 to 20 amino acids, preferably of 16 to 19 amino acids, more preferably of 18 amino acids; and
X10 is N or K, preferably N.

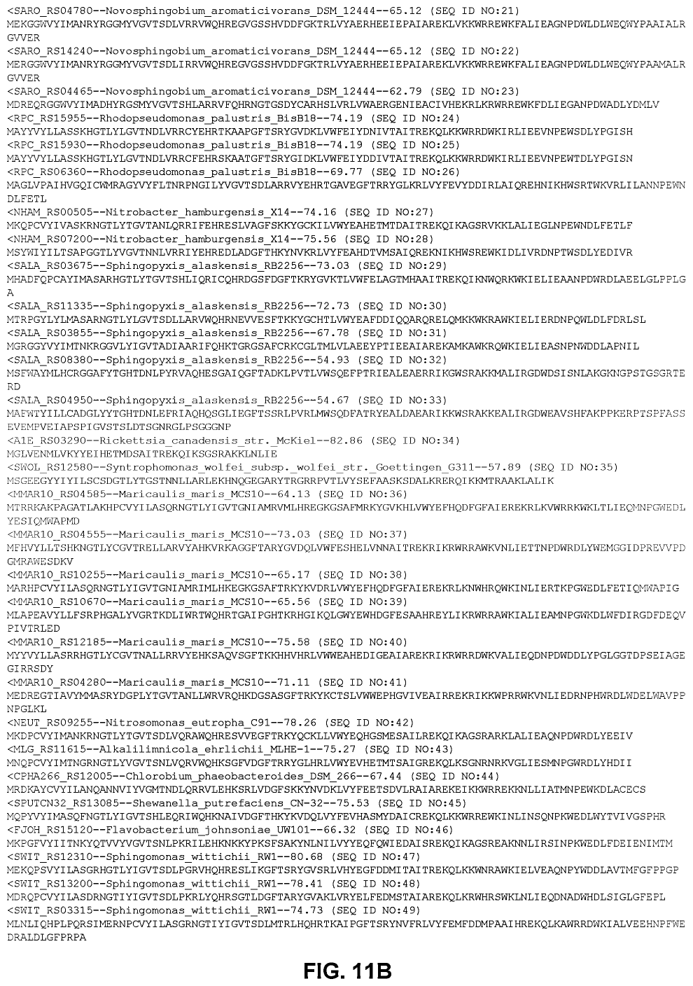

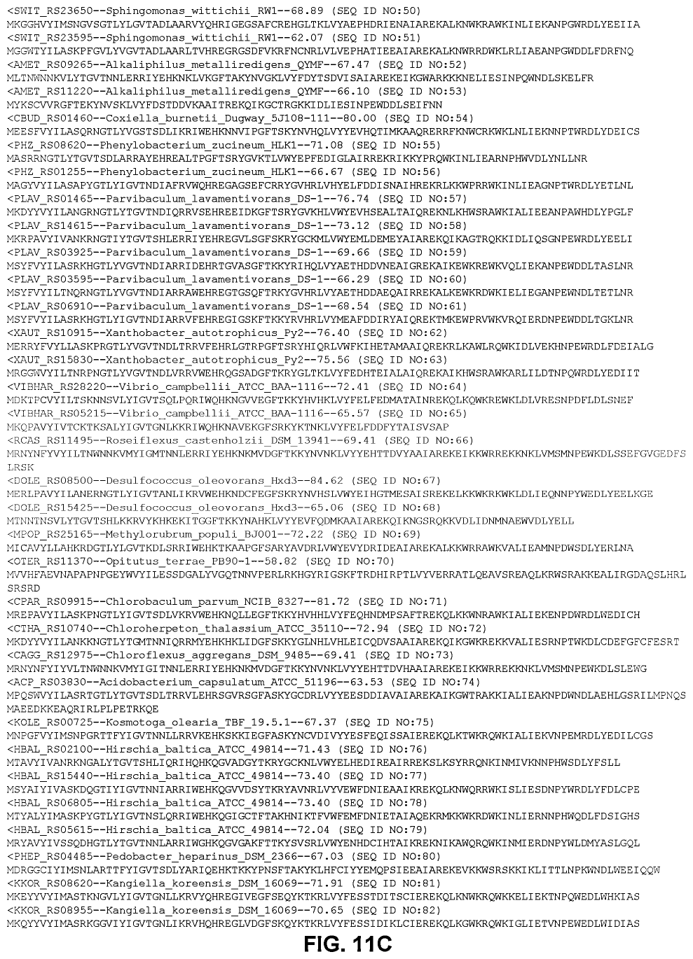

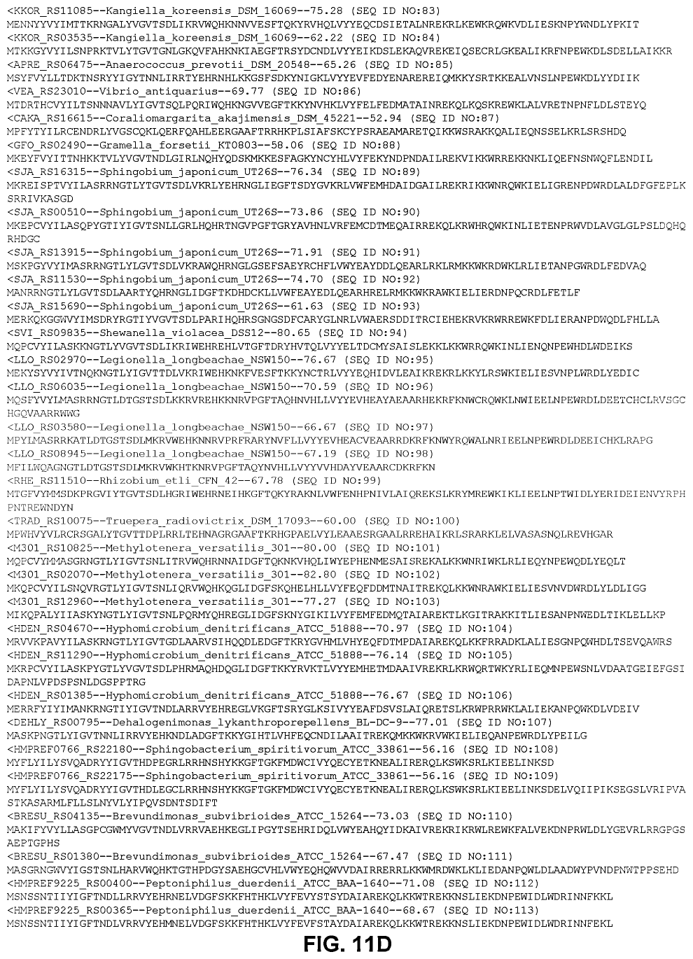

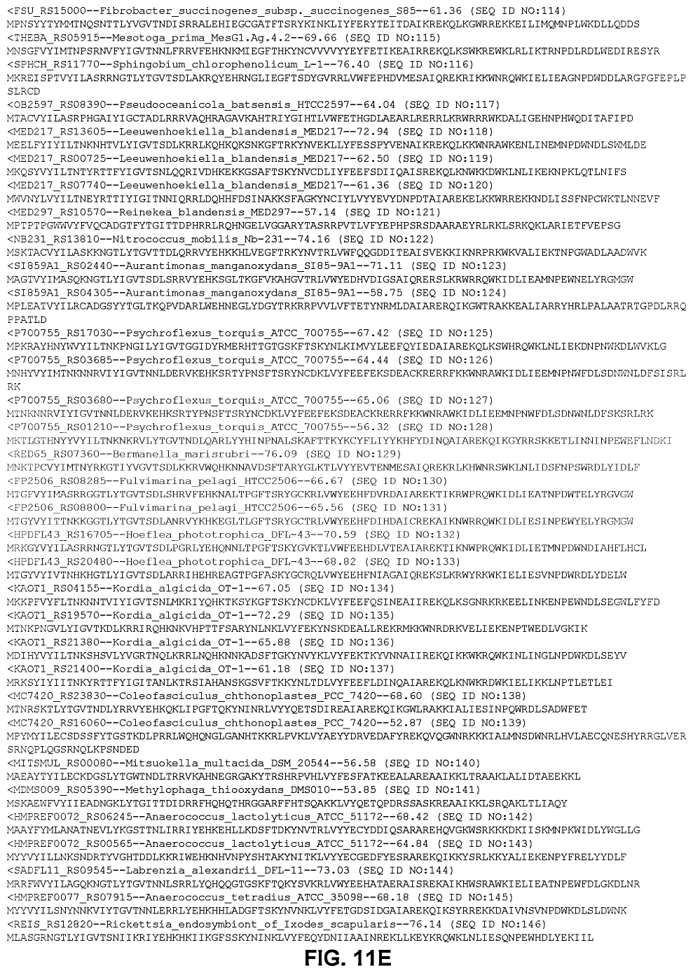

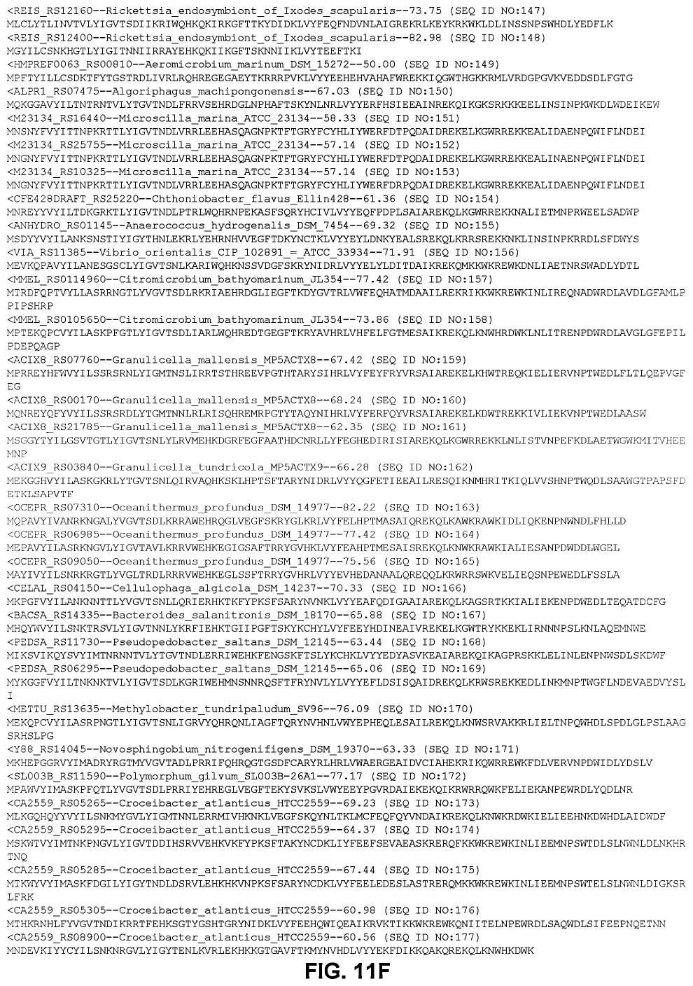

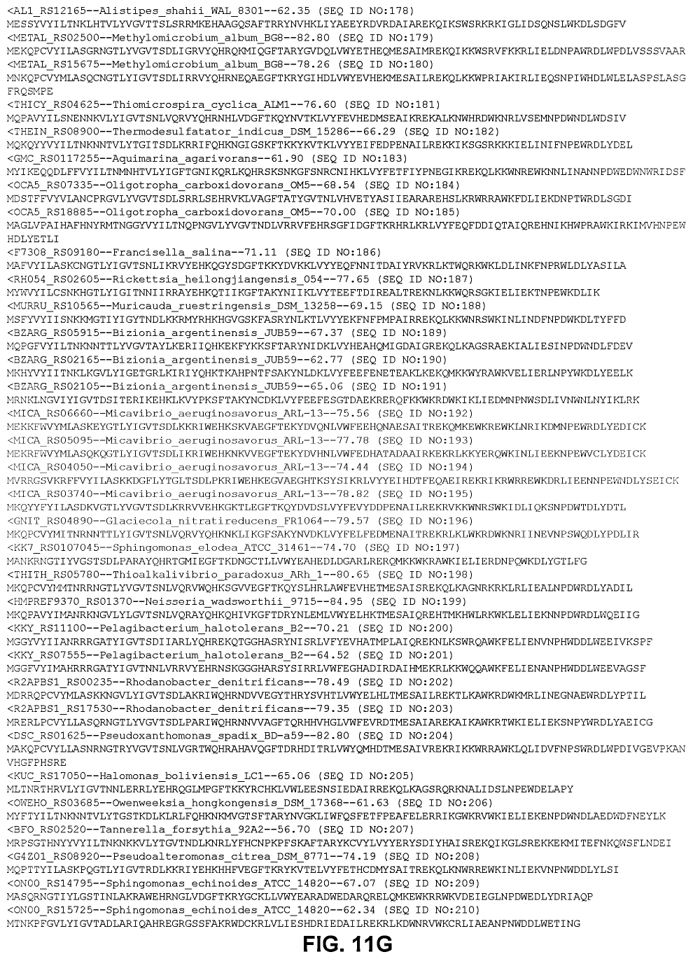

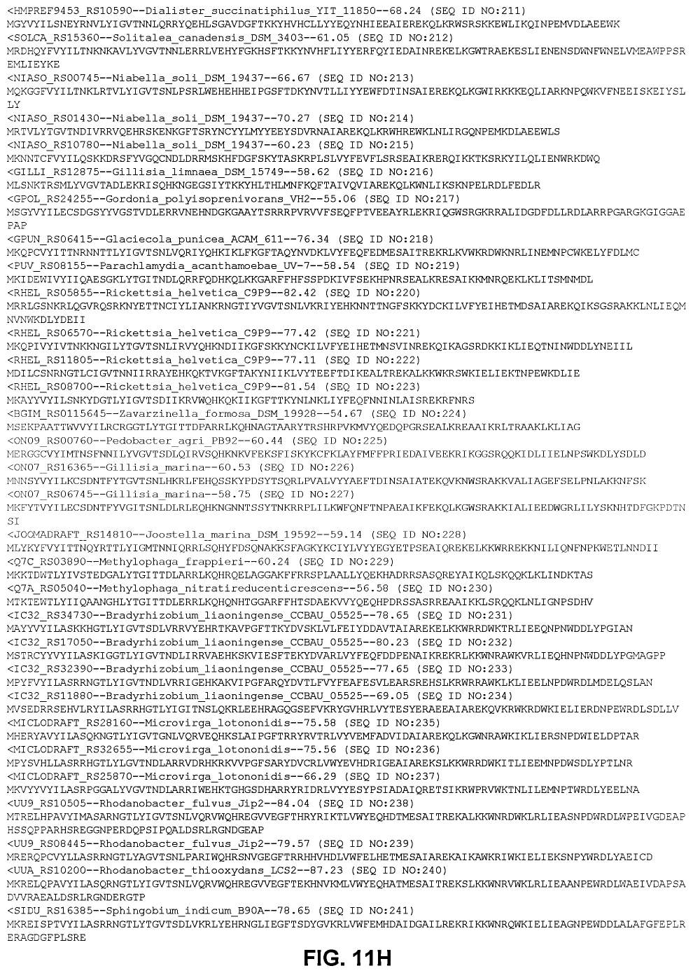

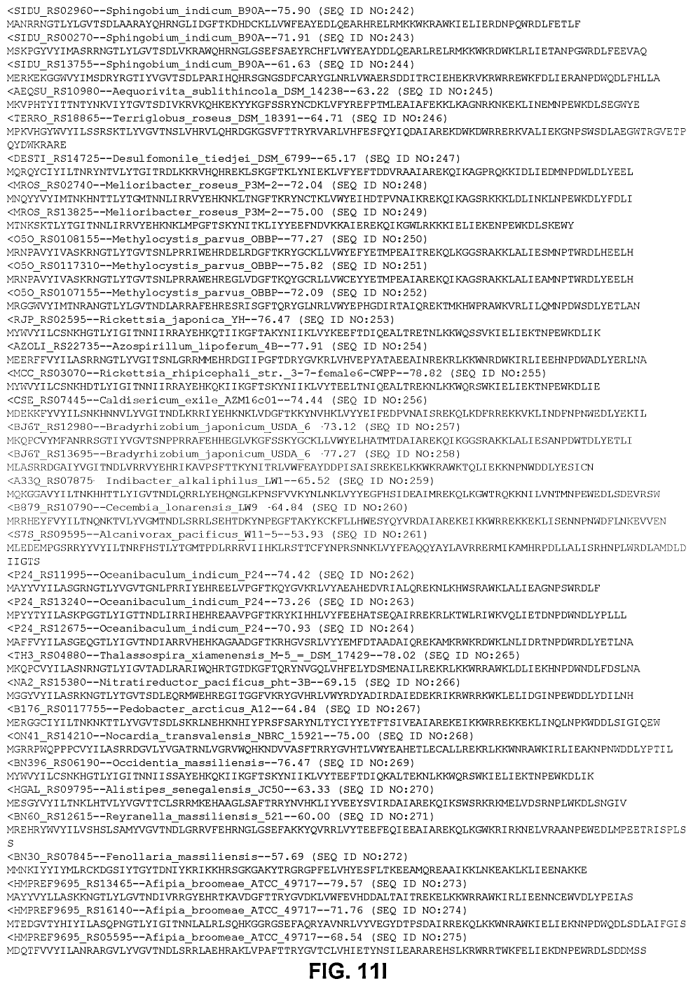

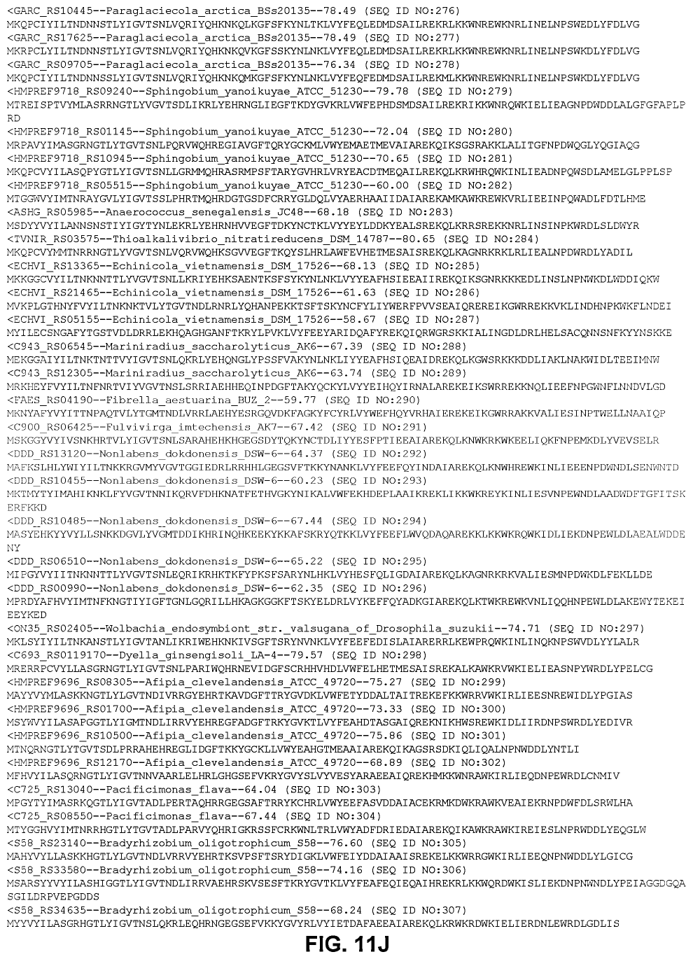

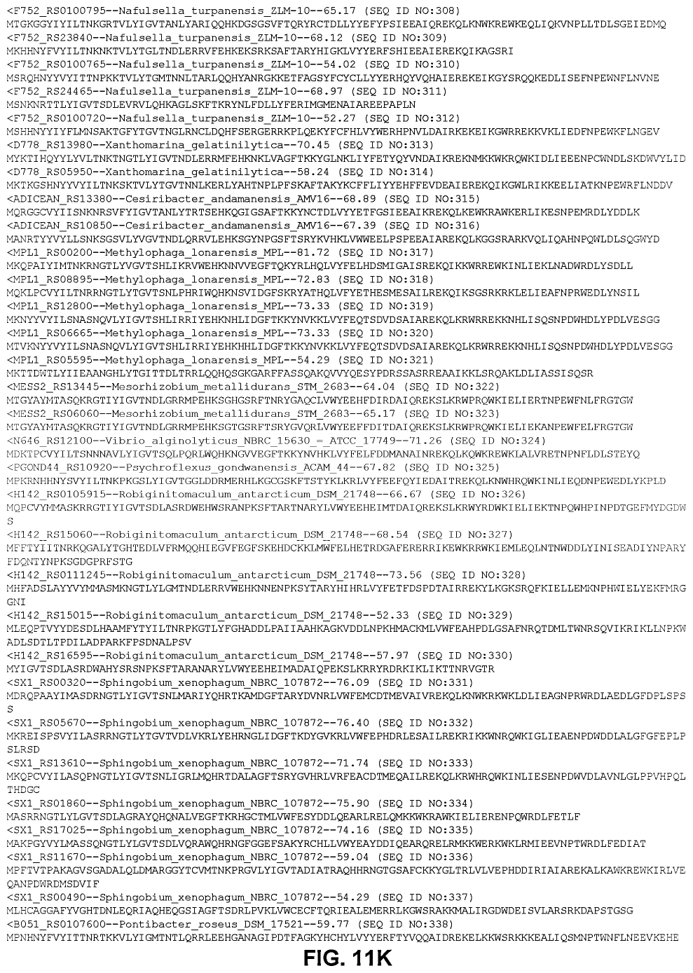

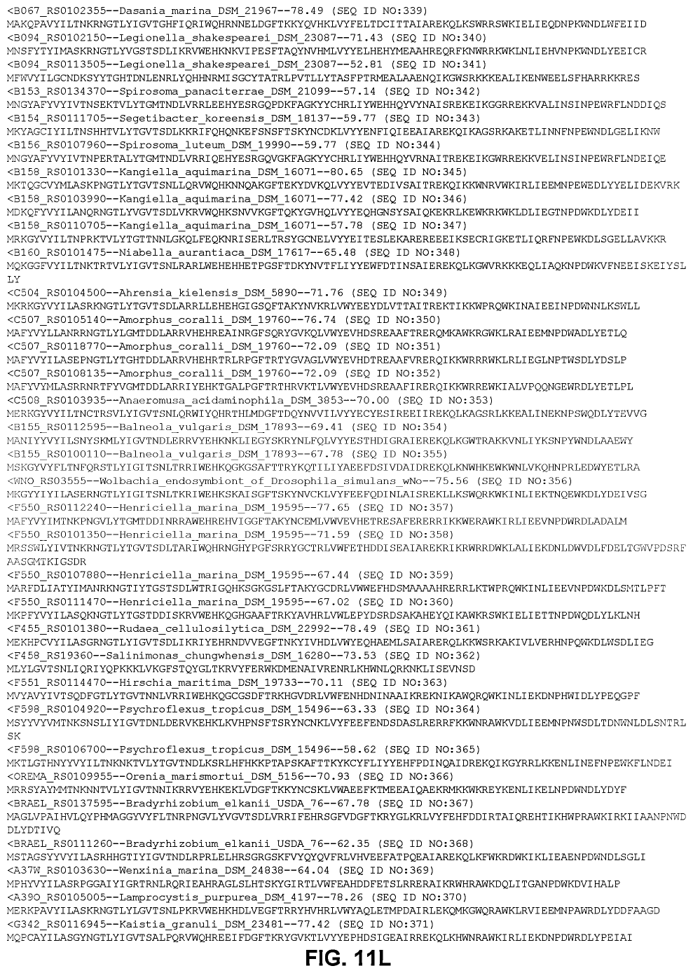

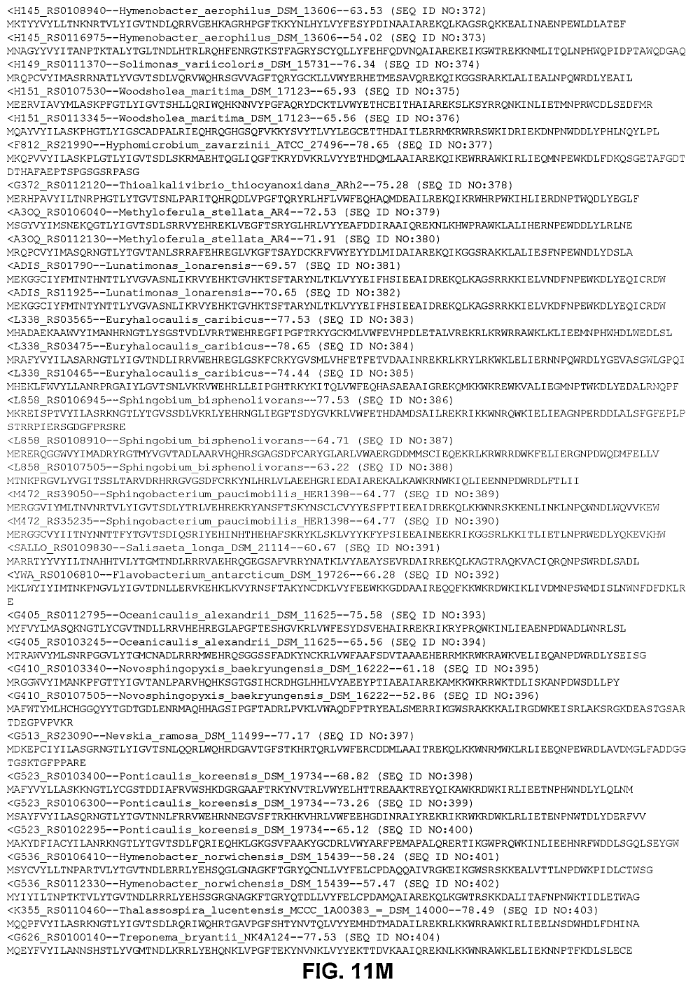

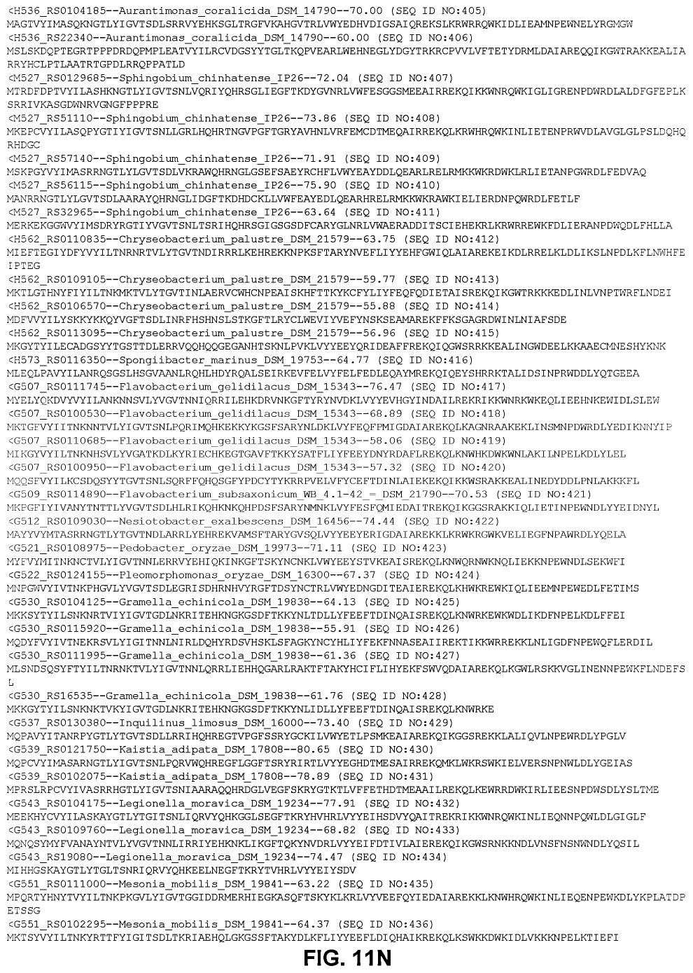

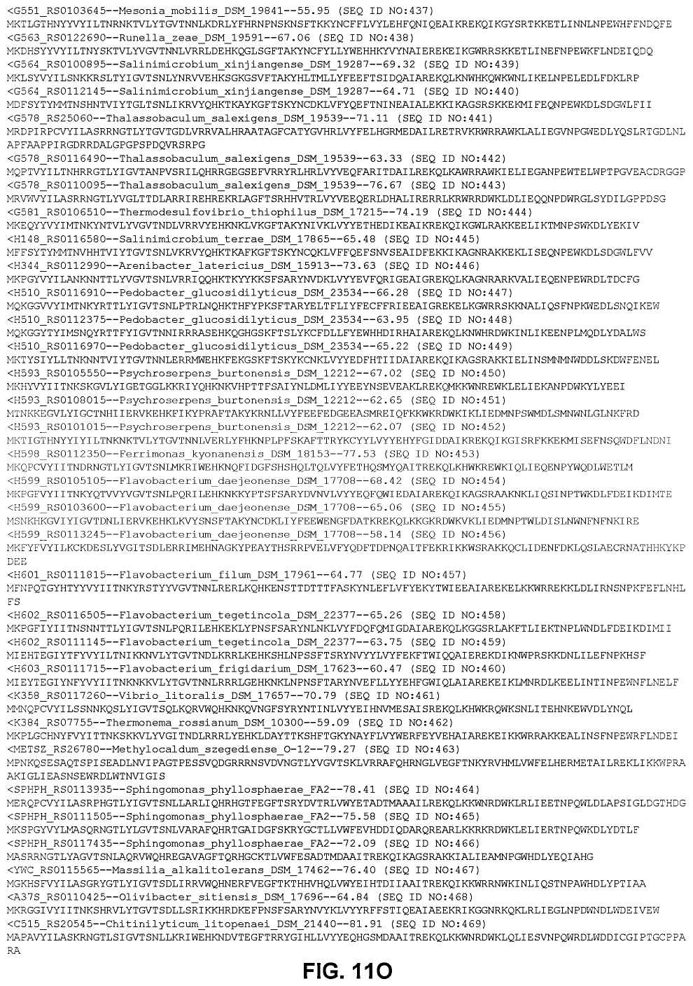

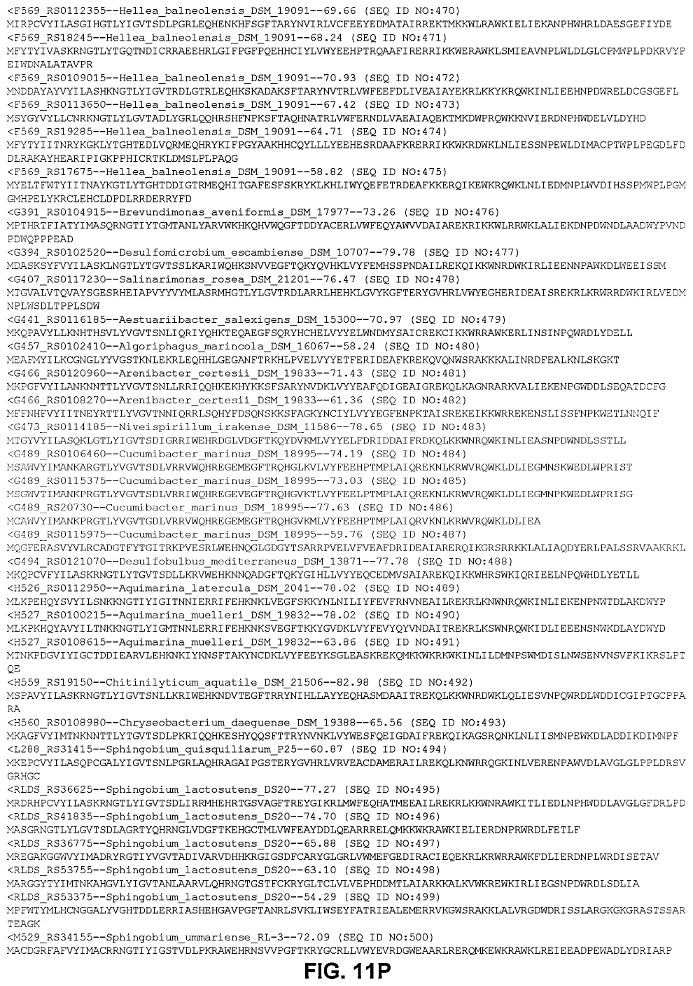

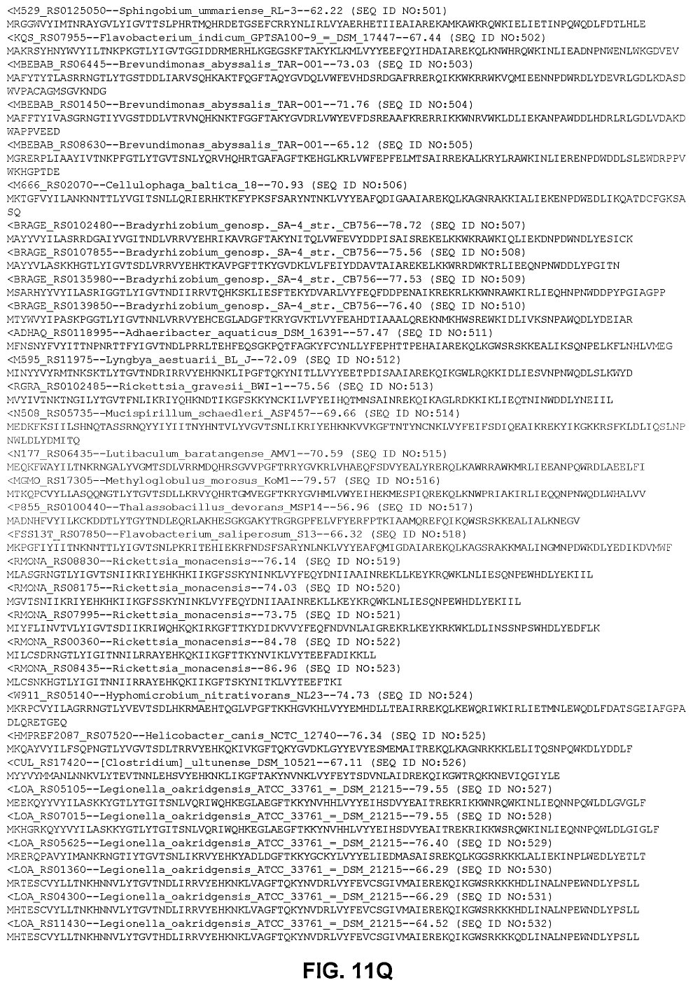

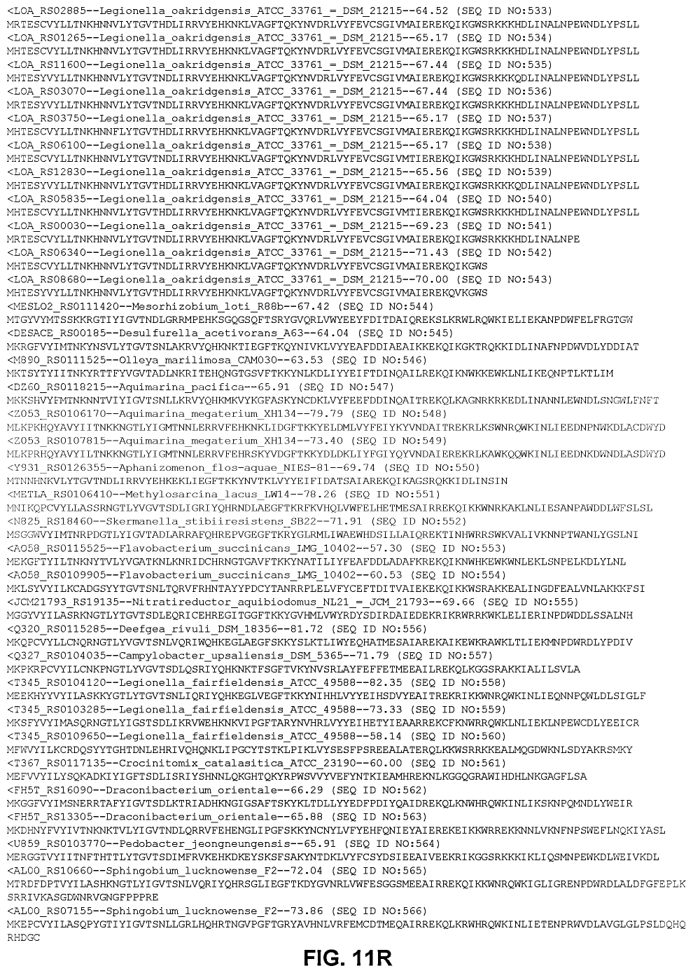

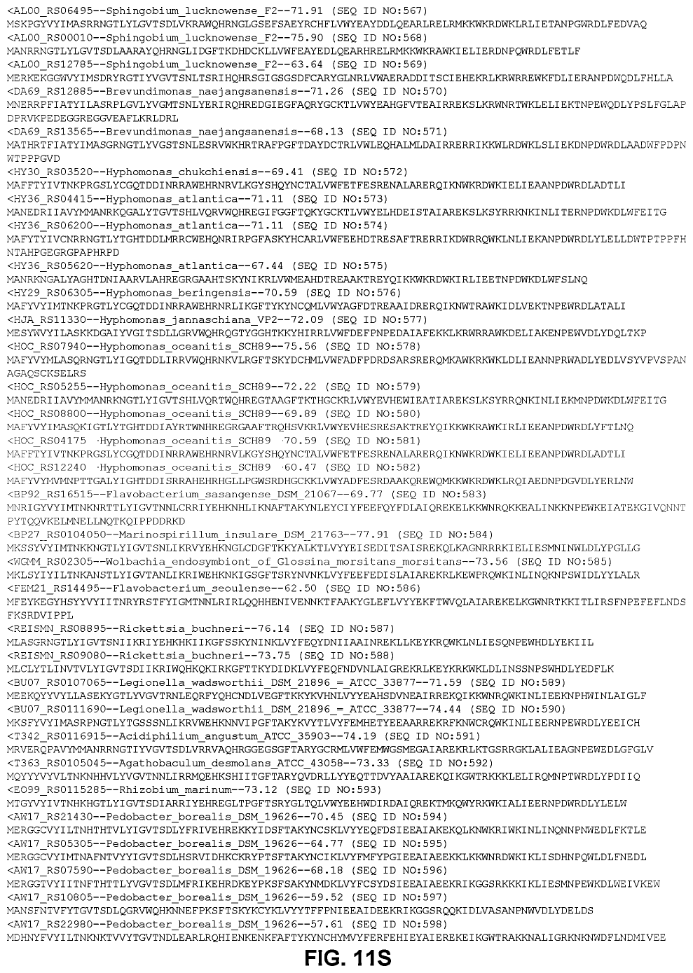

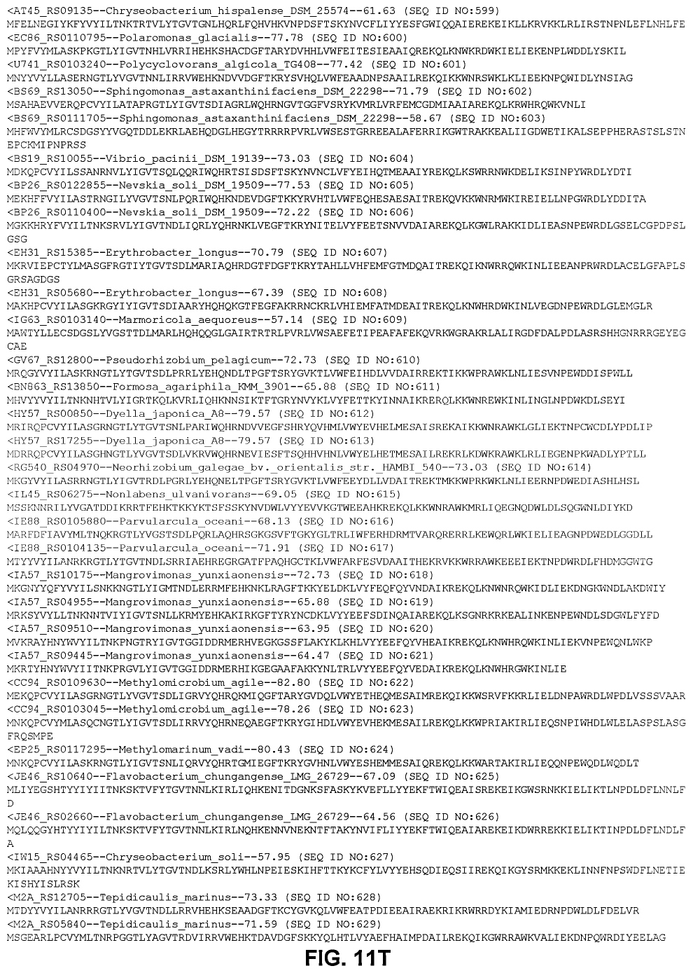

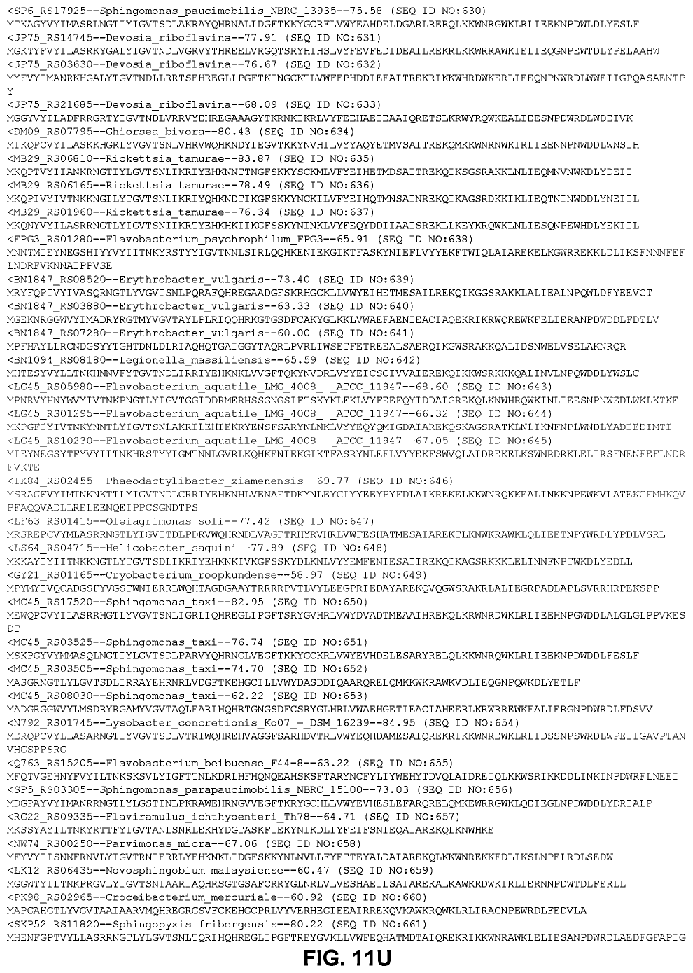

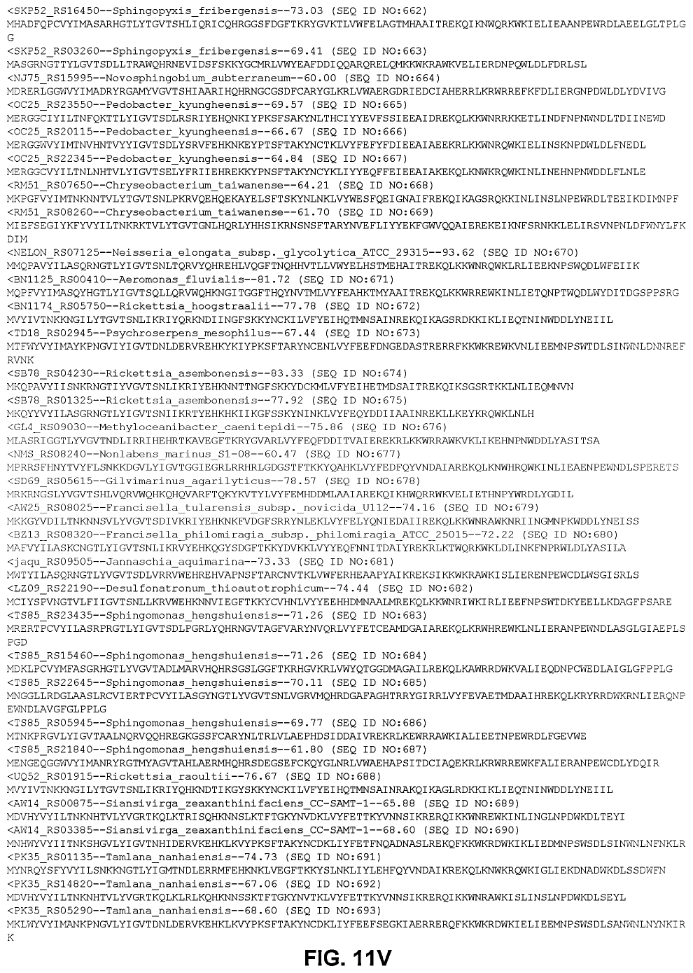

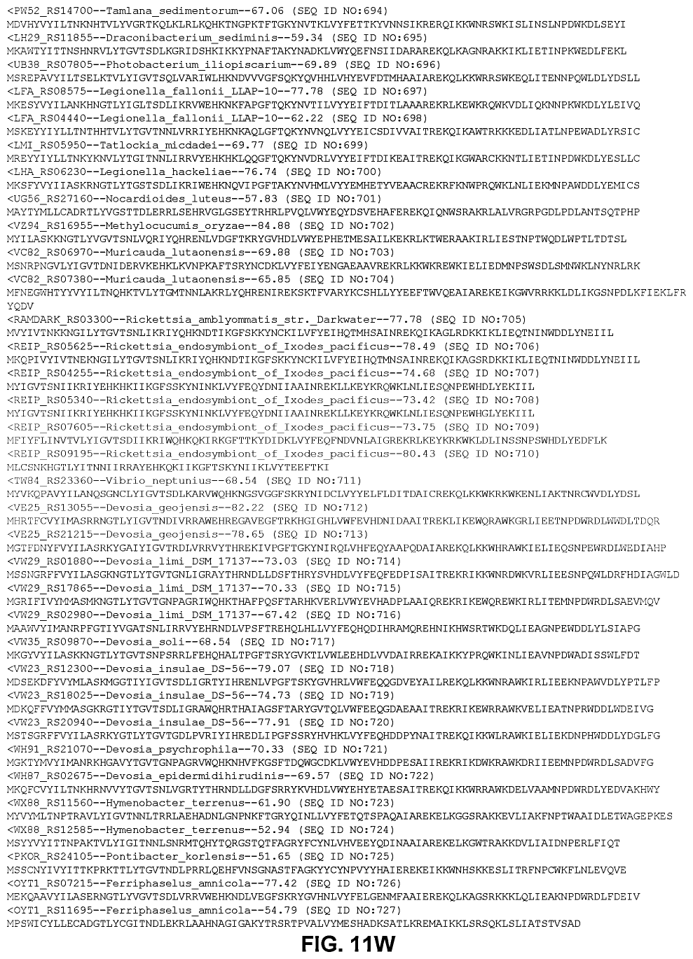

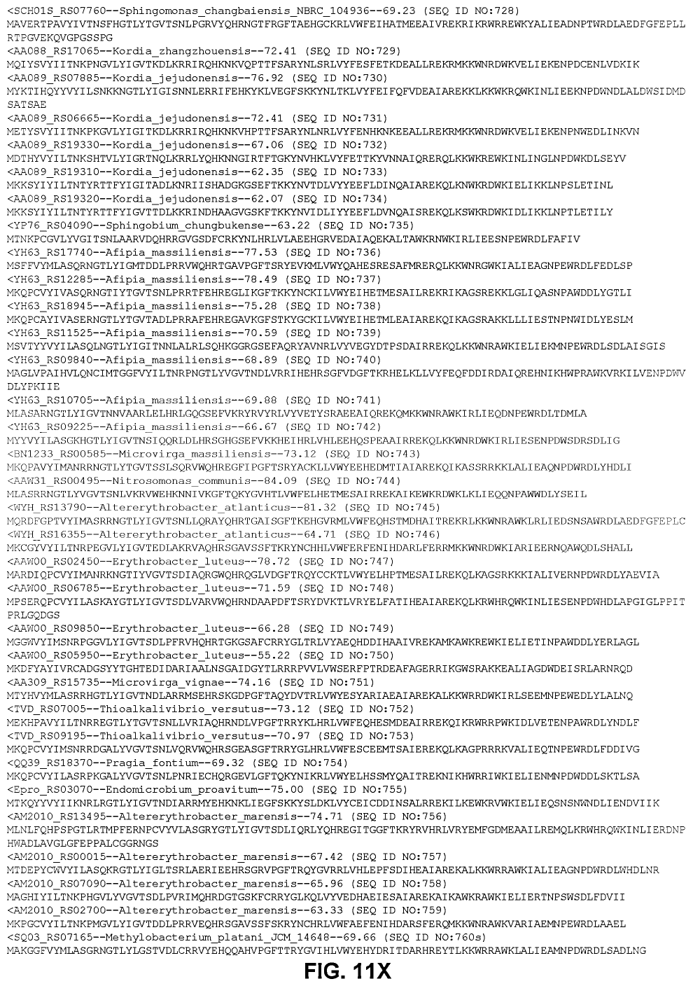

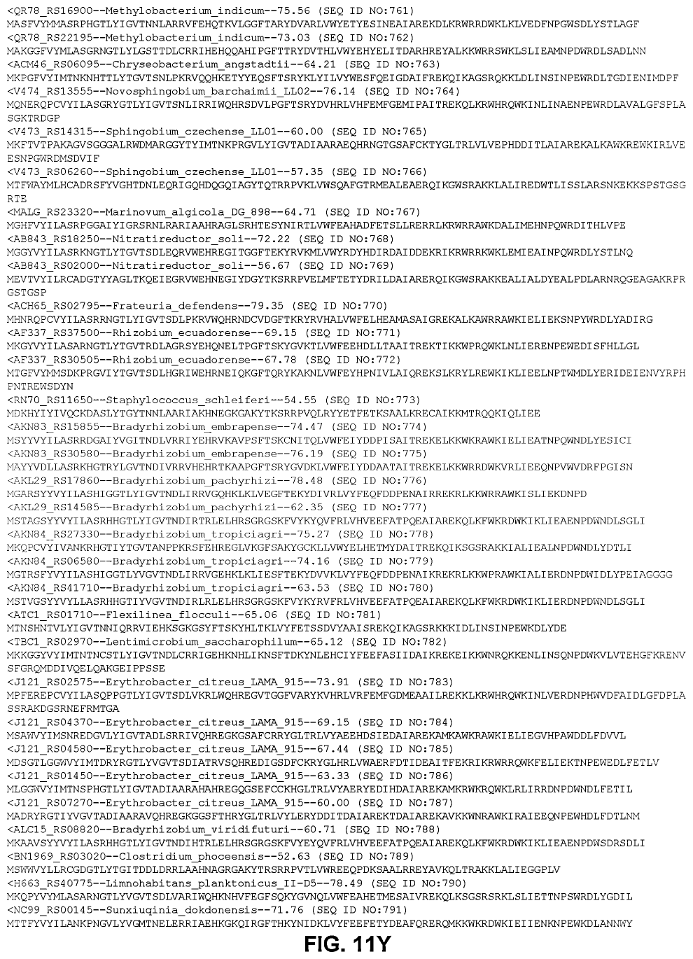

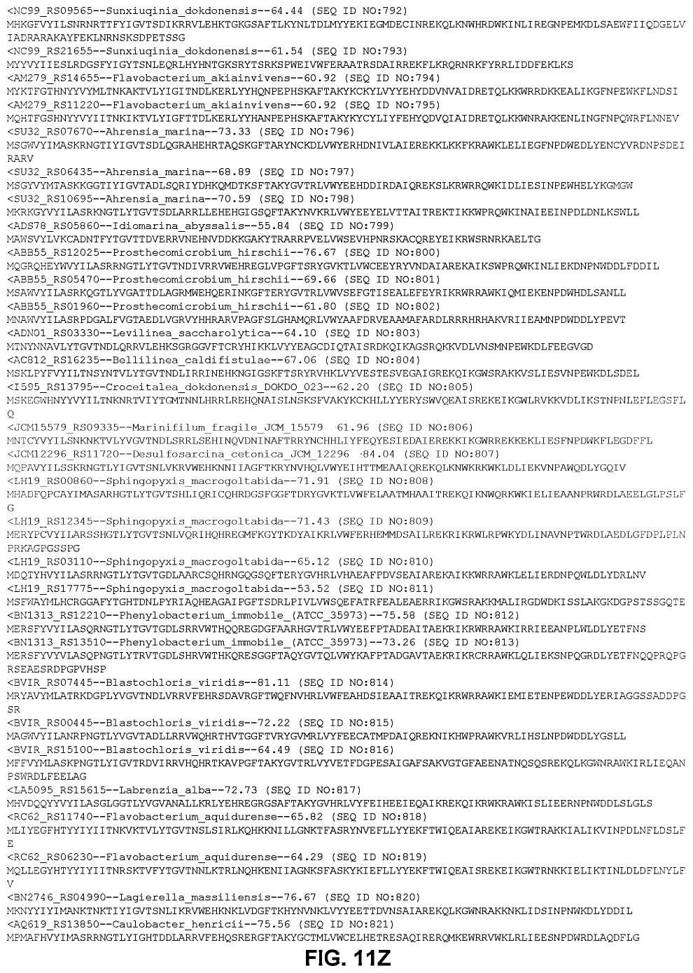

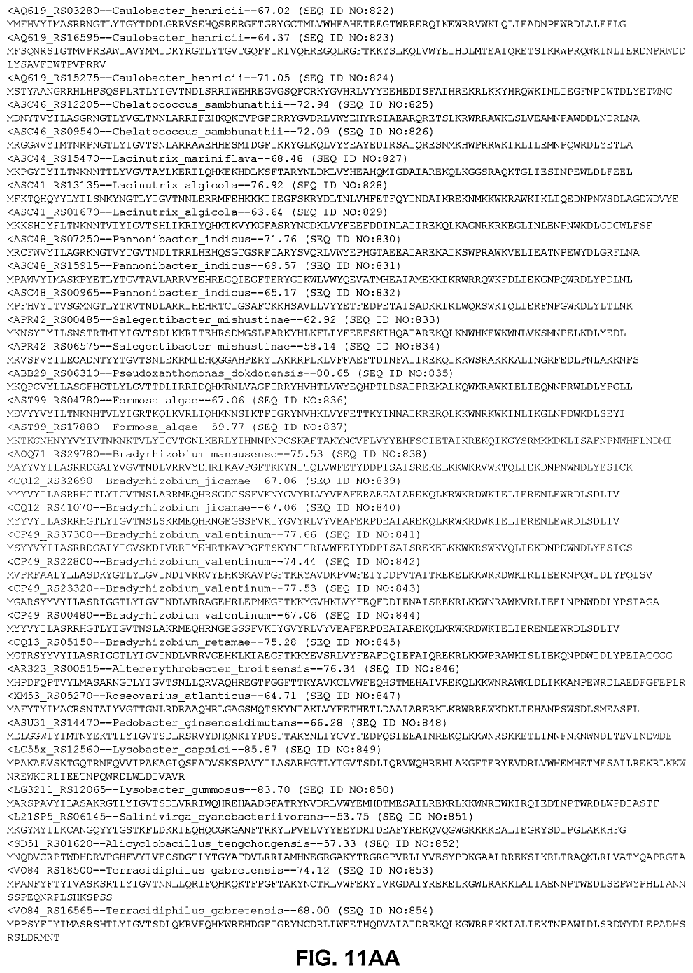

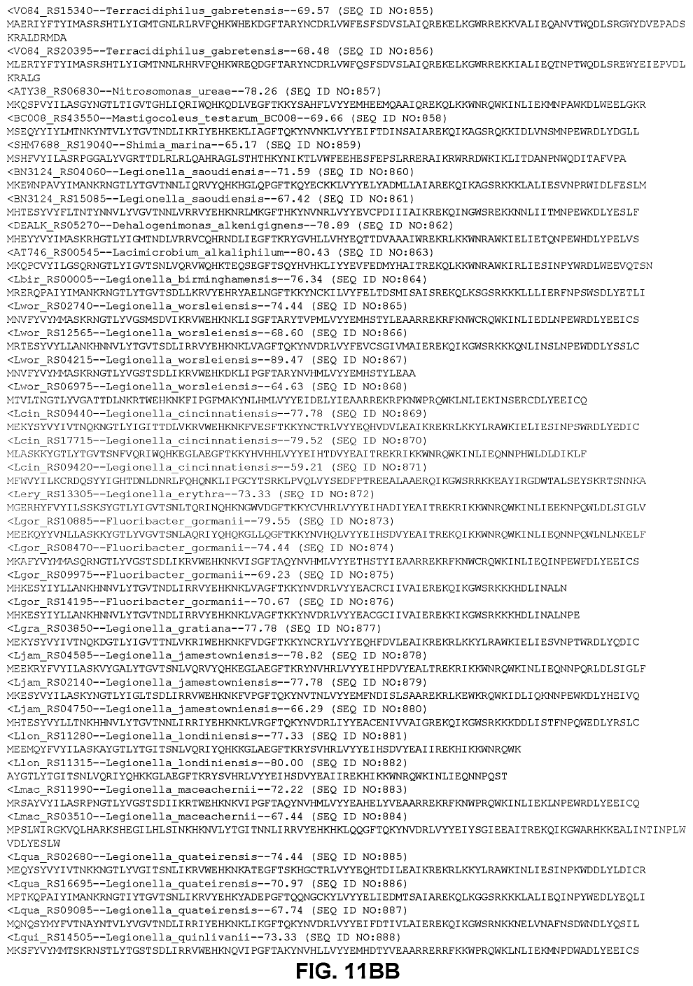

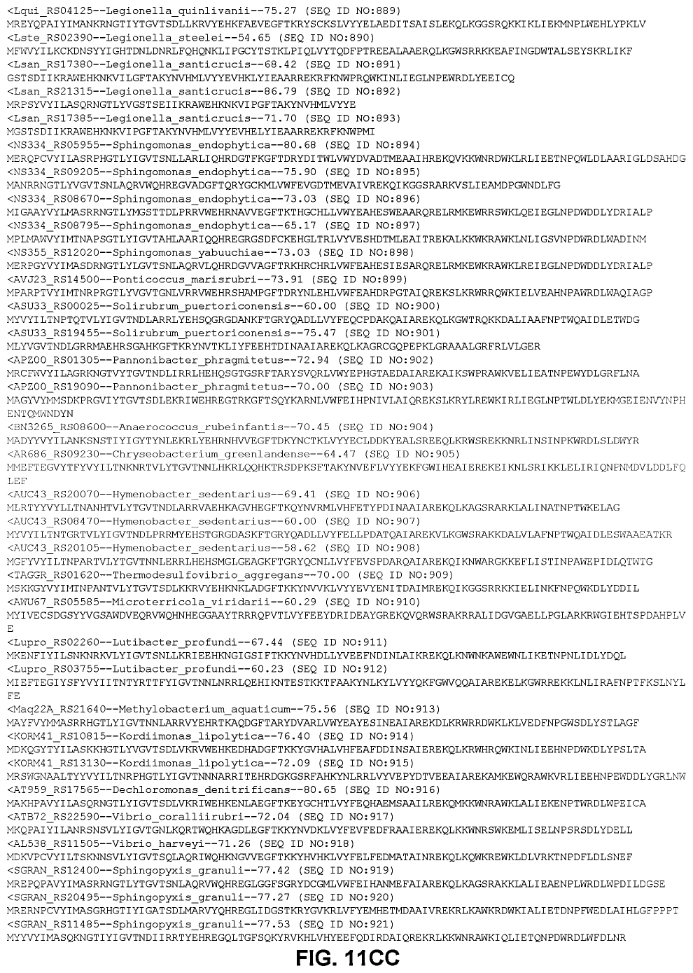

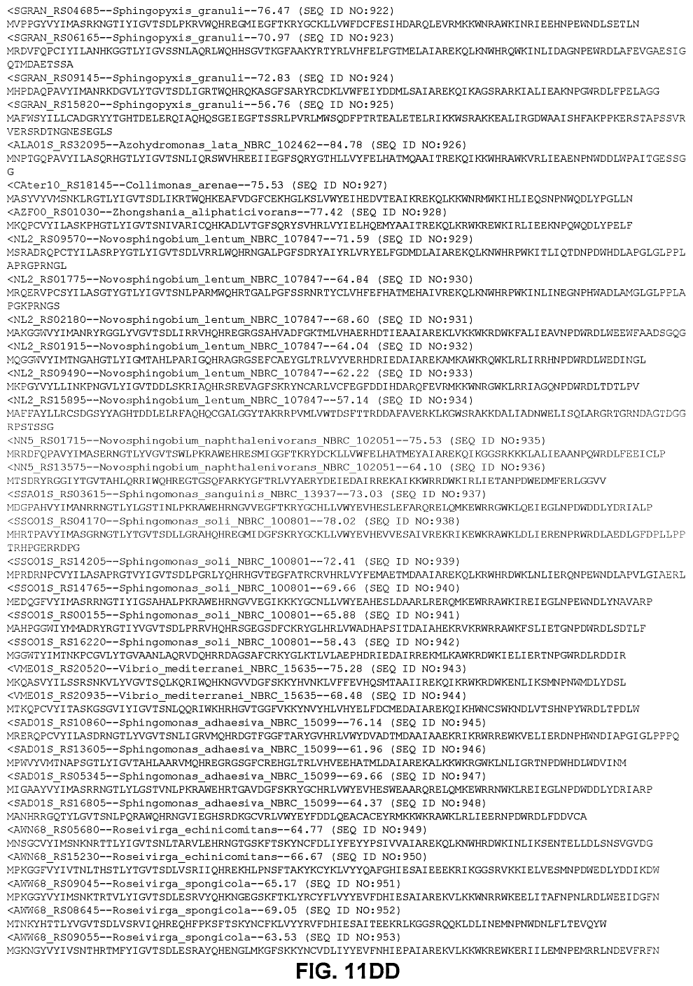

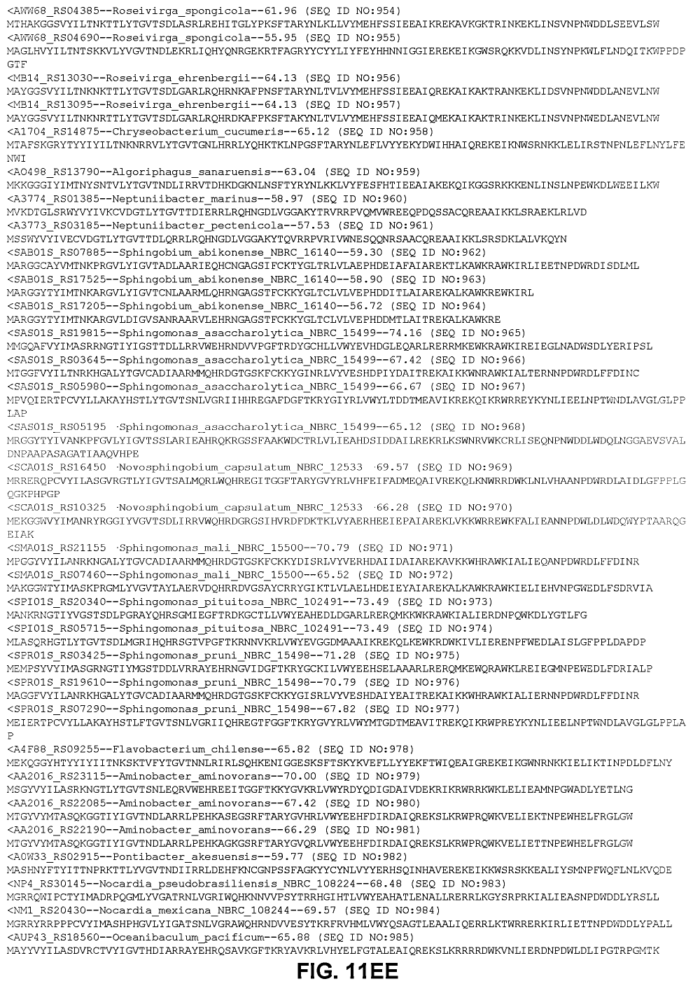

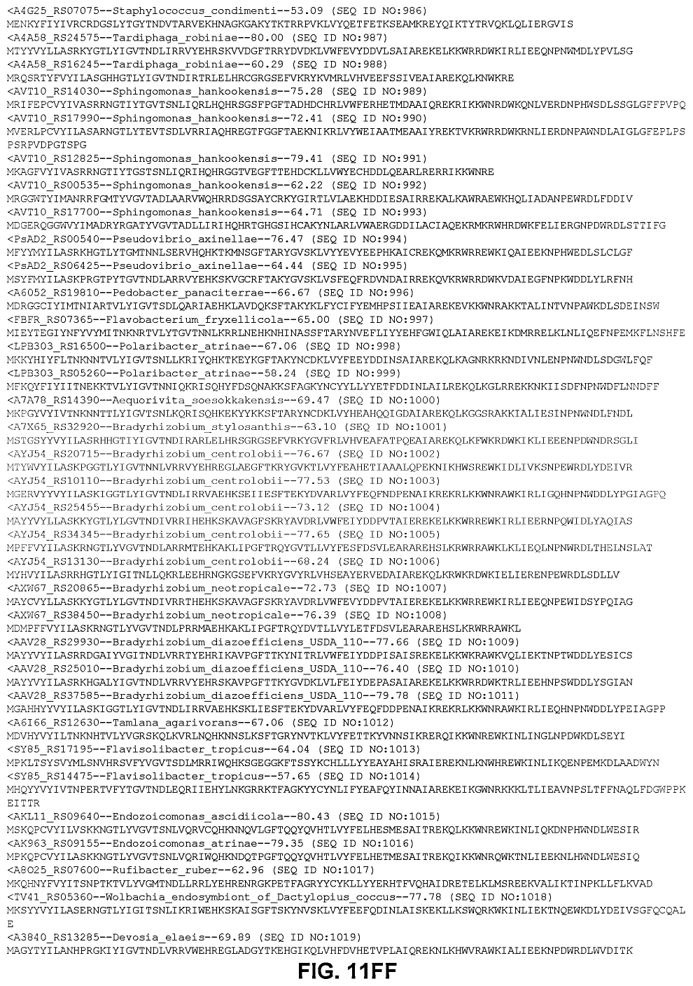

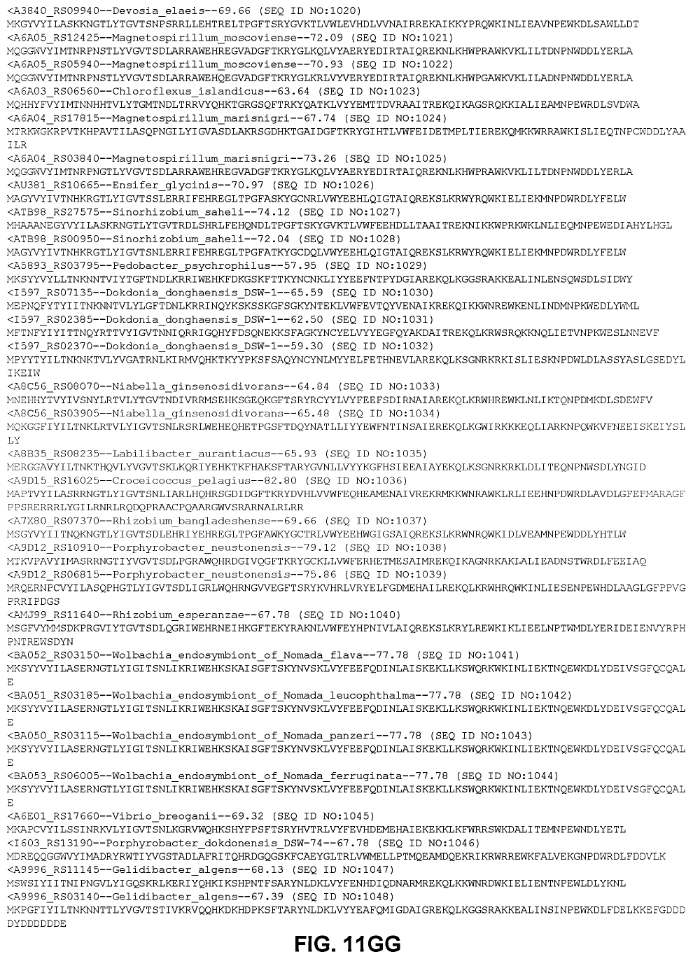

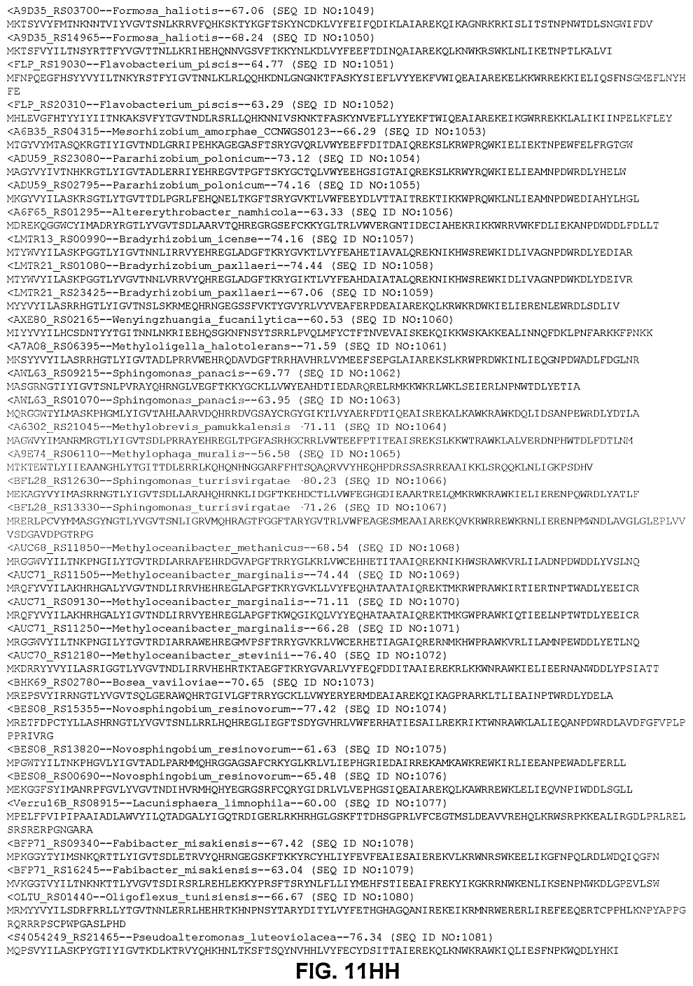

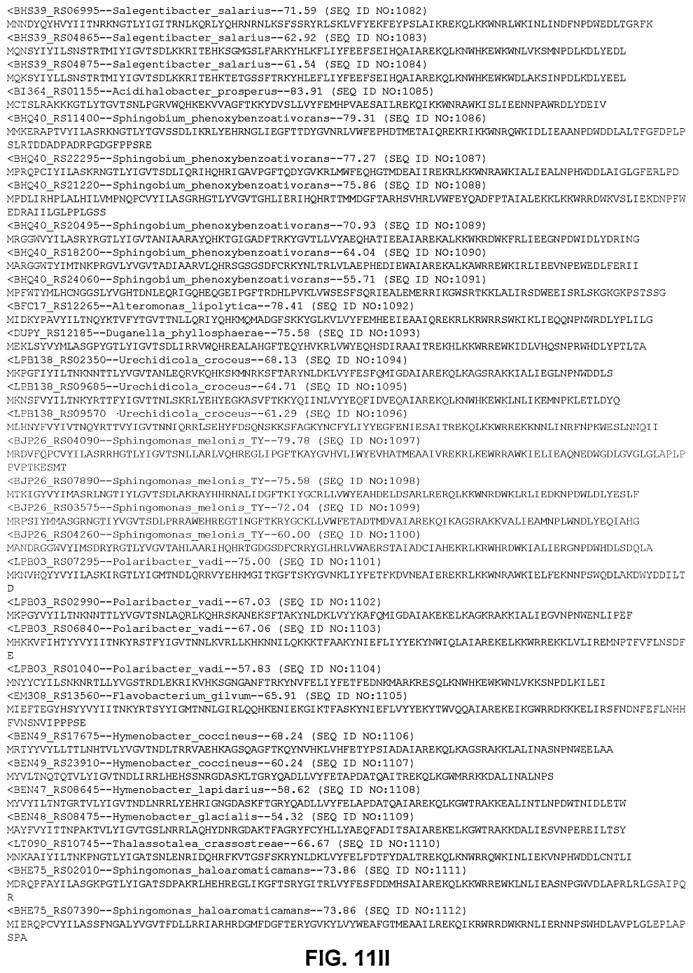

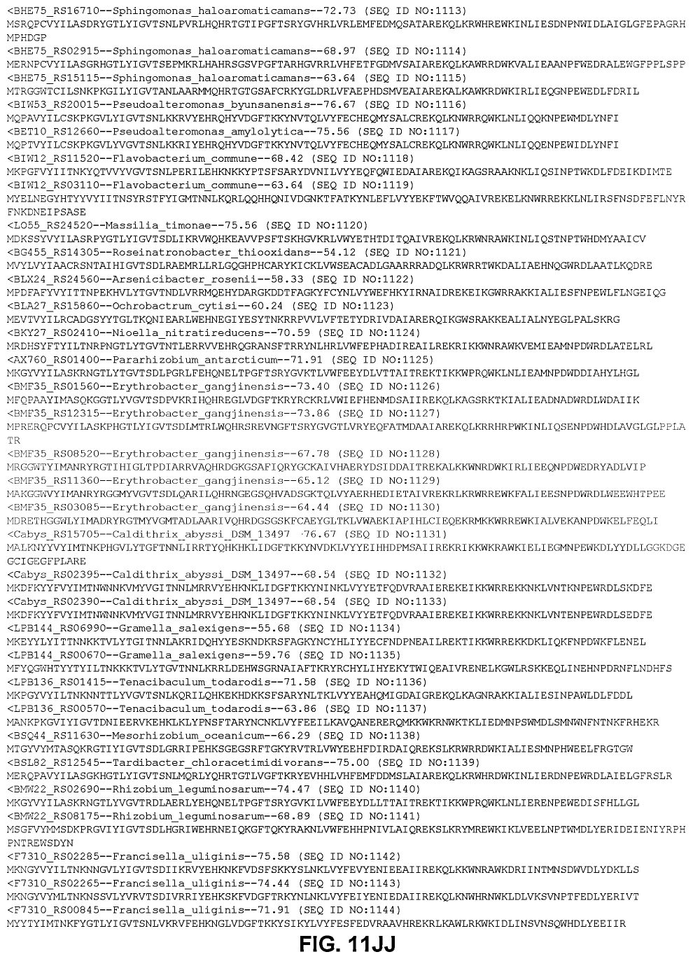

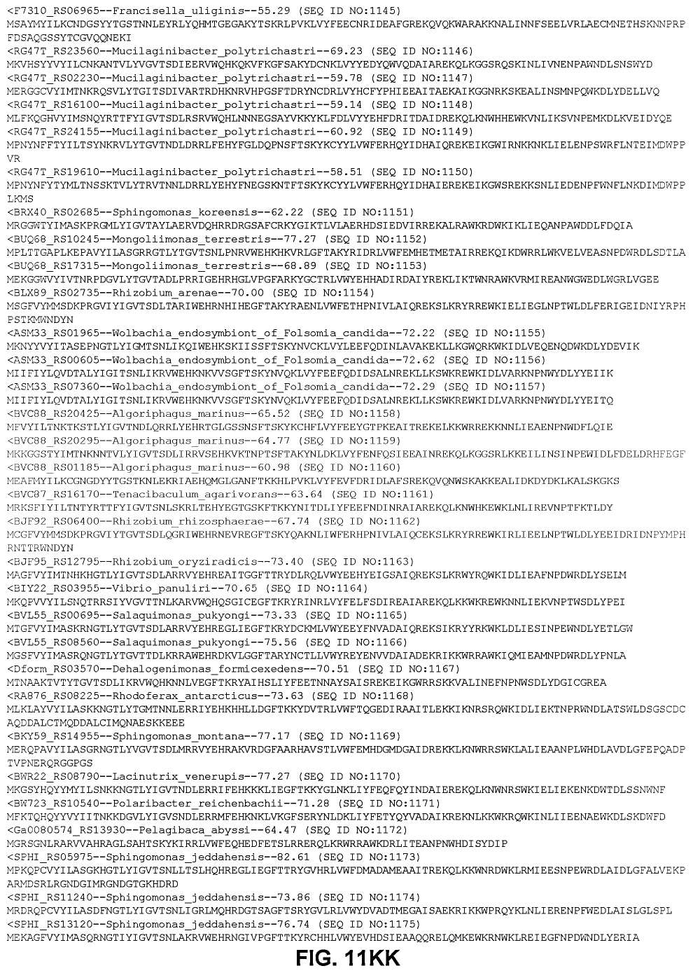

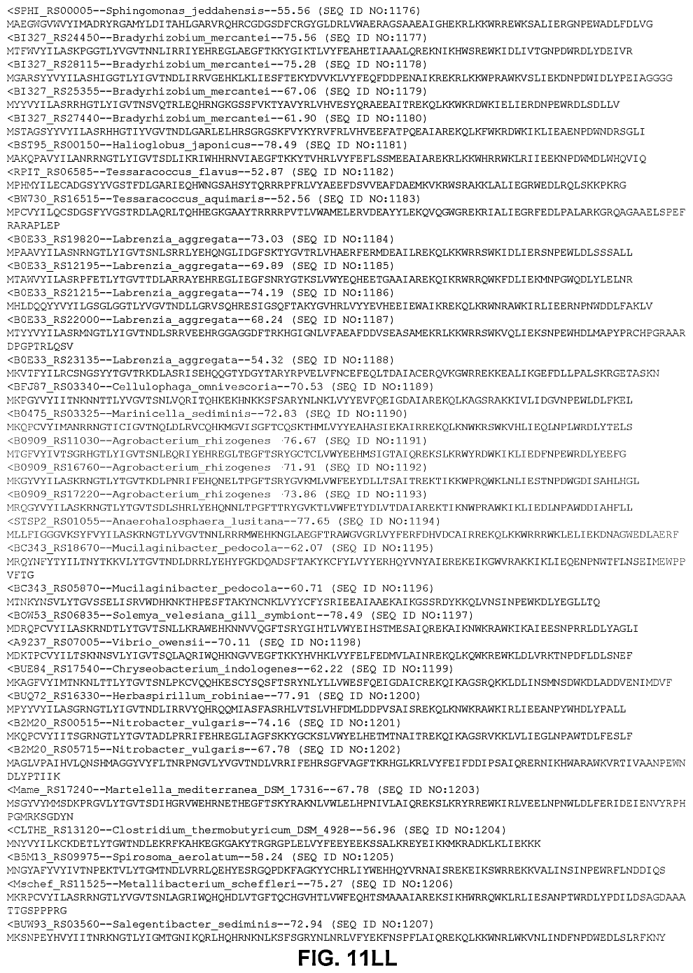

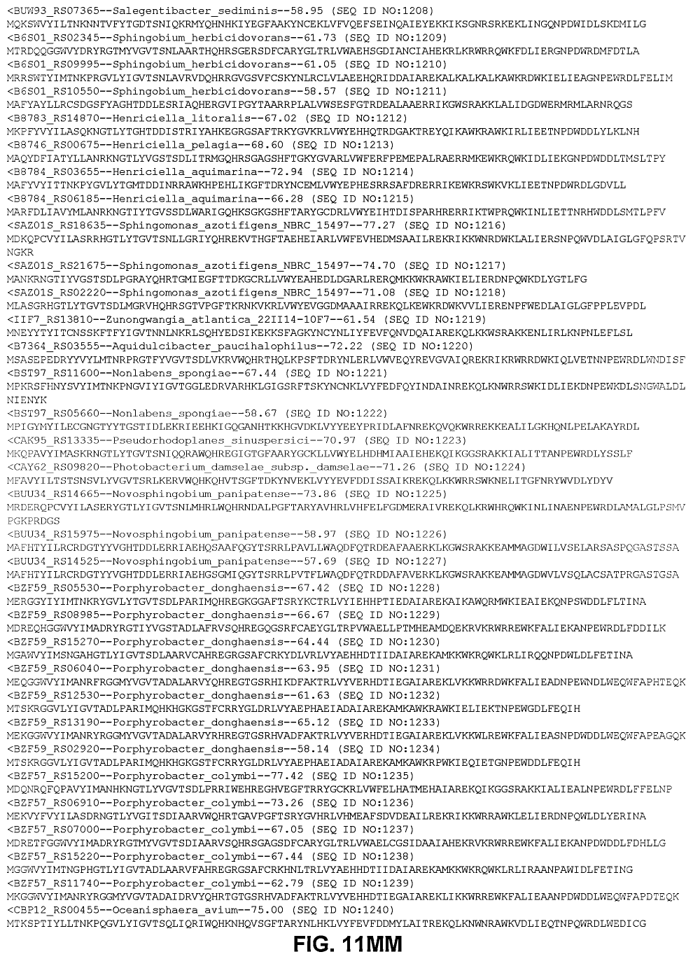

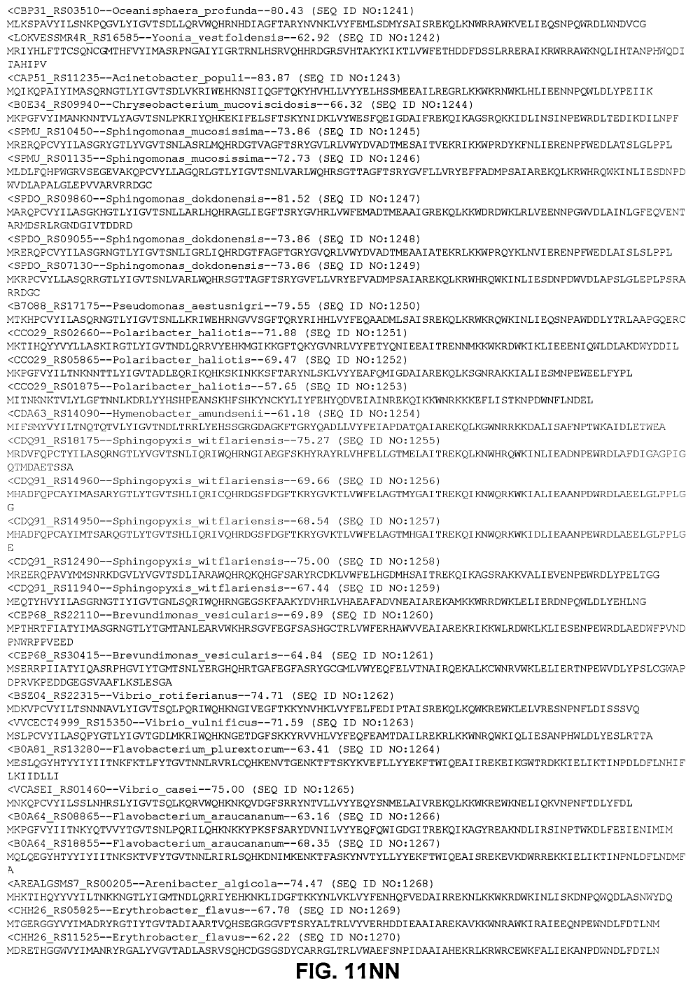

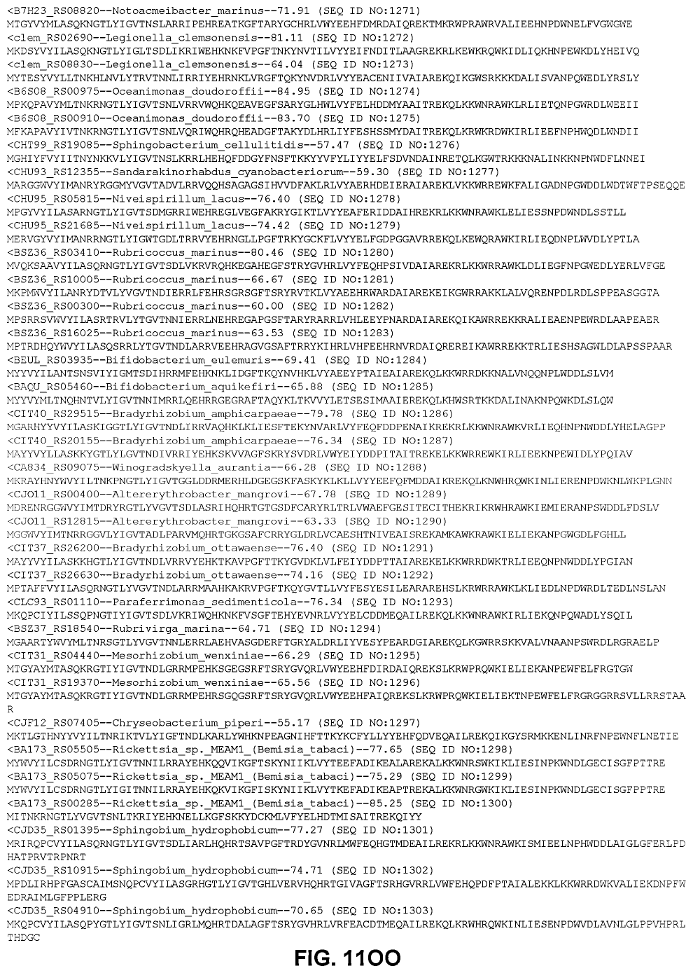

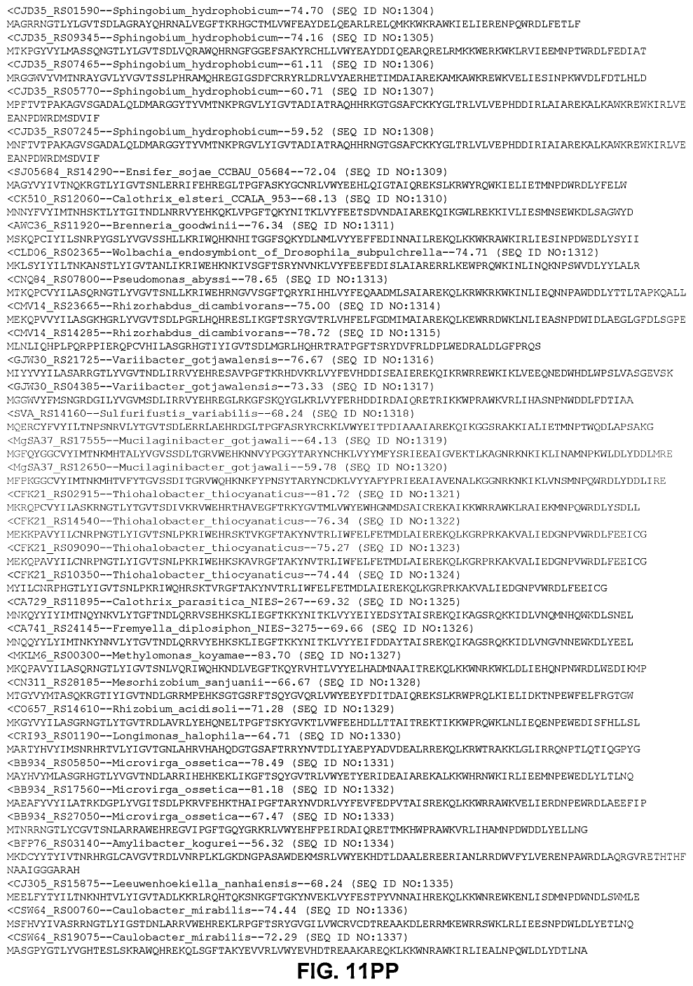

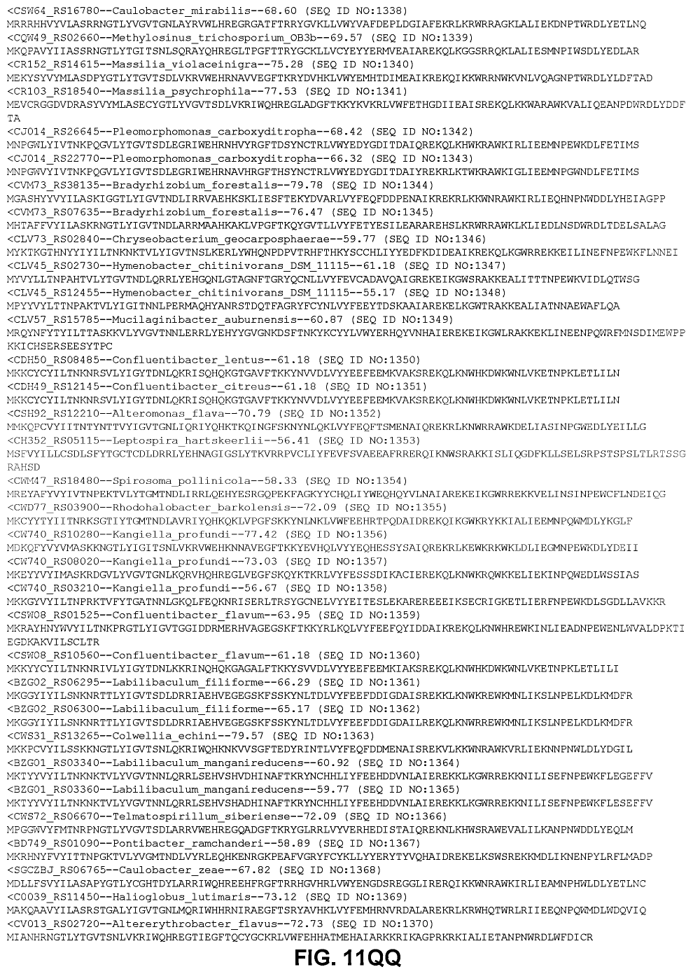

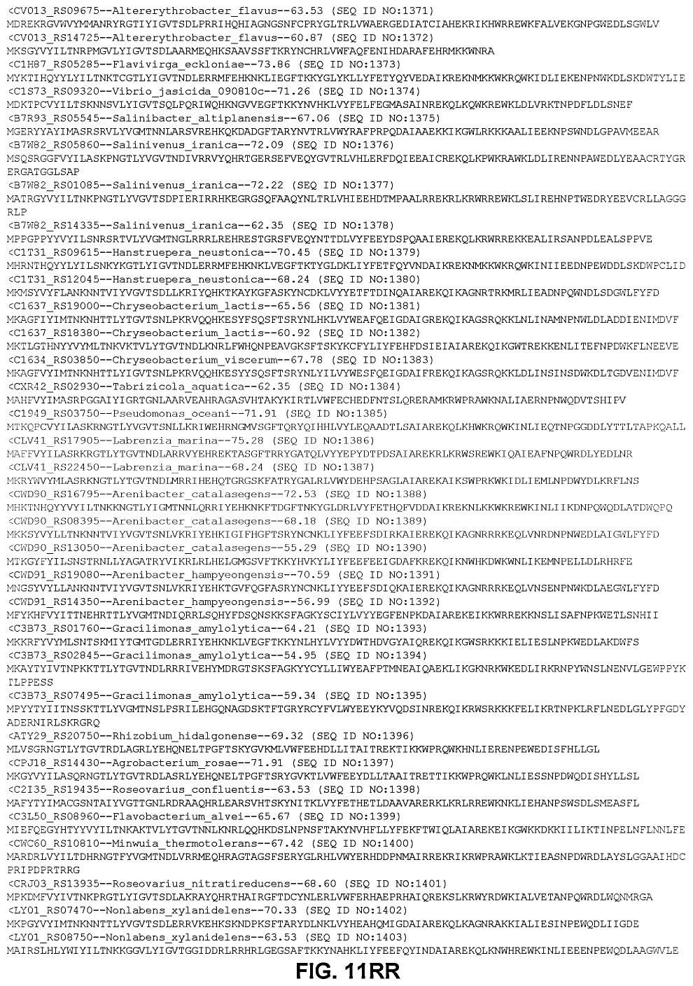

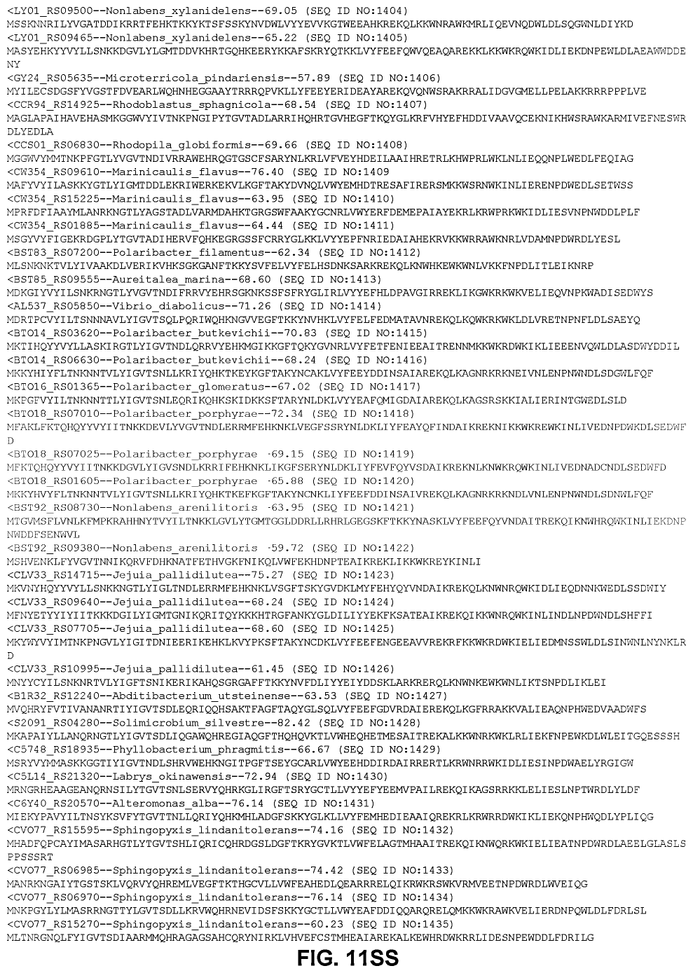

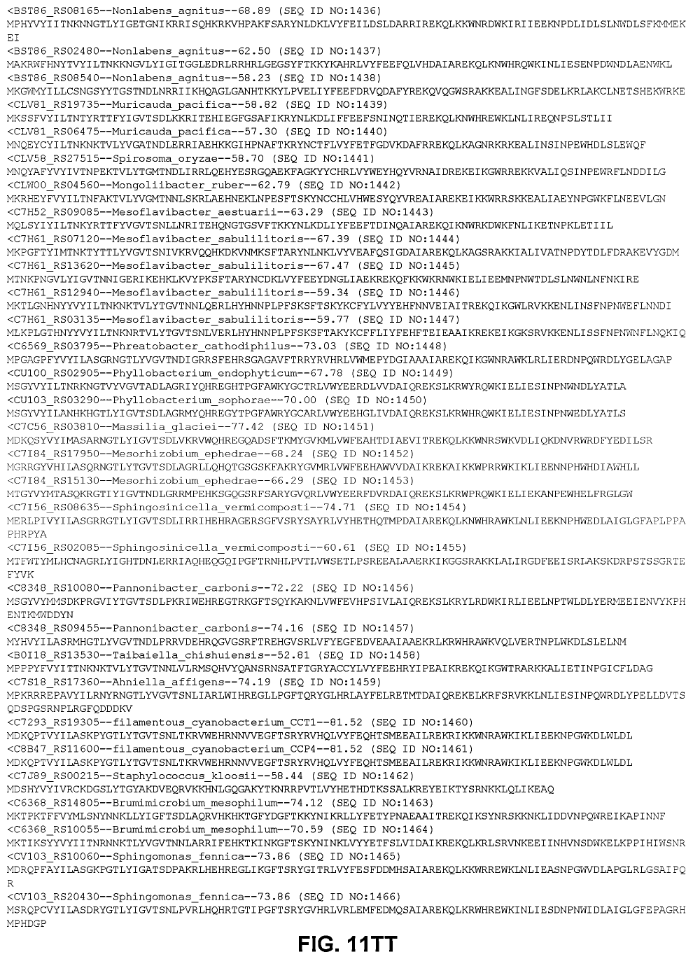

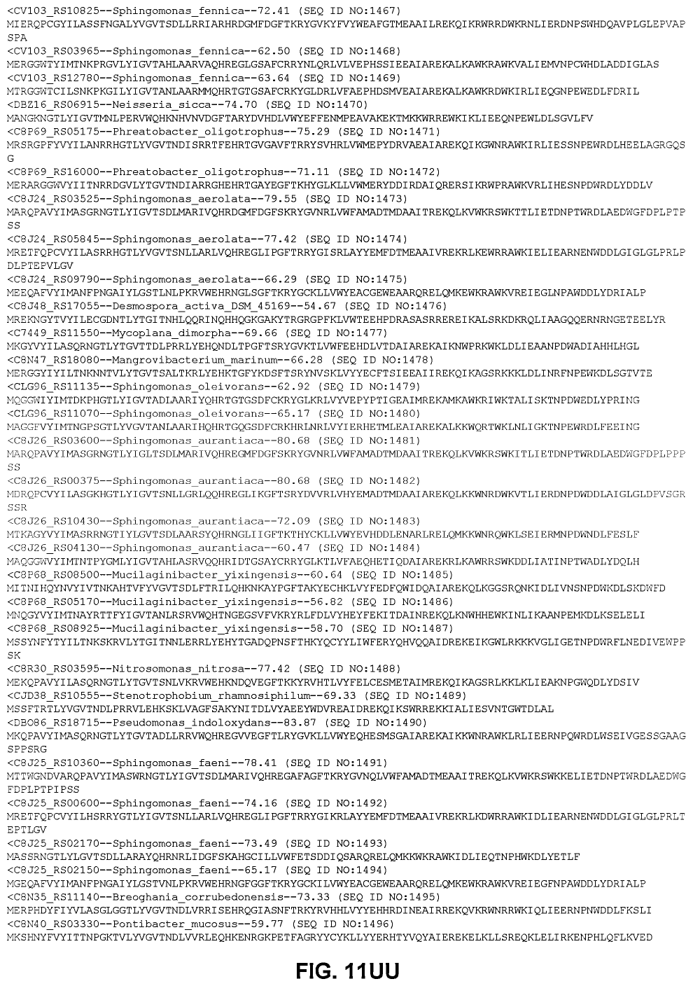

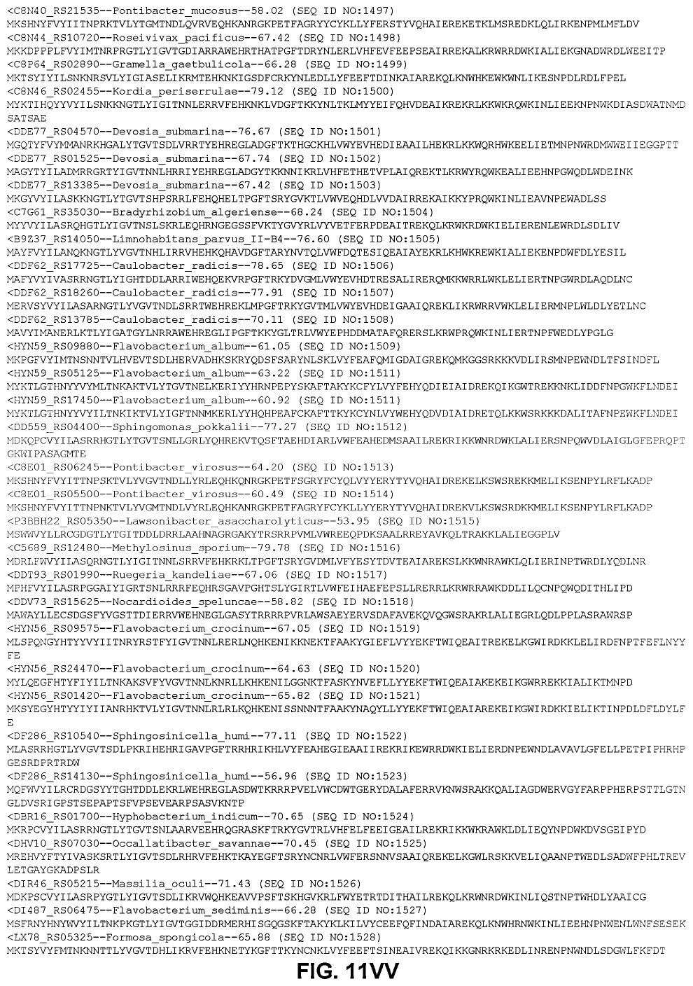

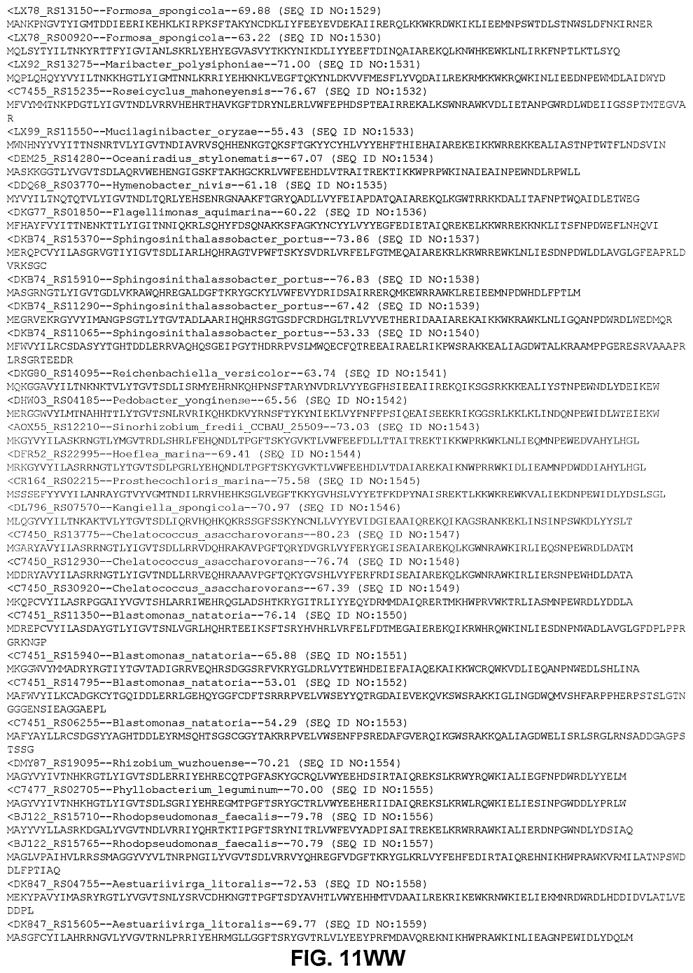

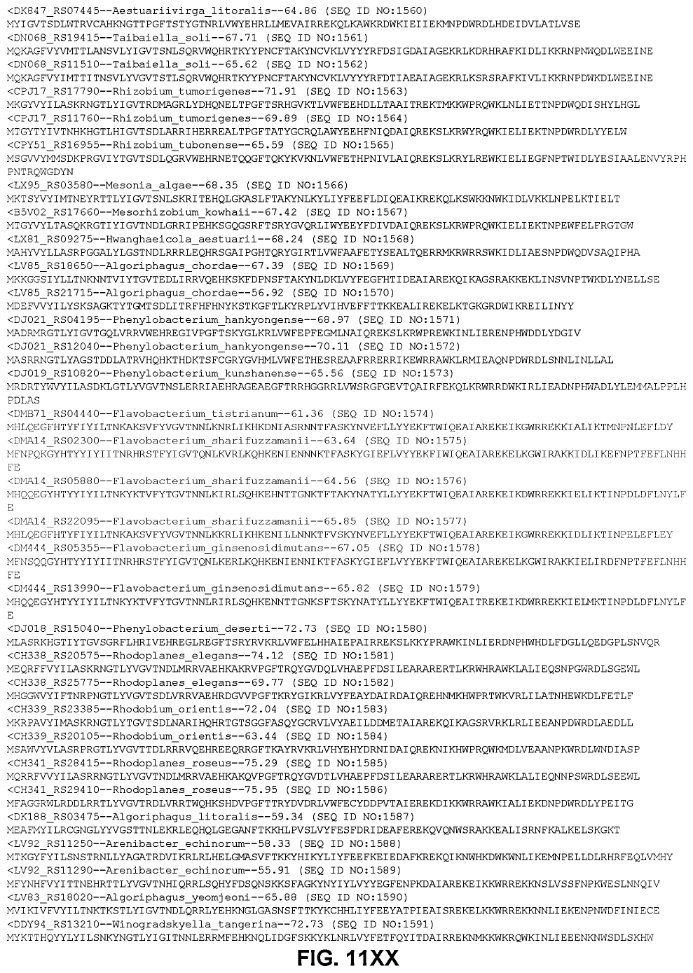

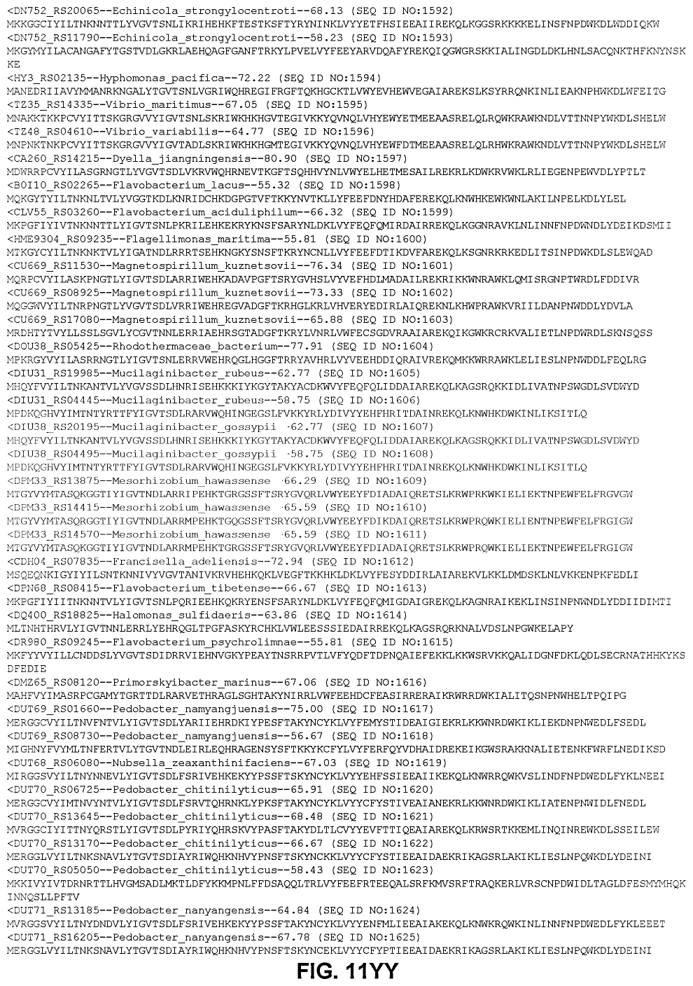

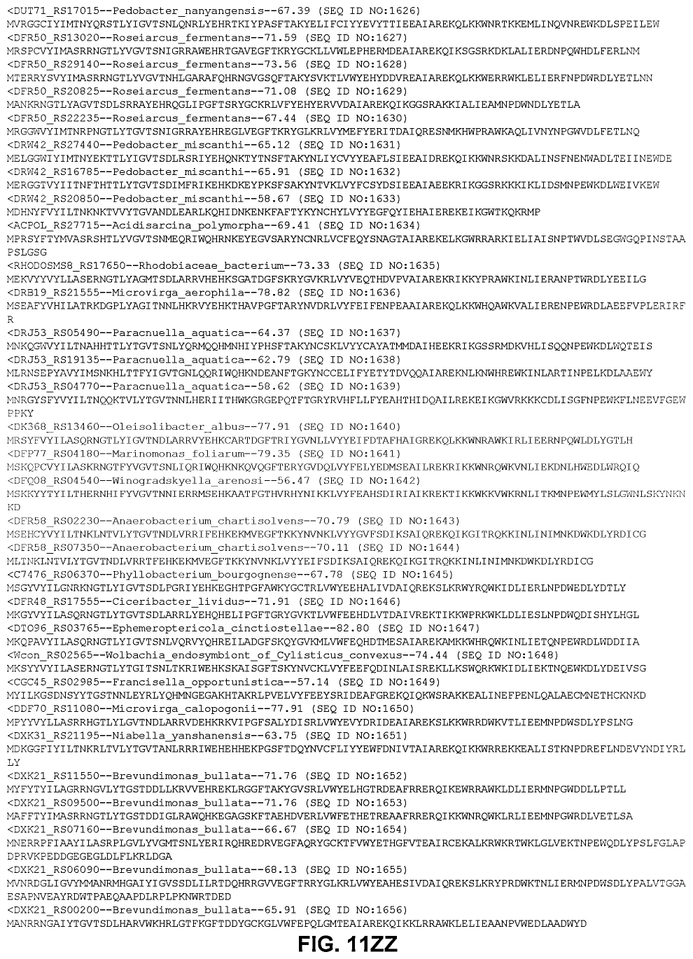

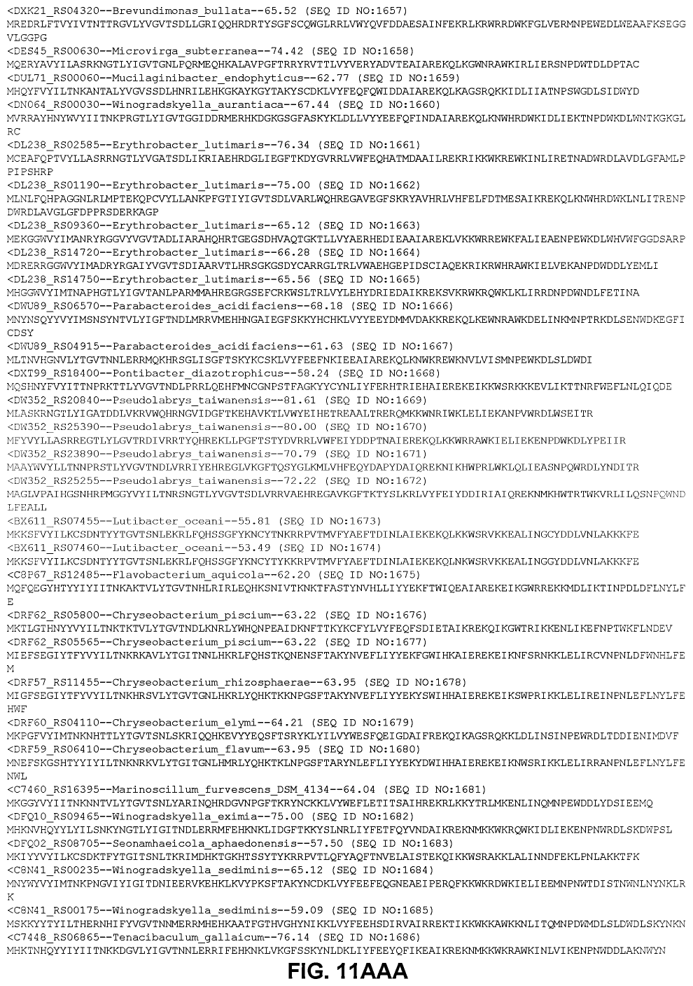

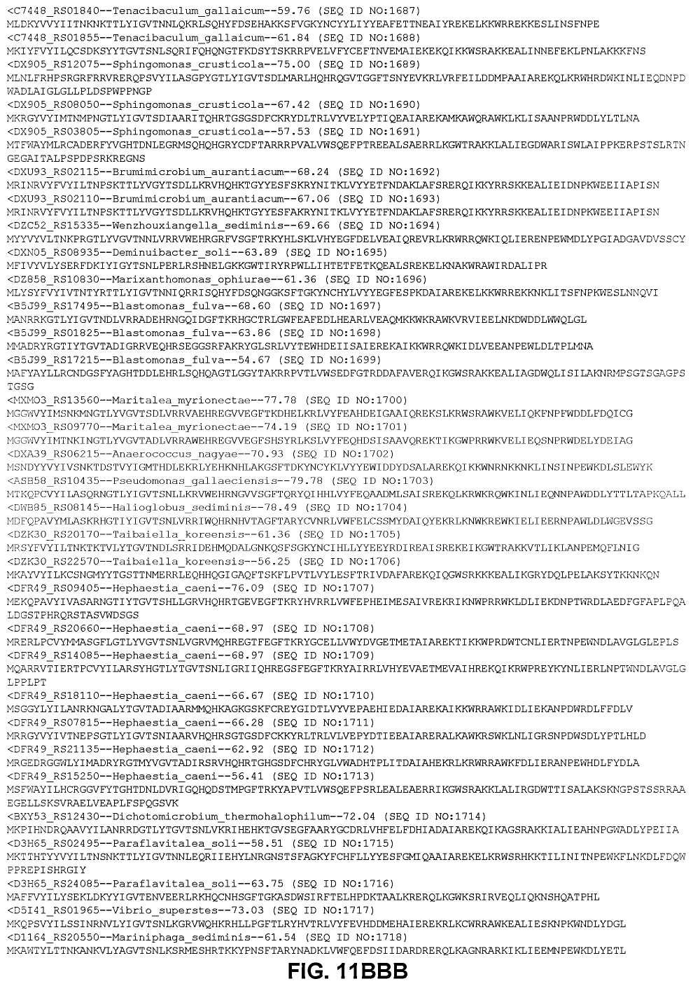

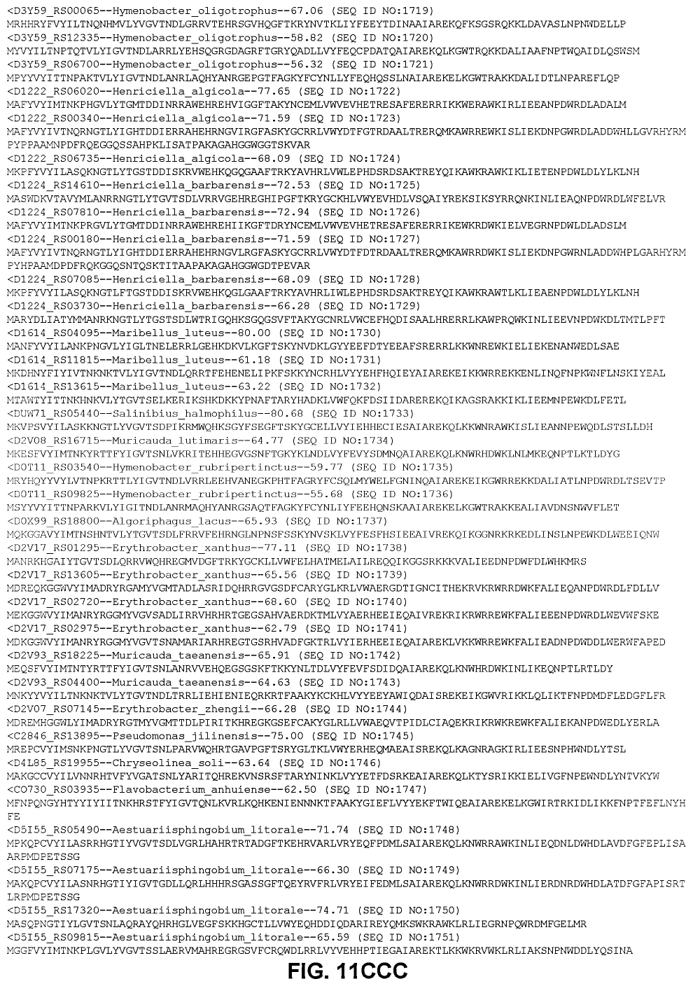

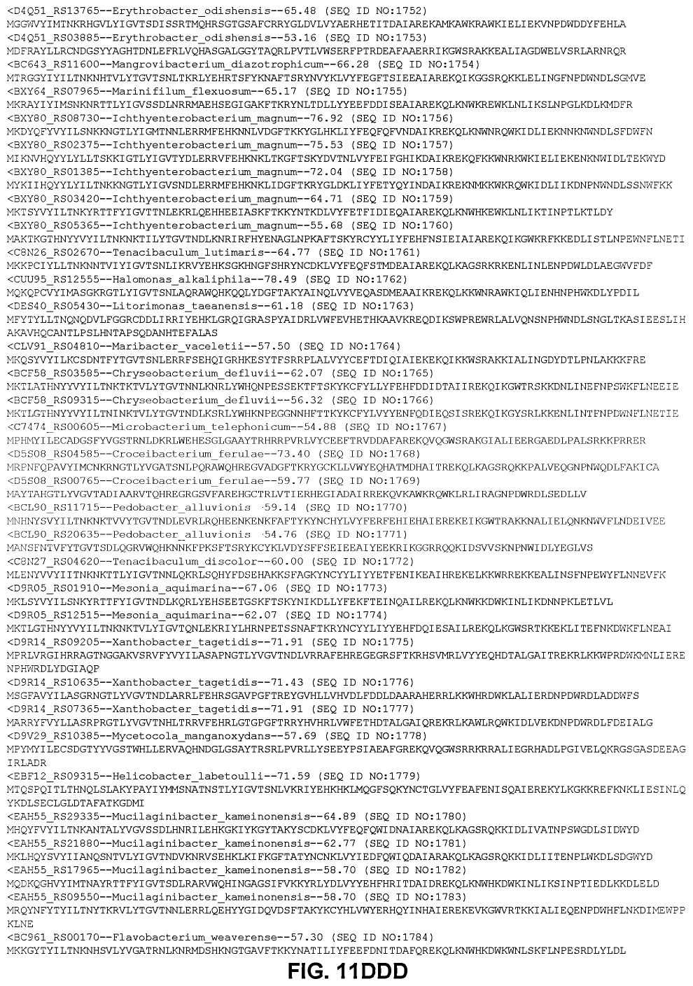

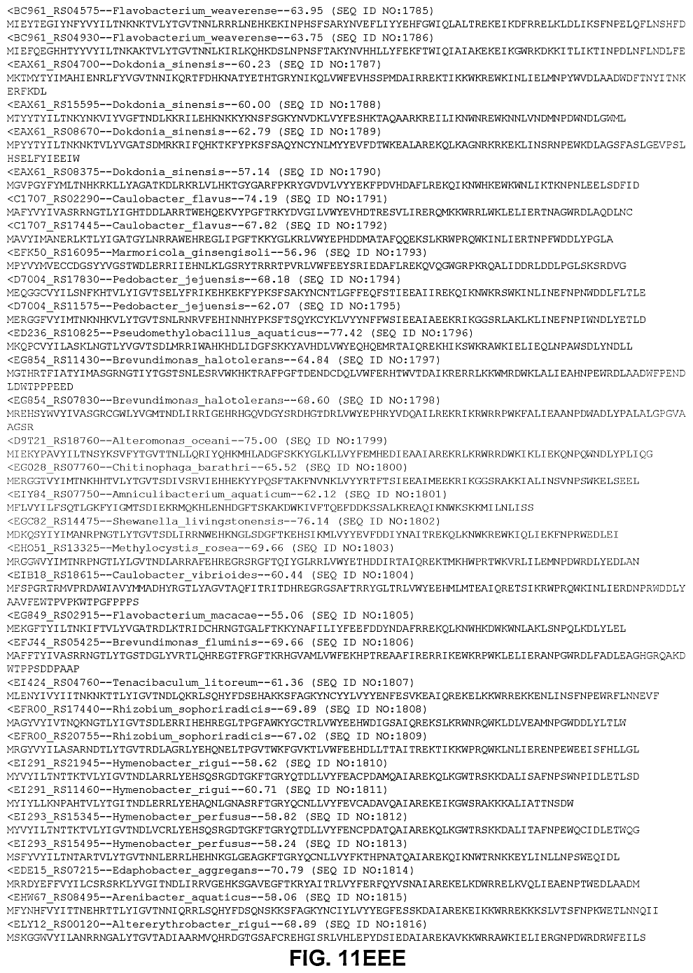

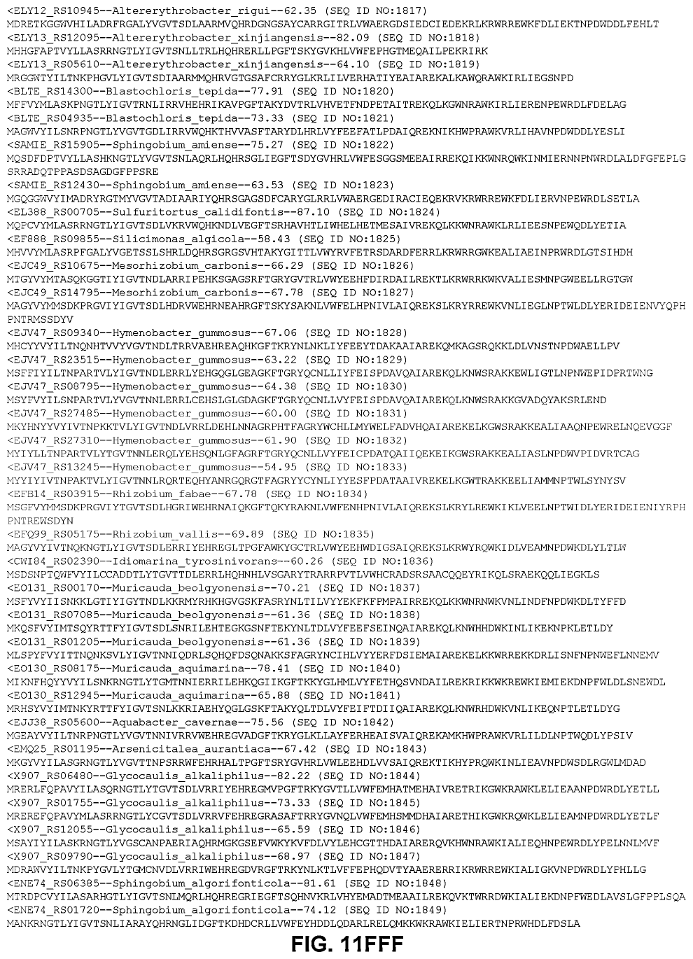

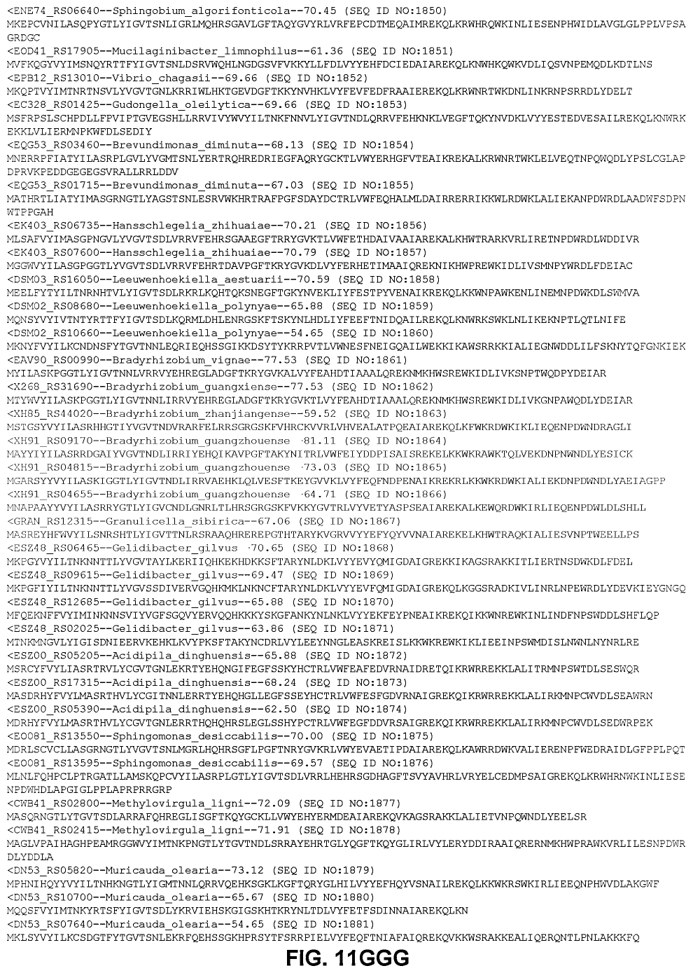

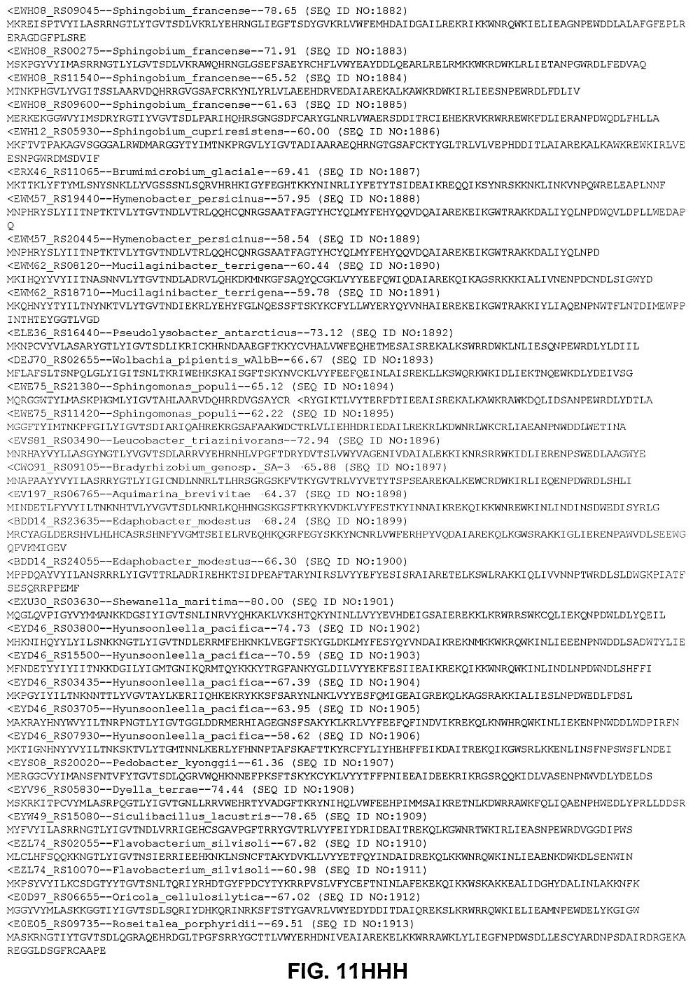

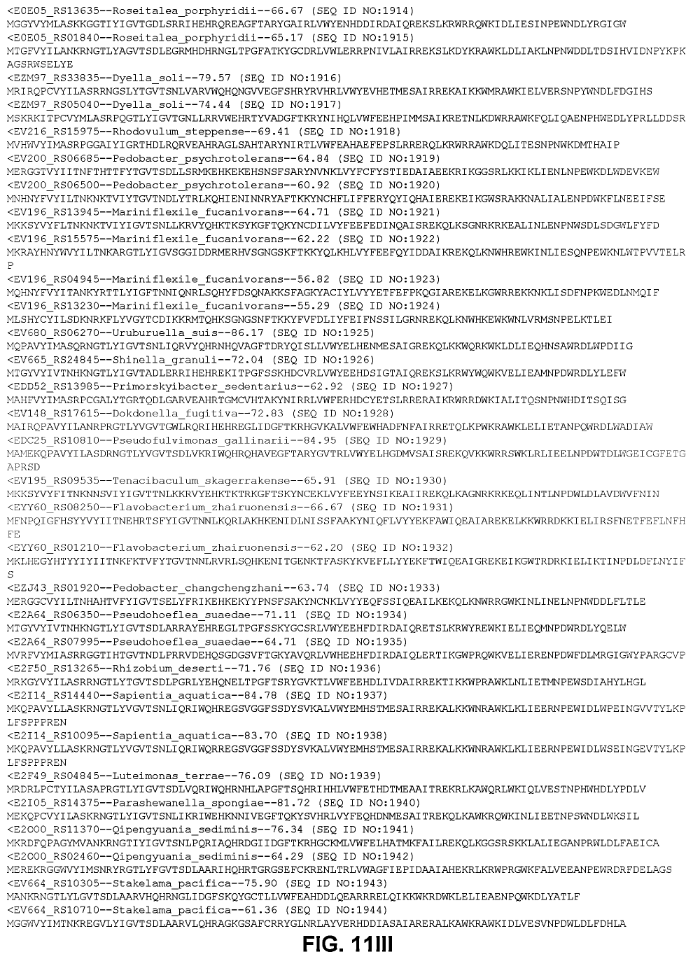

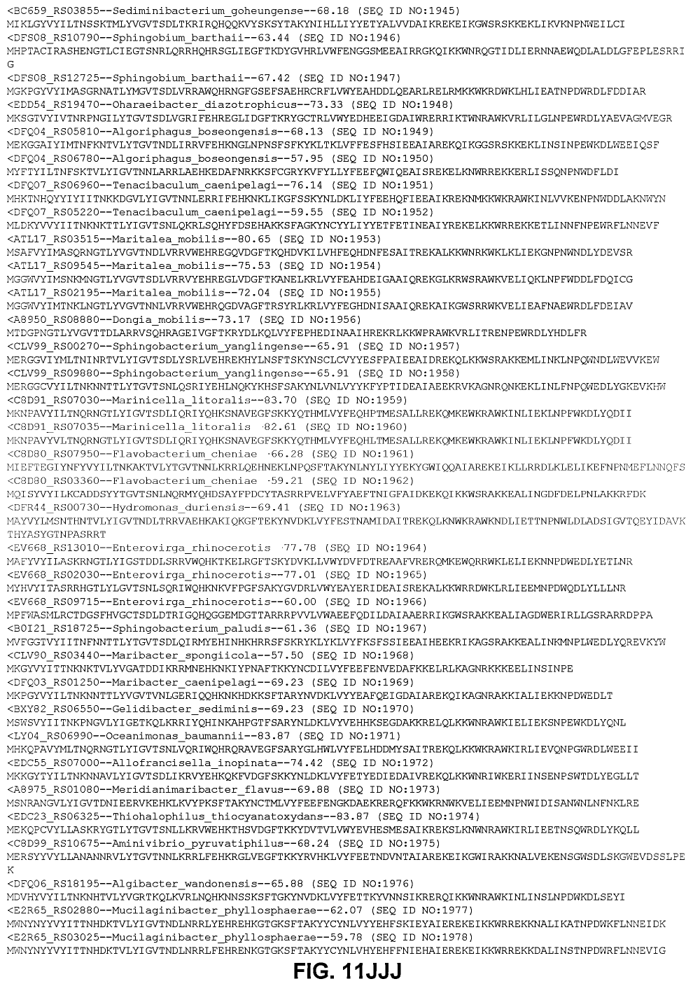

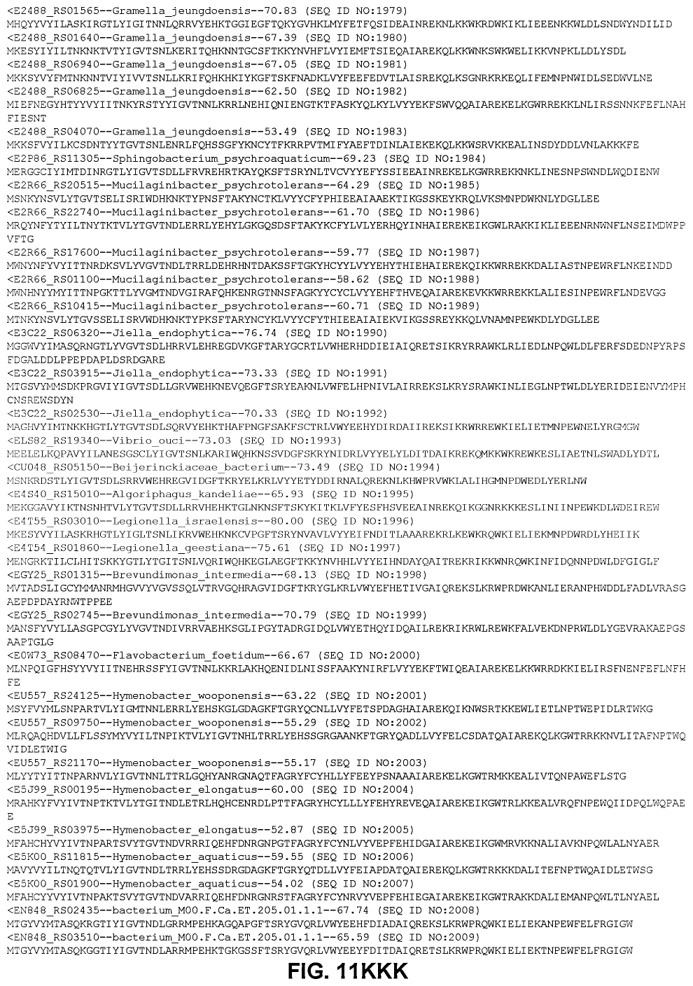

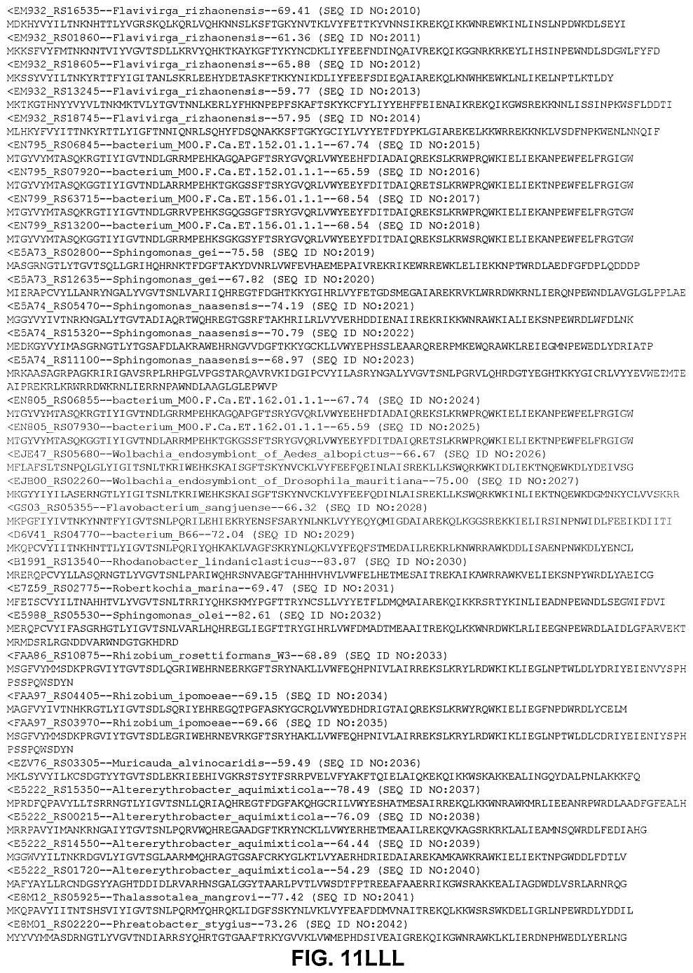

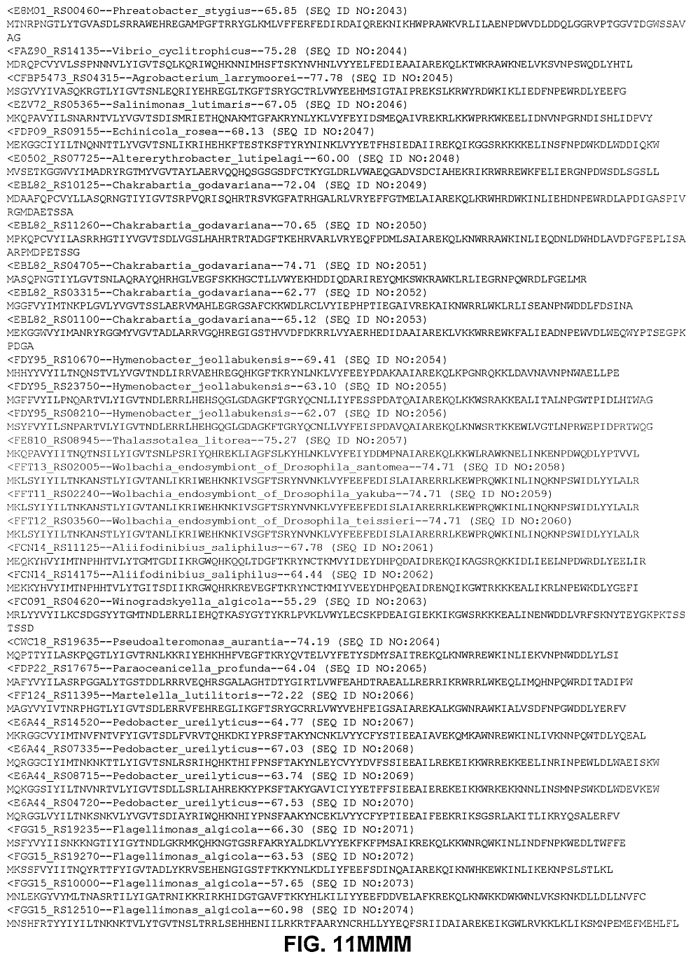

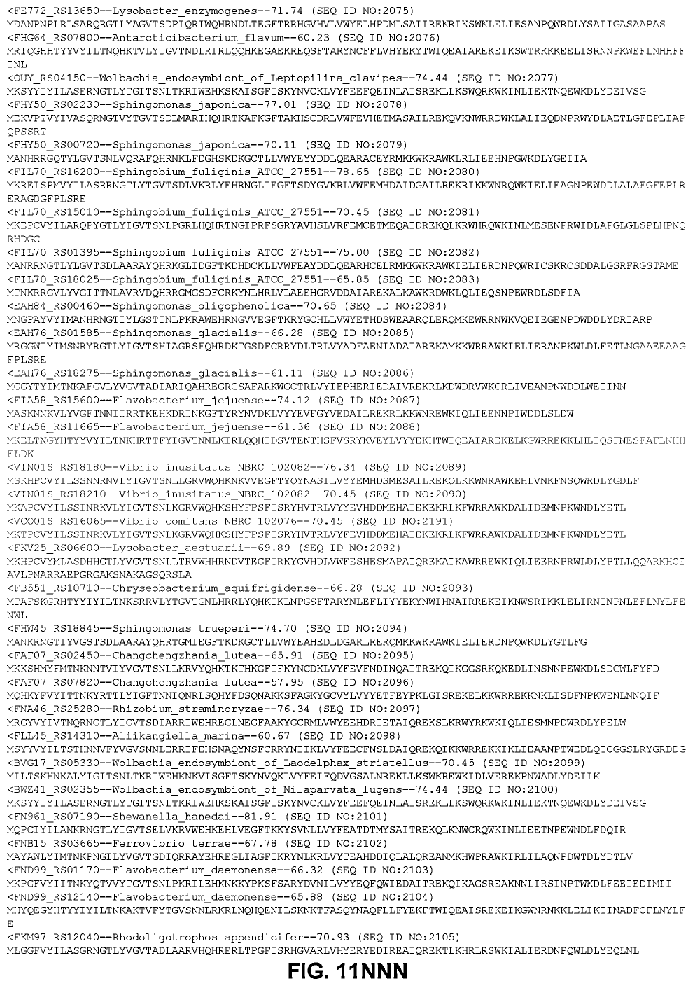

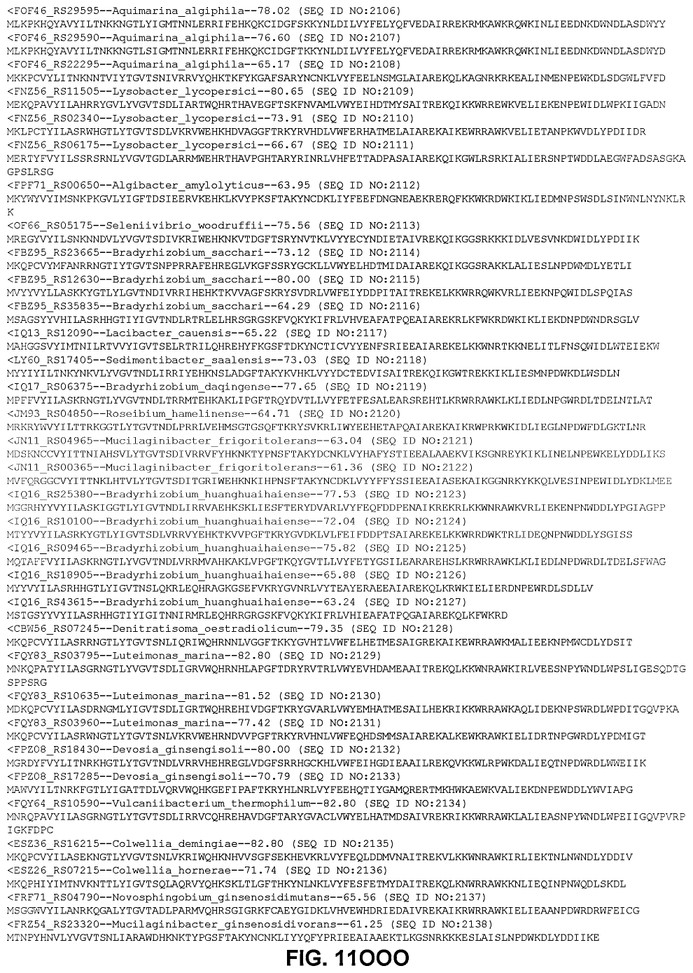

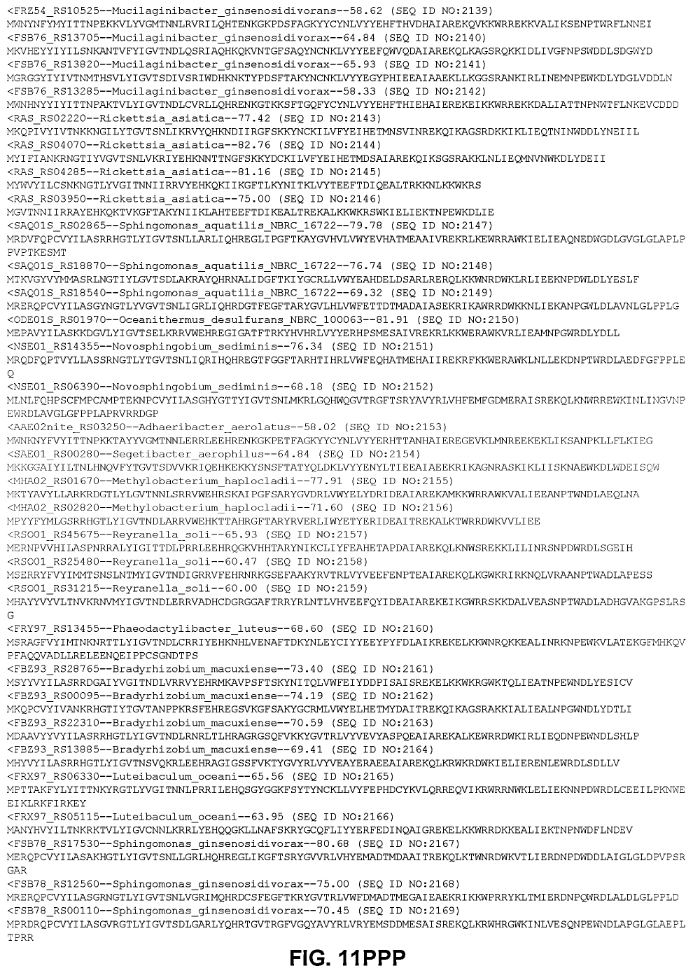

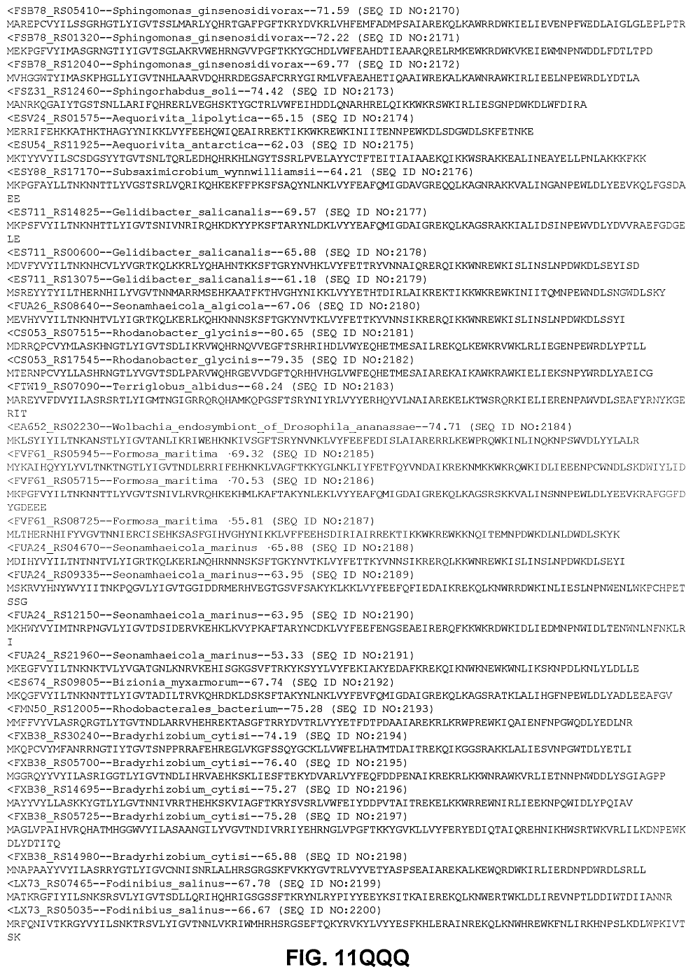

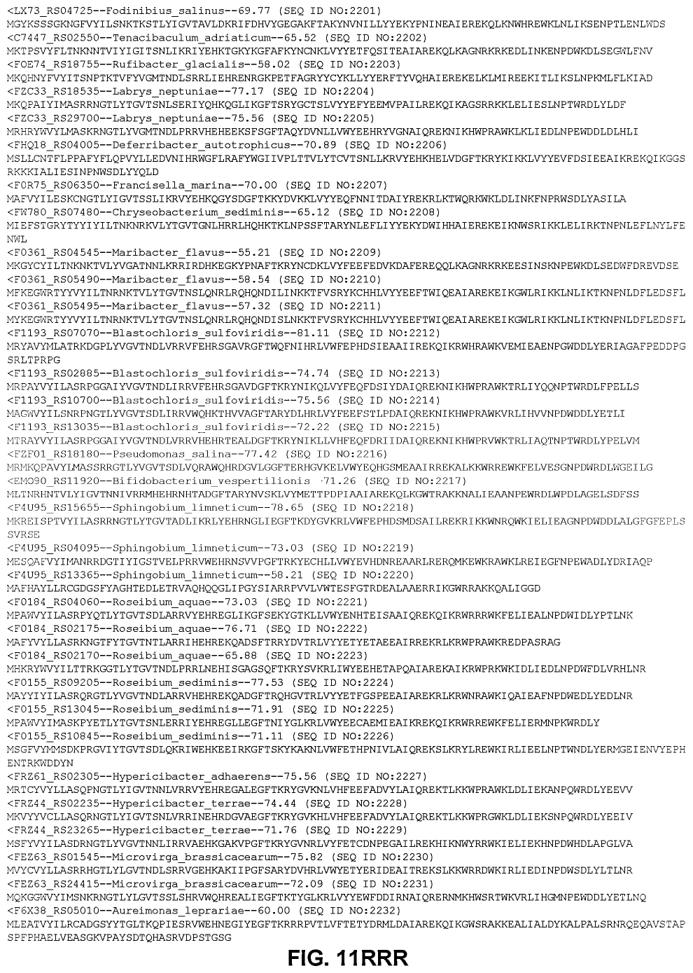

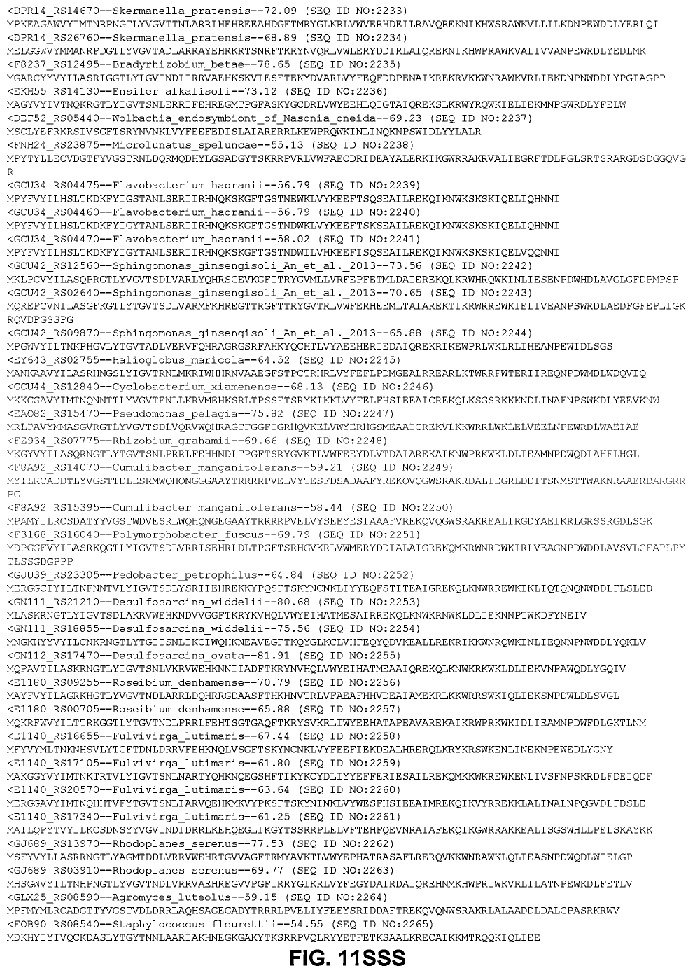

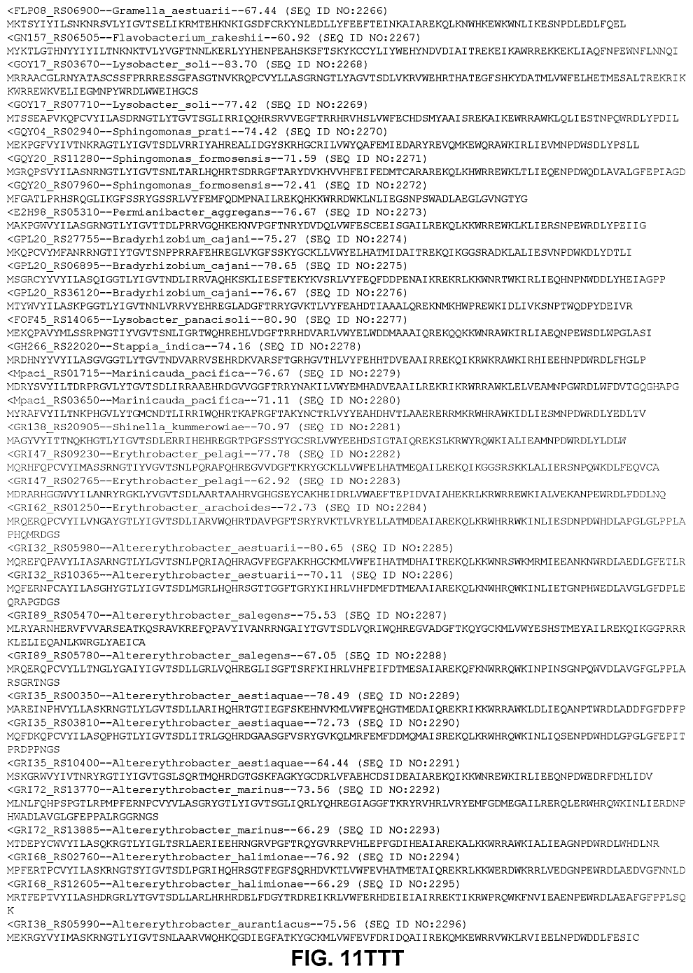

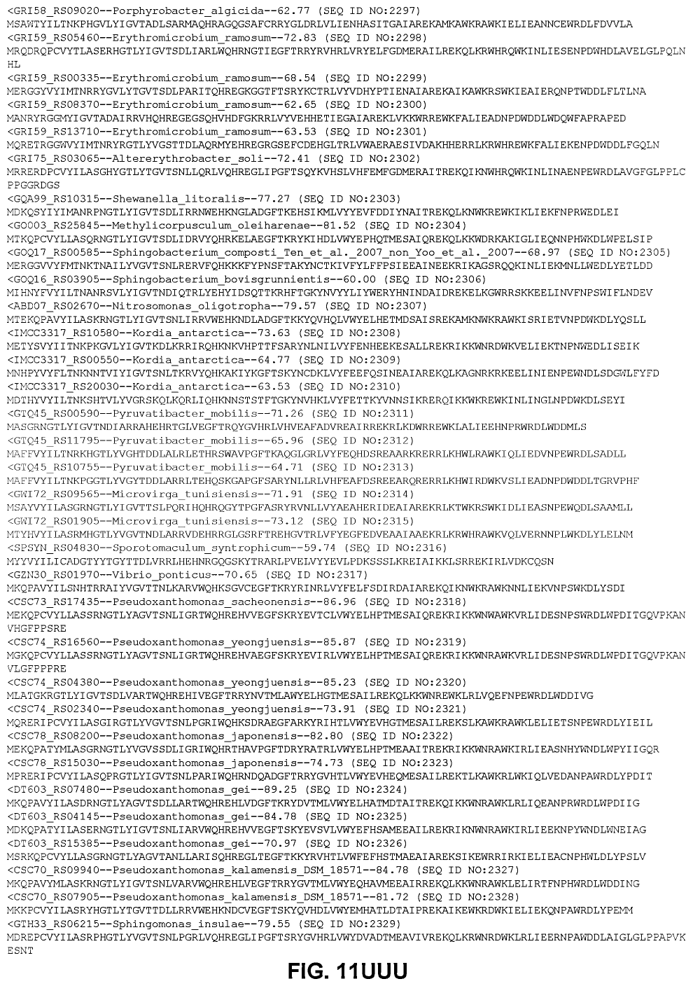

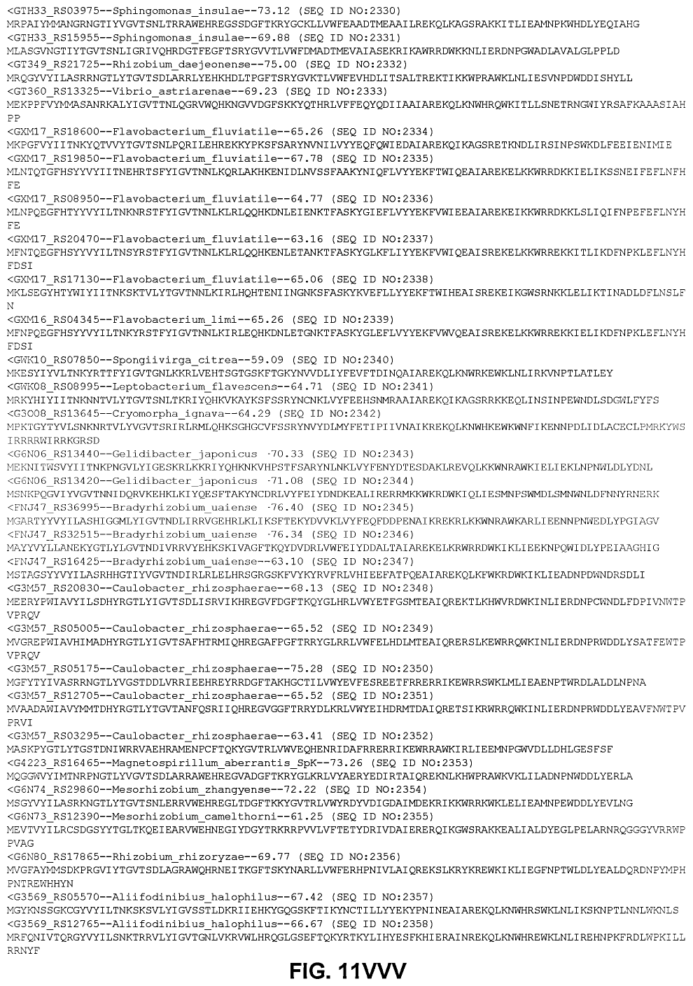

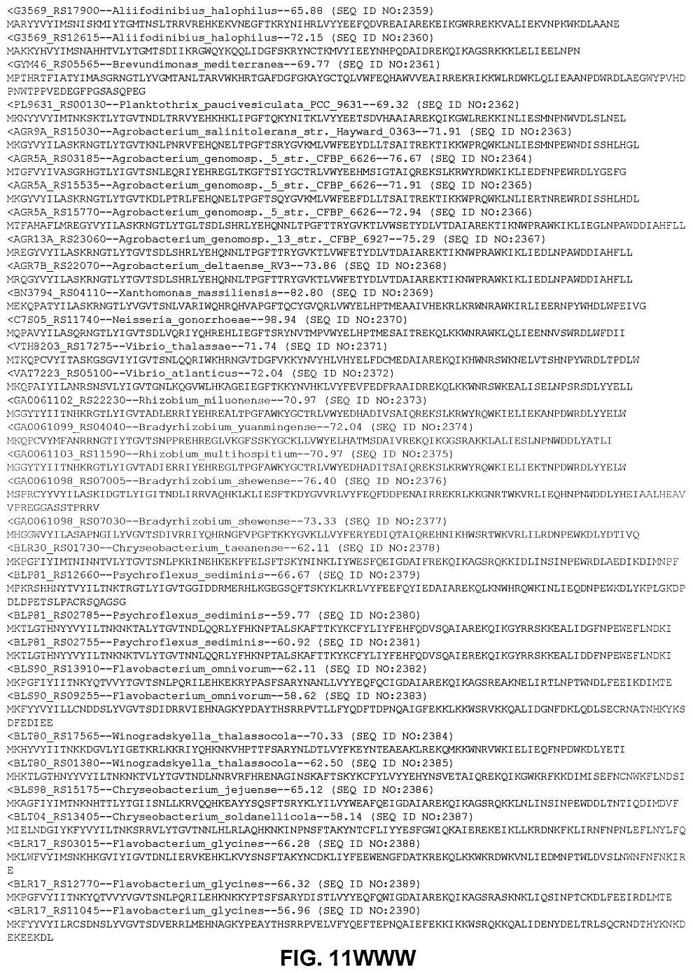

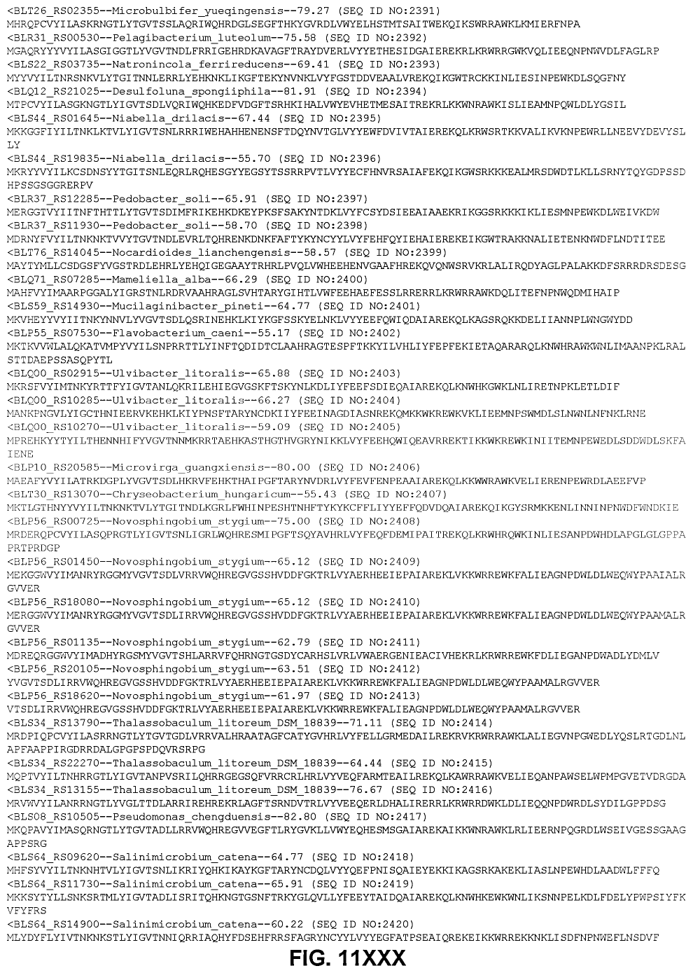

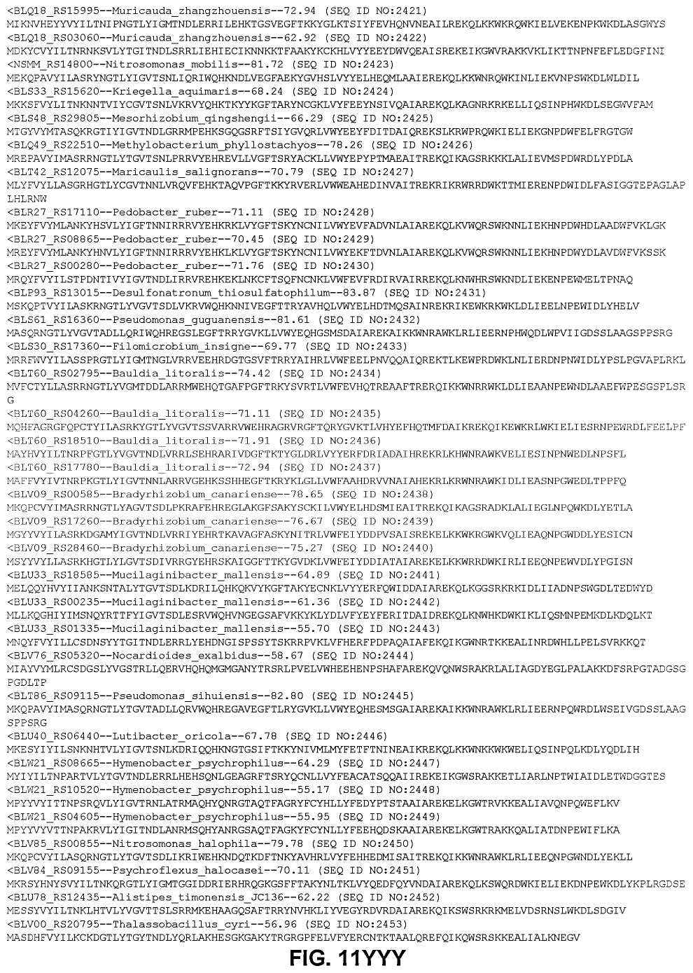

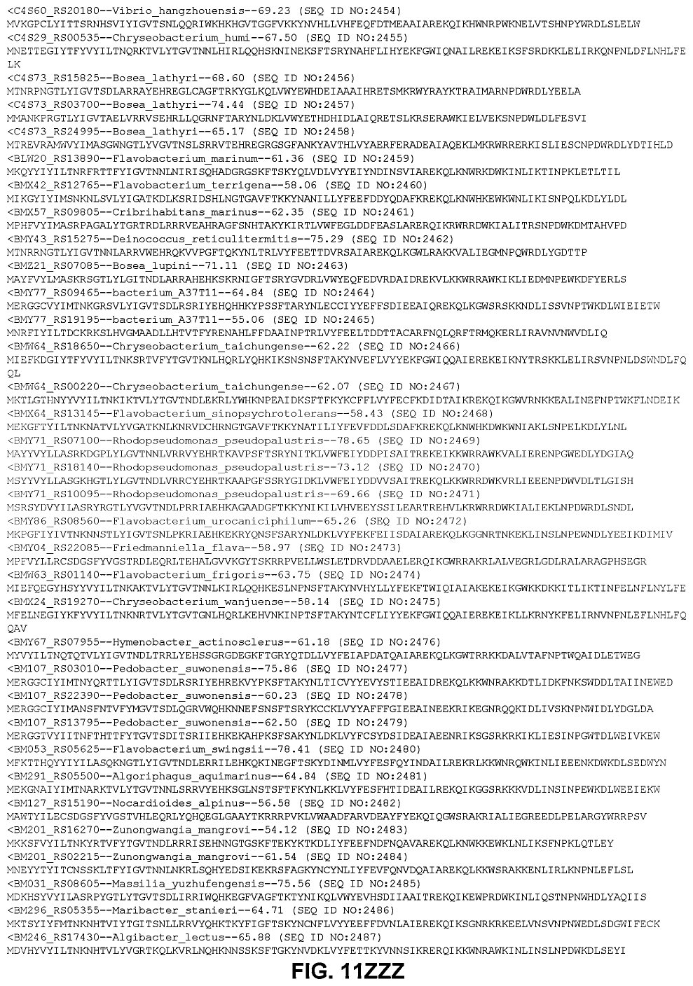

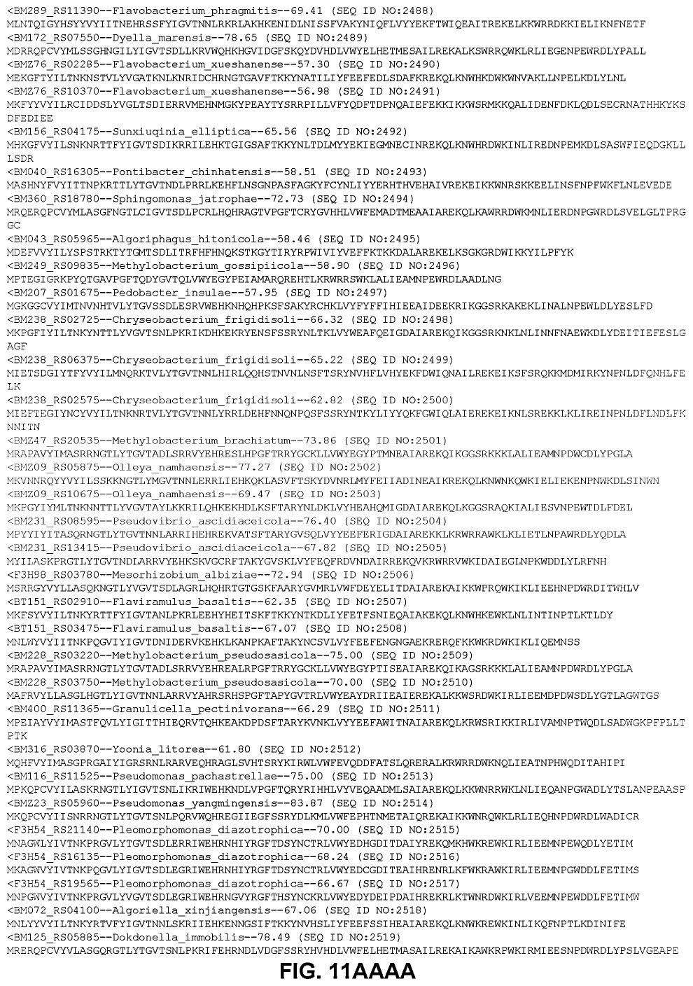

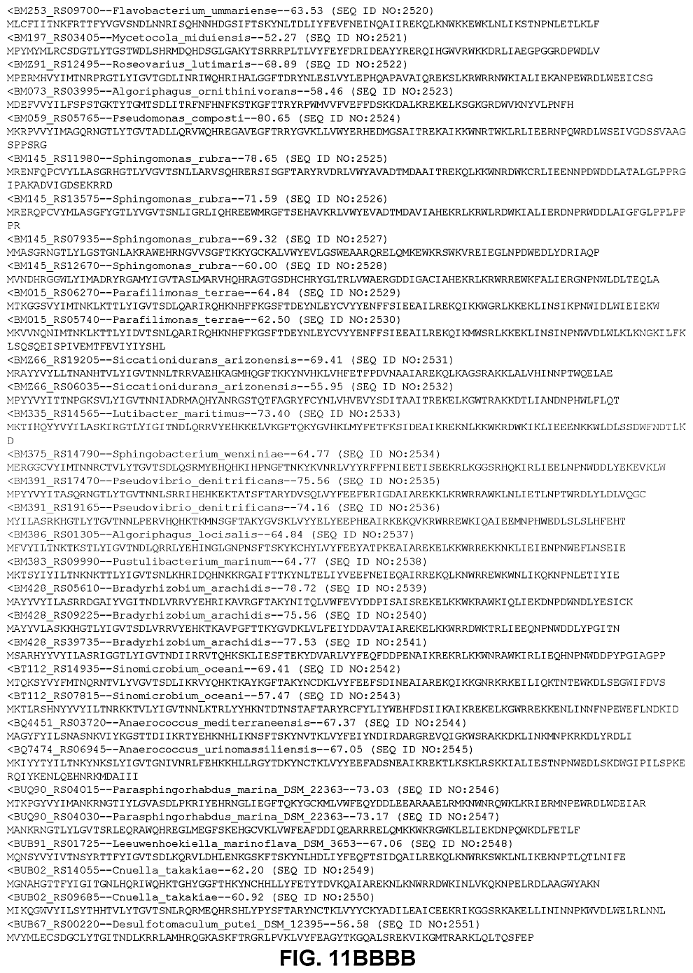

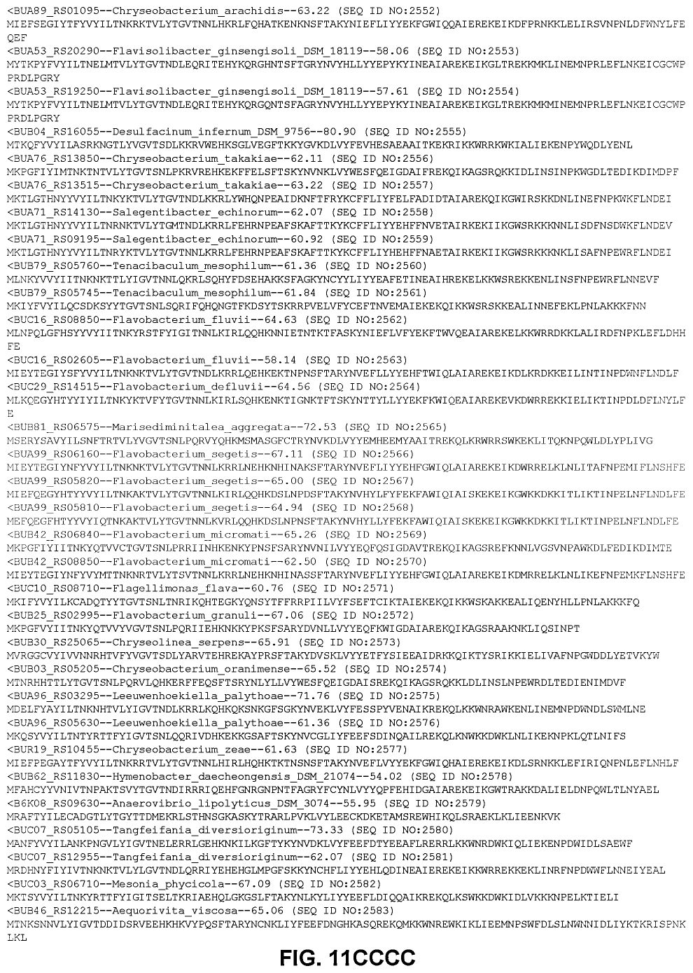

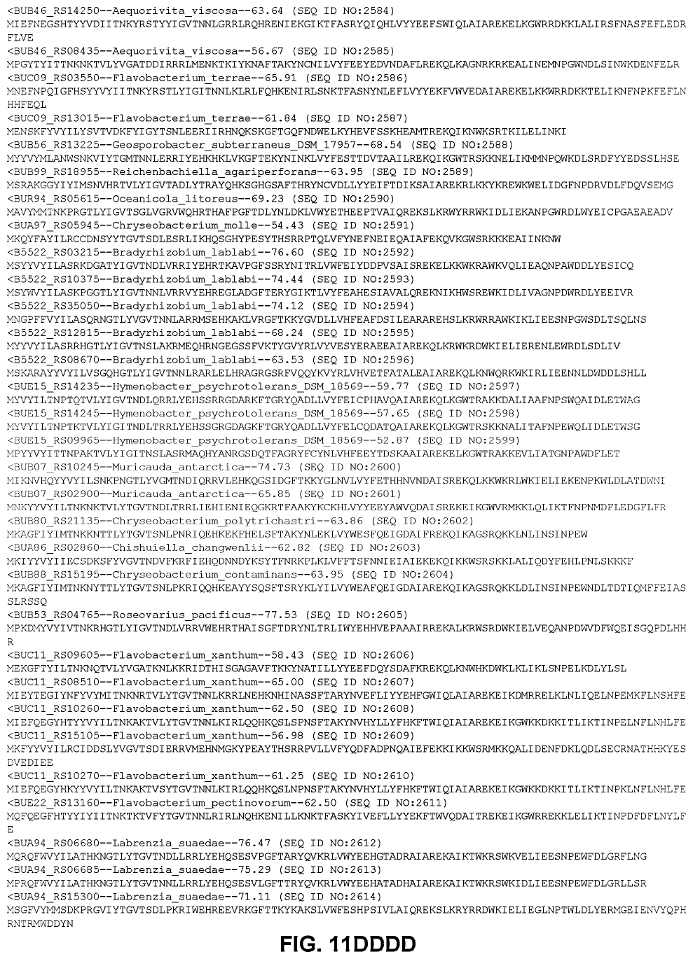

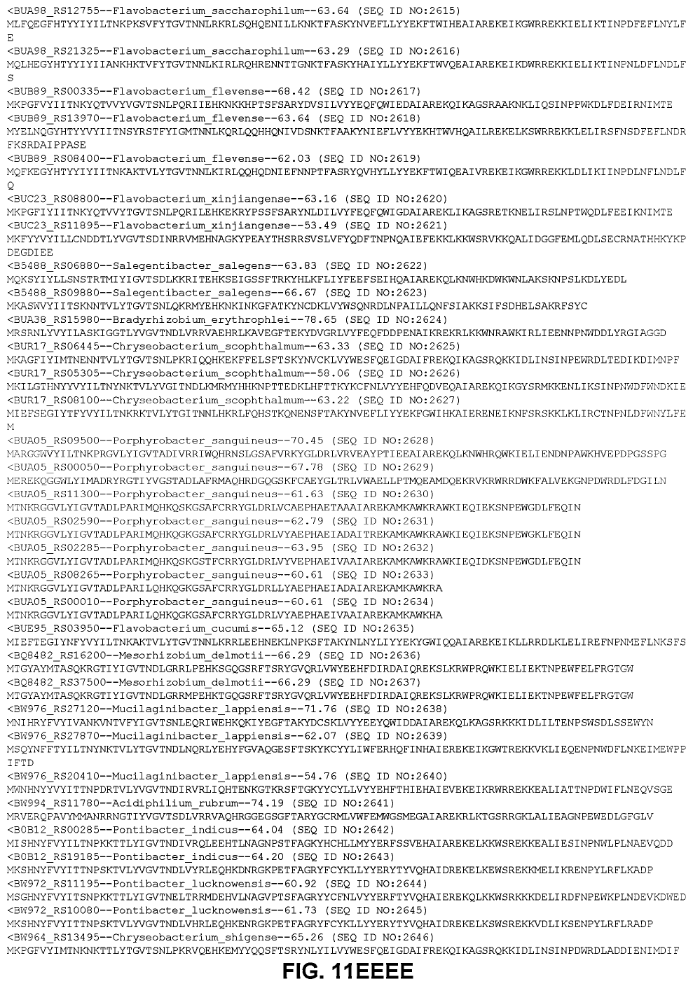

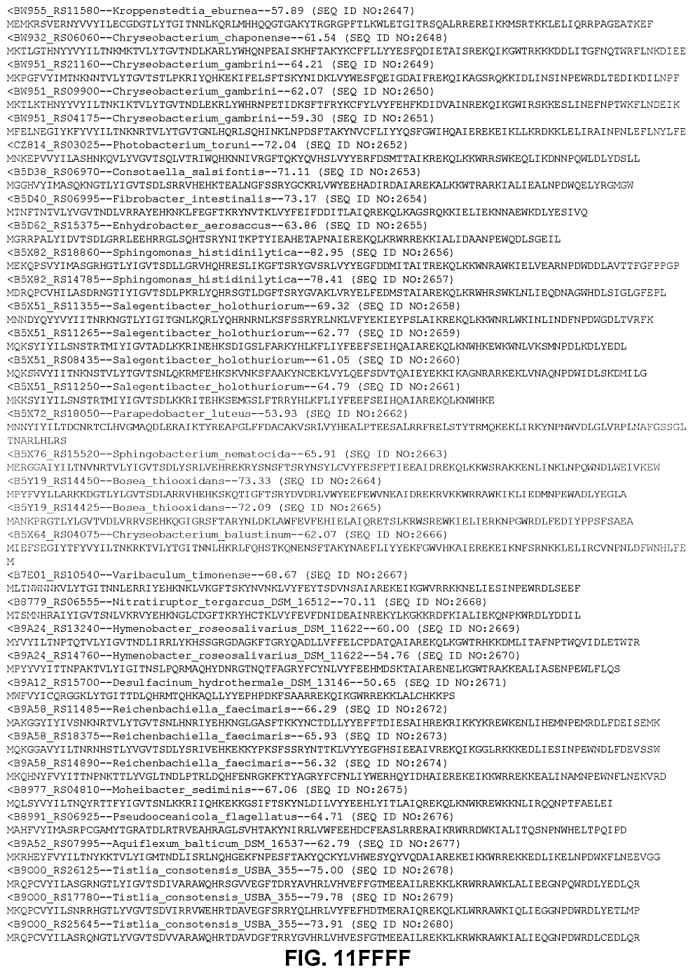

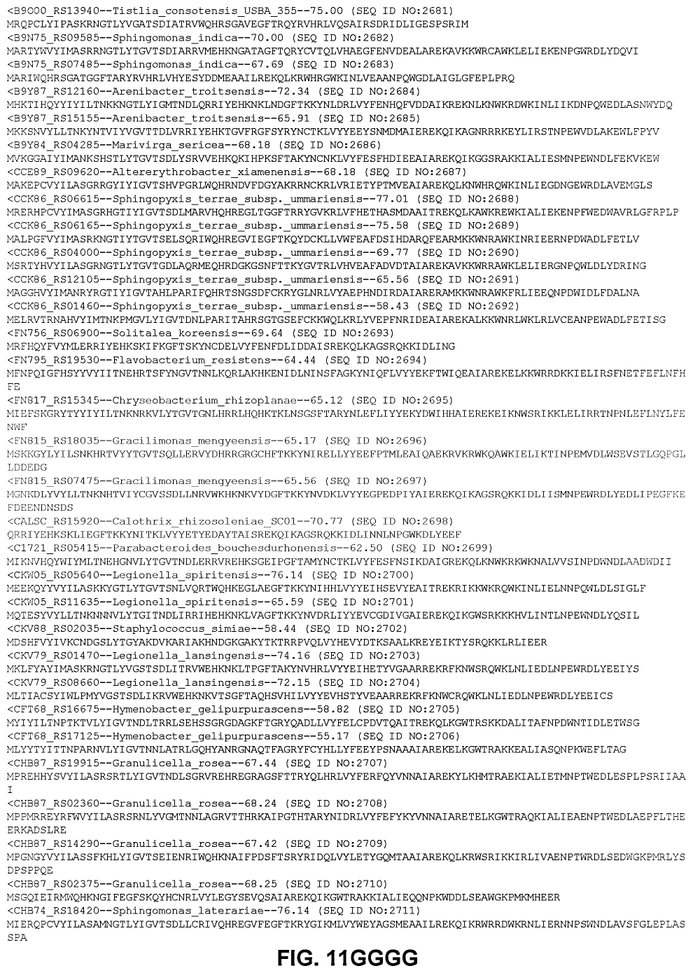

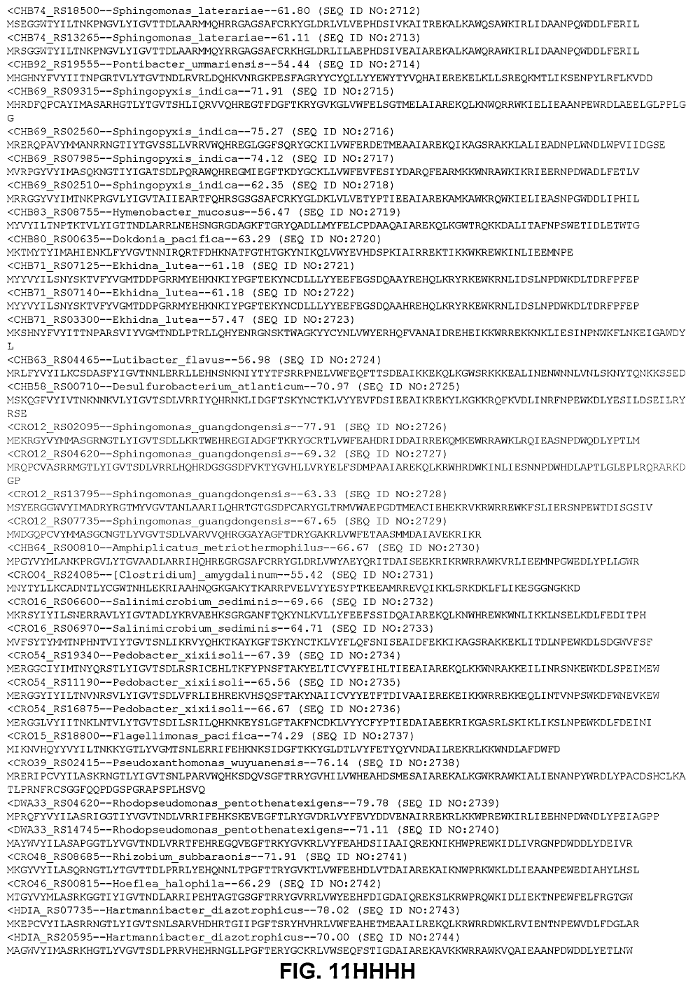

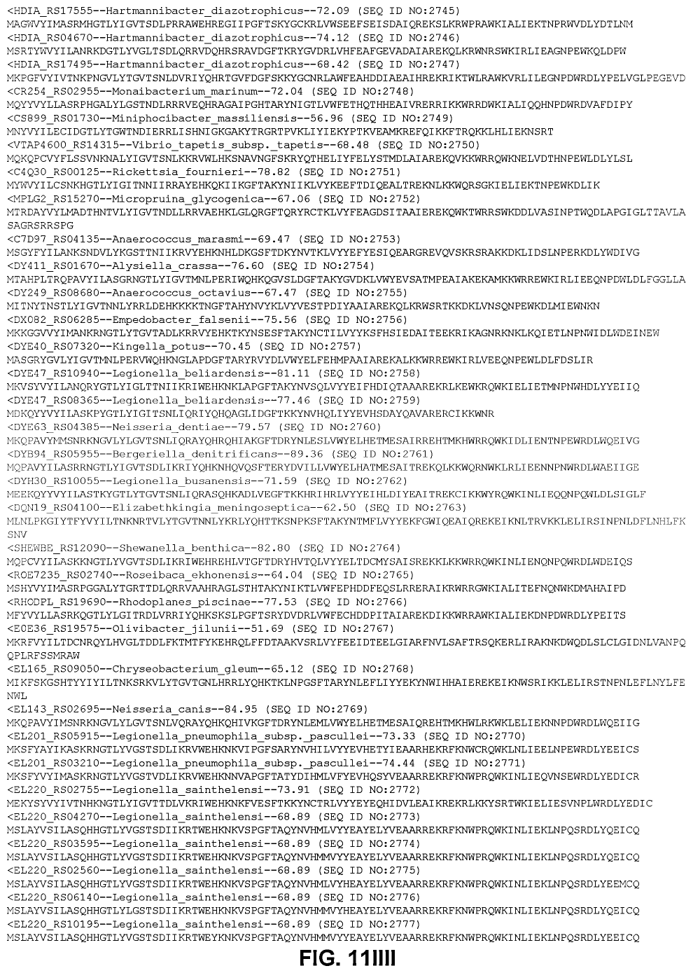

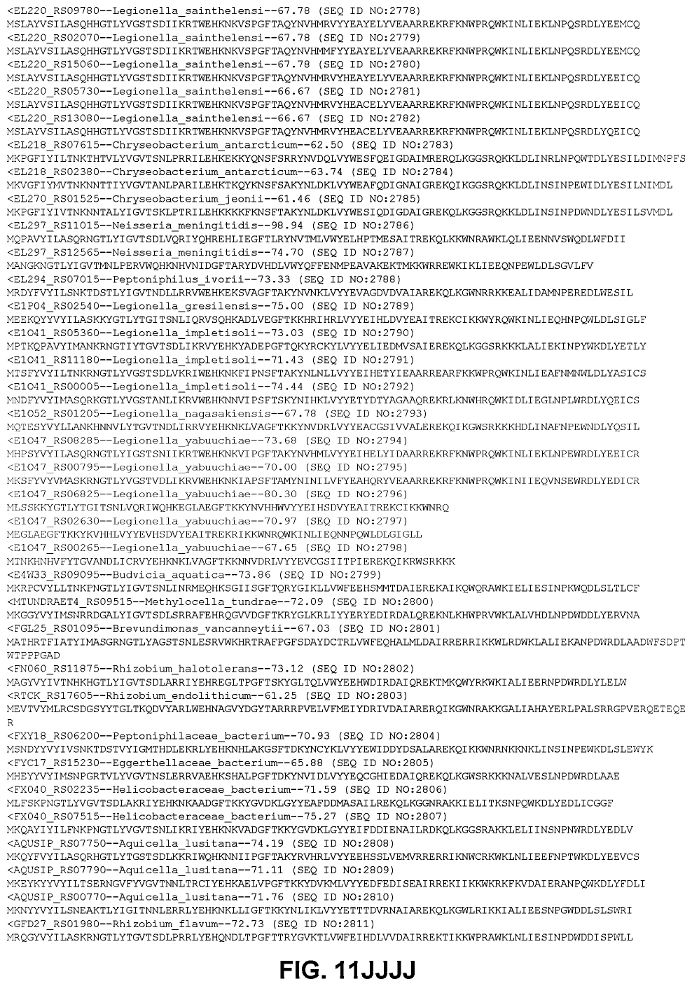

In an embodiment, the isolated endonuclease of the present disclosure comprises or consists of an amino acid sequence having at least 50% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891).

The term “endonuclease” as used herein refers to an enzyme having the ability to cleave a single-stranded nucleic acid molecule, such as single-stranded DNA, at or near a specific nucleotide sequence (recognition or restriction site).

The term “isolated” as used herein refers to a molecule (endonuclease) that is in a milieu or environment that is different from the natural milieu or environment where it is found in nature (i.e., that has been subjected to human manipulation), for example a endonuclease that has isolated from the natural bacteria that normally expressed it. As such, “isolated” does not necessarily reflect the extent to which the endonuclease has been purified, but indicates that the molecule has been separated in some way from the natural environment where it is normally found. An isolated endonuclease may also be produced recombinantly by cloning a nucleic acid encoding the endonuclease in a host cell capable of expressing the endonuclease, and collecting the endonuclease produced.

“Identity” refers to sequence identity between two polypeptides. Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as Clustal Omega, BLAST, BLAST-2, ALIGN or Megalign (DNASTAR) software. Appropriate parameters for aligning sequences are able to be determined, including algorithms needed to achieve maximal alignment over the full length of the sequences being compared.

Similarity refers to sequence similarity between two polypeptides. Percent (%) sequence similarity with respect to a reference polypeptide sequence is the percentage of amino acid residues in a candidate sequence that are similar (identical or conserved) with the amino acid residues in the reference polypeptide sequence, after aligning the sequences and introducing gaps, if necessary, to achieve the maximum percent sequence similarity, and considering conservative substitutions as part of the sequence similarity. The similarity between amino acids can be defined either by their chemical properties (e.g., hydrophobic, hydrophilic, charged, polar, etc.) or based on a PAM matrix.

Variations in the endonucleases described herein, can be made, for example, using any of the techniques and guidelines for conservative and non-conservative mutations set forth, for instance, in U.S. Pat. No. 5,364,934. Variations may be a substitution, deletion or insertion of one or more codons encoding the endonuclease that results in a change in the amino acid sequence as compared with the native sequence of the endonuclease. Optionally the variation is by substitution of at least one amino acid with any other amino acid in one or more of the domains of the endonuclease. Guidance in determining which amino acid residue may be inserted, substituted or deleted without adversely affecting the desired activity may be found by comparing the sequence of the endonuclease with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence (e.g., ability to cleave single-stranded nucleic acid molecules).

In an embodiment, the isolated endonuclease comprises or consists of an amino acid sequence having at least 55% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 60% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 65% similarity or identity with the any one of the sequences set forth in SEQ ID NOs: 2-5, FIGS. 11A-JJJJ (SEQ ID NO: 6-2811) and FIGS. 13A-B (SEQ ID NO: 2812-2891), preferably SEQ ID NOs: 2-5. In an embodiment, the isolated endonuclease comprises or consists of an amino acid sequence having at least 70% identity similarity or with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 75% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 80% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 85% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 90% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 95% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 96% similarity or identity with the any one of the sequences set forth in SEQ ID NOs: 2-5, FIGS. 11A-JJJJ (SEQ ID NO: 6-2811) and FIGS. 13A-B (SEQ ID NO: 2812-2891), preferably SEQ ID NOs: 2-5. In an embodiment, the isolated endonuclease comprises or consists of an amino acid sequence having at least 97% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 98% similarity or identity with the any one of the sequences set forth in SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of an amino acid sequence having at least 99% similarity or identity with the any one of the sequences set forth SEQ ID NOs: 2-5, FIGS. 11A-JJJJ (SEQ ID NO:6-2811) and FIGS. 13A-B (SEQ ID NO: 2812-2891), preferably SEQ ID NOs: 2-5. In an embodiment, the isolated endonuclease comprises or consists of or consists one of the amino acid sequences set forth in any one of SEQ ID NOs: 2-5, FIGS. 11A-JJJJ (SEQ ID NO:6-2811) and FIGS. 13A-B (SEQ ID NO: 2812-2891), preferably SEQ ID NOs: 2-5.

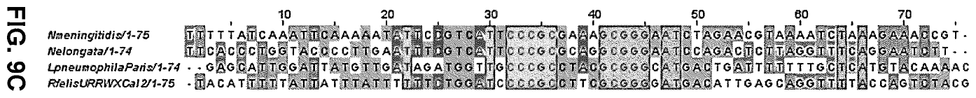

FIG. 9A depicts an alignment of the amino acid sequences of SEQ ID NOs: 2-5, with the residues conserved between the sequences indicated by asterisks (*), the residues with strong similarity (PAM250 MATRIX score between amino acids of greater than 0.5) indicated by colons (:), and the residues with weak similarity (PAM250 MATRIX score between amino acids of 0.5 or less) indicated by dots (.). In an embodiment, the isolated endonuclease comprises the conserved residues in SEQ ID NOs: 2-5. In an embodiment, the isolated endonuclease comprises the conserved residues and the residues with strong similarity in SEQ ID NOs: 2-5. In an embodiment, the isolated endonuclease comprises the conserved residues and the residues with strong and weak similarities in SEQ ID NOs: 2-5.

FIGS. 13A-B depict an alignment of the amino acid sequences of the GIY-YIG domains from 79 endonucleases belonging to the GIY-YIG_unchar_3 Conserved Protein Domain Family (CD10448), with the residues conserved between the sequences indicated by a #sign above the sequences. In an embodiment, the isolated endonuclease comprises the conserved residues in the sequences depicted in FIGS. 13A-B.

The isolated endonuclease may further comprise additional amino acids at its amino-(N) and/or carboxy (C)-terminal end. For example, the isolated endonuclease may be fused to a peptide or polypeptide, for example a peptide or polypeptide that may be used as an affinity tag to facilitate the detection and/or purification of the endonuclease. Examples of affinity tags include polyhistidine (His) tags, polyarginine tags, glutathione-S-transferase (GST) tags, FLAG tags, streptavidin-binding peptide or streptavidin-binding protein (SBP) tags, streptavidin-binding tag (Strep-tag), calmodulin-binding peptide (CBP) tags, chitin-binding tags, Maltose-binding protein (MBP) tags, and natural histidine affinity tags (HAT). The peptide or polypeptide may be fused directly to the N- and/or C-terminal end of the endonuclease, or indirectly via a linker. Such a linker may be a peptide/polypeptide linker comprising one or more amino acids or another type of chemical linker (e.g., a carbohydrate linker, a lipid linker, a fatty acid linker, a polyether linker, PEG, etc.) having suitable flexibility and stability to allow the endonuclease to adopt a proper conformation. The linker may comprise at least 2, 3 or 4 amino acids. The linker may comprise about 100, 90, 80, 70, 60 or 50 amino acids or less, and preferably 20, 15 or 10 amino acids or less.

The isolated endonuclease of the disclosure may be produced by expression in a host cell comprising a nucleic acid encoding the isolated endonuclease (recombinant expression) or by chemical synthesis (e.g., solid-phase peptide synthesis). Peptides and polypeptides can be readily synthesized by manual and automated solid phase procedures well known in the art. Suitable syntheses can be performed for example by utilizing “t-Boc” or “Fmoc” procedures. Techniques and procedures for solid phase synthesis are described in for example Solid Phase Peptide Synthesis: A Practical Approach, by E. Atherton and R. C. Sheppard, published by IRL, Oxford University Press, 1989. Alternatively, the polypeptides may be prepared by way of segment condensation, as described, for example, in Liu et al., Tetrahedron Lett. 37:933-936, 1996; Baca et al., J. Am. Chem. Soc. 117:1881-1887, 1995; Tarn et al., Int. J. Peptide Protein Res. 45:209-216, 1995; Schnolzer and Kent, Science 256:221-225, 1992; Liu and Tarn, J. Am. Chem. Soc. 116:4149-4153, 1994; Liu and Tarn, Proc. Natl. Acad. Sci. USA 91:6584-6588, 1994; and Yamashiro and Li, Int. J. Peptide Protein Res. 31:322-334, 1988). Other methods useful for synthesizing polypeptides are described in Nakagawa et al., J. Am. Chem. Soc. 107:7087-7092, 1985.

The isolated endonuclease may also be prepared using recombinant DNA technology using standard methods. Accordingly, in another aspect, the disclosure further provides a nucleic acid (e.g., mRNA, cDNA) encoding the above-mentioned endonuclease. The disclosure also provides a vector comprising the above-mentioned nucleic acid. In yet another aspect, the present disclosure provides a cell (e.g., a host cell) comprising the above-mentioned nucleic acid and/or vector. The disclosure further provides a recombinant expression system, vectors and host cells, such as those described above, for the expression/production of an endonuclease of the disclosure, using for example culture media, production, isolation and purification methods well known in the art.

The endonuclease of the disclosure can be purified by many techniques of peptide/polypeptide purification well known in the art, such as reverse phase chromatography, high performance liquid chromatography (HPLC), ion exchange chromatography, size exclusion chromatography, affinity chromatography, gel electrophoresis, and the like. The actual conditions used to purify a particular peptide or polypeptide will depend, in part, on synthesis strategy and on factors such as net charge, hydrophobicity, hydrophilicity, and the like, and will be apparent to those of ordinary skill in the art. For affinity chromatography purification, any ligand or antibody that specifically binds the endonuclease (or to an affinity tag fused to the endonuclease) may for example be used.

The present disclosure also provides a composition comprising (i) an isolated endonuclease specific for single-stranded nucleic acid molecules as described herein, and (ii) an aqueous saline solution or buffer.

The composition according to the present disclosure an aqueous saline solution or buffer. Such aqueous saline solutions or buffers include ingredients that stabilize the endonuclease and provide suitable conditions for the enzymatic activity of the endonuclease (e.g., conditions that permit the cleavage of single-stranded nucleic acids). The aqueous saline solution or buffer present in the composition according to the present disclosure may include suitable salts, buffering agents, minerals, co-factors, stabilizing agents, anti-oxidants, redox reagent, preservatives, etc.

In an embodiment, the composition comprises a buffering agent. The buffering agent is useful to keep the composition at a desired pH. Buffering agents are well known in the art and include potassium, acetate, citrate, acetate, phosphate, carbonate, succinate, histidine, borate, maleate, tris(hydroxymethyl) aminomethane (Tris), BIS-Tris, piperazine-N,N′-bis(2-ethanesulfonic acid) (PIPES), 2-(N-morpholino) ethanesulfonic acid (MES), (3-(N-morpholino) propanesulfonic acid) (MOPS), N-(2-Acetamido)-2-aminoethanesulfonic acid (ACES), (4-(2-hydroxyethyl)-1-piperazineethanesulfonic acid) (HEPES), magnesium and hydrochloride buffers. In an embodiment, the composition comprises a Tris buffer. In a further embodiment the Tris buffer is a Tris-HCl or a Tris-acetate buffer. In an embodiment, the buffering agent is at a concentration of about 0.1 mM to 1M. In further embodiments, the buffering agent is at a concentration of about 1 mM to about 500 mM, about 1 mM to about 200 mM, about 1 mM to about 100 mM, about 5 mM to about 100 mM, about 5 mM to about 75 mM, about 5 mM to about 50 mM, or about 5 mM to about 25 or 20 mM. In an embodiment, the buffering agent is at a concentration of about 10 mM. In an embodiment, the buffering agent has a pH of about 5 to about 10. In further embodiments, the buffering agent has a pH of about 6 to about 9, of about 6.5 to about 9, of about 7 to about 9, of about 7.5 to about 8.5, or of about 7.6 to about 8.2. In an embodiment, the buffering agent has a pH of about 7.9 or 8.0. In an embodiment, the pH is the pH at a temperature of about 20 to about 40° C. In an embodiment, the pH is the pH at a temperature of about 20 or 25° C. In an embodiment, the pH is the pH at a temperature of about 37° C.

In an embodiment, the composition comprises a salt, such as a metal salt. Common salt-forming cations include ammonium (NH₄⁺), manganese, nickel, calcium, iron, magnesium, potassium, sodium and copper. In an embodiment, the metal salt is a magnesium salt, manganese salt or zinc salt. Common salt-forming anions include acetate, carbonate, chloride, citrate, fluoride, nitrate, nitrite, oxide, phosphate and sulfate. Examples of salts include magnesium chloride (MgCl₂), magnesium acetate, potassium acetate (KCH₃CO₂), potassium chloride (KCl), sodium acetate (CH₃COONa), sodium chloride (NaCl), calcium chloride, zinc chloride, manganese sulfate, manganese chloride, nickel chloride, nickel acetate, and sodium sulfate (Na₂SO₄). In an embodiment, the salt comprises a magnesium, manganese, nickel and/or sodium cation. In an embodiment, the salt comprises a chloride anion. In an embodiment, the composition comprises KCl. In an embodiment, the composition comprises KCl and NaCl. In an embodiment, the concentration of salt in the composition is about 1 mM to about 500 mM. In further embodiments, the concentration of salt in the composition is about 10 mM to about 300 mM, about 20 mM to about 200 mM, about 20 mM to about 150 mM, about 30 mM to about 150 mM. In an embodiment, the composition comprises a salt comprising a magnesium cation (e.g., MgCl₂) at a concentration of about 1 mM to about 100 mM, for example about 1 mM to about 50 mM, about 5 mM to about 20 mM, or about 5 to about 15 mM. In an embodiment, the composition comprises a salt comprising a magnesium cation (e.g., MgCl₂) at a concentration of about 10 mM. In an embodiment, the composition comprises a salt comprising a sodium cation (e.g., NaCl) at a concentration of about 1 mM to about 200 mM, about 10 mM to about 150 mM, about 10 mM to about 100 mM, about 25 mM to about 75 mM. In an embodiment, the composition comprises a salt comprising a sodium cation (e.g., NaCl) at a concentration of about 50 mM.

In an embodiment, the composition comprises a stabilizing agent, such as a protein. In an embodiment, the protein is albumin, such as bovine serum albumin (BSA). In an embodiment, the stabilizing agent is at a concentration of about 1 μg/ml to about 1 mg/ml. In an embodiment, the stabilizing agent is at a concentration of about 10 μg/ml to about 500 μg/ml. In an embodiment, the stabilizing agent is at a concentration of about 50 μg/ml to about 200 μg/ml. In an embodiment, the stabilizing agent is at a concentration of about 50 μg/ml to about 150 μg/ml. In an embodiment, the stabilizing agent is at a concentration of about 80 to about 120 μg/ml, for example about 100 μg/ml.

In embodiments, the composition further comprises additional ingredients, such as detergents (e.g., non-ionic detergents like Triton® X-100 or Tween® 20) and/or redox reagents (DTT, beta-mercaptoethanol). For storage, the composition may further comprise a metal-chelating agent, such as ethylenediaminetetraacetic acid (EDTA).

Aqueous solutions/buffers for endonucleases are available commercially from several providers such as New England Biolabs Inc., Pomega and Thermo Scientific. Examples from New England Biolabs Inc. include NEBuffer 1 (10 mM Bis-Tris-Propane-HCl, 10 mM MgCl₂, 1 mM DTT, pH 7.0@25° C.); NEBuffer 1.1 (10 mM Bis-Tris-Propane-HCl, 10 mM MgCl₂, 100 μg/ml BSA. pH 7.0@25° C.); NEBuffer 2.1 (50 mM NaCl, 10 mM Tris-HCl, 10 mM MgCl₂, 100 μg/ml BSA, pH 7.9@25° C.); NEBuffer 3.1 (100 mM NaCl, 50 mM Tris-HCl. 10 mM MgCl₂, 100 μg/ml BSA, pH 7.9@25° C.); NEBuffer 4 (50 mM Potassium acetate, 20 mM Tris-acetate, 10 mM Magnesium Acetate, 1 mM DTT, pH 7.9@25° C.); and CutSmart Buffer (50 mM Potassium Acetate, 20 mM Tris-acetate. 10 mM Magnesium Acetate, 100 μg/ml BSA, pH 7.9@25° C.). Examples from Thermo Scientific include Buffer B (10 mM Tris-HCl (pH 7.5 at 37° C.), 10 mM MgCl₂, 0.1 mg/ml BSA), Buffer G (10 mM Tris-HCl (pH 7.5 at 37° C.), 10 mM MgCl₂, 50 mM NaCl, 0.1 mg/ml BSA), Buffer O (50 mM Tris-HCl (pH 7.5 at 37° C.), 10 mM MgCl₂, 100 mM NaCl, 0.1 mg/ml BSA), Buffer R (10 mM Tris-HCl (pH 8.5 at 37° C.), 10 mM MgCl₂, 100 mM KCl, 0.1 mg/ml BSA), and Buffer Tango™ (33 mM Tris-acetate (pH 7.9 at 37° C.), 10 mM magnesium acetate, 66 mM potassium acetate, 0.1 mg/ml BSA). Examples from Promega include Buffer A (6 mM Tris-HCl (pH 7.5 at 37° C.), 6 mM MgCl₂, 6 mM NaCl, 1 mM DTT), Buffer B (6 mM Tris-HCl (pH 7.5 at 37° C.), 6 mM MgCl₂, 50 mM NaCl, 1 mM DTT), Buffer C (10 mM Tris-HCl (pH 7.9 at 37° C.), 10 mM MgCl₂, 50 mM NaCl, 1 mM DTT), Buffer D (6 mM Tris-HCl (pH 7.9 at 37° C.), 6 mM MgCl₂, 150 mM NaCl, 1 mM DTT), Buffer E (6 mM Tris-HCl (pH 7.5 at 37° C.), 6 mM MgCl₂, 100 mM NaCl, 1 mM DTT), Buffer F (10 mM Tris-HCl (pH 8.5 at 37° C.), 10 mM MgCl₂, 100 mM NaCl, 1 mM DTT), Buffer G (50 mM Tris-HCl (pH 8.2 at 37° C.), 5 mM MgCl₂), Buffer H (90 mM Tris-HCl (pH 7.5 at 37° C.), 10 mM MgCl₂, 50 mM NaCl), Buffer J (10 mM Tris-HCl (pH 7.5 at 37° C.), 7 mM MgCl₂, 50 mM KCl, 1 mM DTT), Buffer K (10 mM Tris-HCl (pH 7.4 at 37° C.), 10 mM MgCl₂, 150 mM KCl), Buffer L (10 mM Tris-HCl (pH 9.0 at 37° C.), 3 mM MgCl₂, 100 mM NaCl), and MULTI-CORE™ Buffer (25 mM Tris-Acetate, pH 7.5 (at 37° C.), 100 mM potassium acetate, 10 mM magnesium acetate, 1 mM DTT).

In an embodiment, the composition comprises an aqueous solution/buffer comprising:
from about 1 to about 100 mM of a buffering agent, such as a Tris-based buffering agent (e.g., Tris-HCl), having a pH of about 7 to about 9;
from about 1 to about 100 mM of a metal salt, such as a salt comprising a magnesium and/or sodium cation (NaCl and/or KCl); and
from about 10 μg/ml to about 500 μg/ml of a stabilizing agent, such as a protein (e.g., albumin).

In a further embodiment, the composition comprises an aqueous solution/buffer comprising:
from about 5 to about 20 mM of a buffering agent, such as a Tris-based buffering agent (e.g., Tris-HCl), having a pH of about 7.5 to about 8.5;
from about 10 to about 100 mM of a metal salt, such as a metal salt comprising a magnesium and/or sodium cation (NaCl and/or KCl); and
from about 50 μg/ml to about 200 μg/ml of a stabilizing agent, such as a protein (e.g., albumin).

In a further embodiment, the composition comprises an aqueous solution/buffer comprising:
about 10 mM of a buffering agent, such as a Tris-based buffering agent (e.g., Tris-HCl), having a pH of about 7.9 or 8.0;
about 50 mM of NaCl; and
about 100 μg/ml of albumin (e.g., BSA).

In a further embodiment, the composition comprises an aqueous solution/buffer comprising:
about 10 mM of a buffering agent, such as a Tris-based buffering agent (e.g., Tris-HCl), having a pH of about 7.9 or 8.0;
about 50 mM of NaCl;
about 10 mM of MgCl₂; and
about 100 μg/ml of BSA.

The present disclosure also provides a mixture comprising the above-described isolated endonuclease or composition and a single-stranded nucleic acid molecule (e.g., single-stranded DNA).

The present disclosure also provides a method for cleaving a single-stranded nucleic acid molecule comprising contacting the single-stranded nucleic acid molecule with the isolated endonuclease or composition defined herein under conditions suitable for cleavage of the single-stranded nucleic acid molecule by the isolated endonuclease.

The results presented in the Examples below show that the endonucleases according to the present disclosure recognize specific nucleotide sequences within the single-stranded nucleic acid molecules. The recognition motif contains a ≈30 nt repeated sequence (NTS) that is predicted to form a stem-loop secondary structure. The cleavage of the single-stranded nucleic acid molecules occurs just outside the NTS.

In an embodiment, the single-stranded nucleic acid molecule cleaved by the endonuclease comprises a nucleotide sequence having at least 50% sequence identity with the sequence:

(SEQ ID NO: 2917)
GTCATTCCCnnnnnnnnGGGAATC
or
(SEQ ID NO: 2918)
GUCAUUCCCnnnnnnnnGGGAAUC.

In an embodiment, the single-stranded nucleic acid molecule cleaved by the endonuclease comprises a nucleotide sequence having at least 50% sequence identity with the sequence:

(SEQ ID NO: 2919)
GTCATTCCCGCGAAAGCGGGAATC
or
(SEQ ID NO: 2920)
GUCAUUCCCGCGAAAGCGGGAAUC.

In an embodiment, the single-stranded nucleic acid molecule comprises the following nucleotide sequence:

(SEQ ID NO: 2921)
GTCANNCCNGNNNANNCNGGNNNC
or
(SEQ ID NO: 2922)
GUCANNCCNGNNNANNCNGGNNNC.

The identification of nucleotides in the present disclosure is according to the standard 1-letter nomenclature from the International Union of Pure and Applied Chemistry (IUPAC):

| IUPAC 1-letter code | Base |
|---|---|
| A | Adenine |
| C | Cytosine |
| G | Guanine |
| T (or U) | Thymine (or Uracil) |
| R | A or G |
| Y | C or T/U |
| S | G or C |
| W | A or T/U |
| K | G or T/U |
| M | A or C |
| B | C or G or T/U |
| D | A or G or T/U |
| H | A or C or T/U |
| V | A or C or G |
| N | any base |

In an embodiment, the single-stranded nucleic acid molecule comprises the following nucleotide sequence:

(SEQ ID NO: 2923)
GTCAYBCCMGYRHAVRCKGGVRNC
or
(SEQ ID NO: 2924)
GUCAYBCCMGYRHAVRCKGGVRNC.

In an embodiment, the single-stranded nucleic acid molecule comprises a sequence having at least 50, 60, 70, 80, 90, 95 or 100% identity with one of the following nucleotide sequences:

(SEQ ID NO: 2925)
GTCATCCCCGCGCAGGCGGGGACCC
or
(SEQ ID NO: 2926)
GUCAUCCCCGCGGCAGGCGGGACCC;
(SEQ ID NO: 2927)
GTCATTCCCGCGCAGGCGGGAATCC
or
(SEQ ID NO: 2928)
GUCAUUCCCGCGCAGGCGGGAAUCC;
(SEQ ID NO: 2929)
GTCATTCCCGCGAAAGCGGGAATCC
or
(SEQ ID NO: 2930)
GUCAUUCCCGCGAAAGCGGGAAUCC;
(SEQ ID NO: 2931)
GTCATTCCCGCGAAGGCGGGAATCC
or
(SEQ ID NO: 2932)
GUCAUUCCCGCGAAGGCGGGAAUCC;
(SEQ ID NO: 2933)
GTCATTCCCGCGCAGGCGGGAATCC
or
(SEQ ID NO: 2934)
GUCAUUCCCGCGCAGGCGGGAAUCC;
(SEQ ID NO: 2935)
GTCATCCCCGCGCAGGCGGGGACCC
or
(SEQ ID NO: 2936)
GUCAUCCCCGCGCAGGCGGGGACCC;
(SEQ ID NO: 2937)
GTCATGCCCGCGTAGGCGGGCAACC
or
(SEQ ID NO: 2938)
GUCAUGCCCGCGUAGGCGGGCAACC;
(SEQ ID NO: 2939)
GTCATTCCCGCGAAAGCGGGAAGCC
or
(SEQ ID NO: 2940)
GUCAUUCCCGCGAAAGCGGGAAGCC;
(SEQ ID NO: 2941)
GTCATTCCCGCGCAGGCGGGAATCC
or
(SEQ ID NO: 2942)
GUCAUUCCCGCGCAGGCGGGAAUCC;
(SEQ ID NO: 2943)
GTCATTCCCGTGCACACGGGAATCC
or
(SEQ ID NO: 2944)
GUCAUUCCCGUGCACACGGGAAUCC;
(SEQ ID NO: 2945)
GTCATGCCCGCGCAGGCGGGCATCC
or
(SEQ ID NO: 2946)
GUCAUGCCCGCGCAGGCGGGCAUCC;
(SEQ ID NO: 2947)
GTCATCCCCGCGAAGGCGGGGATCC
or
(SEQ ID NO: 2948)
GUCAUCCCCGCGAAGGCGGGGAUCC;
(SEQ ID NO: 2949)
GTCATTCCCGCGAAAGCGGGAATCC
or
(SEQ ID NO: 2950)
GUCAUUCCCGCGAAAGCGGGAAUCC;
(SEQ ID NO: 2951)
GTCATTCCCGCGAAGGCGGGAATCC
or
(SEQ ID NO: 2952)
GUCAUUCCCGCGAAGGCGGGAAUCC;
(SEQ ID NO: 2953)
GTCATTCCCGCGCAGGCGGGAATCT
or
(SEQ ID NO: 2954)
GUCAUUCCCGCGCAGGCGGGAAUCU;
(SEQ ID NO: 2955)
GTCATTCCCGCGTAGGCGGGAATCC
or
(SEQ ID NO: 2956)
GUCAUUCCCGCGUAGGCGGGAAUCC;
(SEQ ID NO: 2957)
GTCACTCCCGCGAAGGCGGGAGTCC
or
(SEQ ID NO: 2958)
GUCACUCCCGCGAAGGCGGGAGUCC;
(SEQ ID NO: 2959)
GTCACCCCAGCGAAAGCTGGGGTCC
or
(SEQ ID NO: 2960)
GUCACCCCAGCGAAAGCUGGGGUCC;
(SEQ ID NO: 2961)
GTCATTCCCGCACAGGCGGGAATCC
or
(SEQ ID NO: 2962)
GUCAUUCCCGCACAGGCGGGAAUCC;
and
(SEQ ID NO: 2963)
GTCATTCCCGCGCAGGCGGGAATCT
or
(SEQ ID NO: 2964)
GUCAUUCCCGCGCAGGCGGGAAUCU.

As shown in the examples below, the genes encoding the endonucleases according to the present disclosure are typically surrounded by one or several repeats of their own recognition sequences in the genome of the bacteria. Thus, the skilled person would be able to easily identify the recognition sequence of any endonuclease according to the present disclosure by identifying repeating nucleotide sequences/motifs located near (i.e., just before/upstream and/or after/downstream) the gene encoding the endonuclease. Such recognition sequence is expected to have some level of sequence identity with the recognition sequences disclosed herein.

The method according to the present disclosure comprises incubating the single-stranded nucleic acid molecule with the composition defined herein for a period of time and under conditions suitable for cleavage of the single-stranded nucleic acid molecule by the endonuclease.

In an embodiment, the period of time is at least 5 minutes. In further embodiments, the period of time is at least 10 or 15 minutes. In yet further embodiments, the period of time is at least 20, 30 or 45 minutes. In embodiments, the period of time is from about 15 minutes to about 2 hours, from about 20 minutes to about 90 minutes, from about 30 minutes to about 60 minutes, or from about 45 to about 60 minutes.

In an embodiment, the conditions for incubation comprise a temperature of about 10, 15 or 20° C. to about 60, 55 or 50° C. In further embodiments, the conditions for incubation comprise a temperature of about 25 or 30° C. to about 40 or 45° C., such as a temperature of about 35 to about 40° C., e.g., about 36, 37 or 38° C.

In an embodiment, the conditions for incubation comprise the presence of a suitable amount of a metal, such as a divalent metal. Examples of divalent metals include magnesium, manganese, cadmium, calcium, cobalt, nickel, zinc, iron and copper. In a further embodiment, the divalent metal is magnesium, manganese or nickel, preferably magnesium. If the divalent metal is not present in the initial composition comprising the endonuclease, a suitable amount of the divalent metal is added to the reaction mixture prior to and/or during the incubation period. The divalent metal may be in the form of a salt, such as the salts listed above. In an embodiment, the divalent metal is magnesium and is in the form of magnesium chloride (MgCl₂). In an embodiment, the concentration of metal salt (e.g., MgCl₂) present during the incubation period is about 1 mM to about 100 mM, for example about 1 mM to about 50 mM, about 5 mM to about 20 mM, about 5 to about 15 mM, or about 10 mM.

In an embodiment, the conditions for incubation comprise a pH of about 5 to about 10. In further embodiments, the conditions for incubation comprise a pH of about 6 to about 9, of about 6.5 to about 9, of about 7 to about 9, of about 7.5 to about 8.5, or of about 7.6 to about 8.2. In an embodiment, the conditions for incubation comprise a pH of about 7.9 or 8.0.

The amount of the endonuclease relative to that of the single-stranded nucleic acid molecule may be adjusted to obtain a suitable cleavage efficiency. In an embodiment, the [concentration of endonuclease]/[concentration of single-stranded nucleic acid molecule] ratio is at least 0.00005, 0.0001, 0.001, 0.01, 0.05, 0.1 or 0.5. In an embodiment, the [concentration of endonuclease]/[concentration of single-stranded nucleic acid molecule] ratio is from 0.01 to 100, from 0.05 to 50 or from 0.1 to 10.

In another aspect, the present disclosure provides a method for rendering a single-stranded nucleic acid susceptible to cleavage by the endonuclease described herein, the method comprising incorporating the nucleotide sequence defined above (recognition motif) into the single-stranded nucleic acid. The incorporation of the nucleotide sequence (recognition motif) may be achieved by adding the nucleotide sequence defined above (or a portion thereof) at the 5′-end, 3′-end or within the single-stranded nucleic acid, and/or by introducing one or more mutations (e.g., substitutions) within the sequence of the single-stranded nucleic acid to obtain the desired nucleotide sequence (recognition motif). Methods to modify nucleic acids are well known in the art and include, for example, cassette mutagenesis, PCR site-directed mutagenesis and genome-editing technologies using nucleases such as zinc finger nucleases (ZPNs) (Gommans et al., J. Mol Biol, 354 (3): 507-519 (2005)), transcription activator-like effector nucleases (TALENs) (Zhang et al., Nature Biotechnol, 29:149-153 (2011)), the CRISPR/Cas system (Cheng et al., Cell Res., 23:1163-71] (2013)), and engineered meganucleases (Riviere et al., Gene Ther., 21 (5): 529-32 (2014)).

The present disclosure also provides a method for expressing the endonuclease defined herein in a cell, the method comprising introducing a nucleic acid encoding the endonuclease into the cell. The cell may be a procaryotic or eucaryotic cell. The nucleic acid may be an mRNA or cDNA molecule, naked or incorporated into a vector or plasmid, and it may be incorporated into the cell using any suitable methods for introducing nucleic acids into a cell (e.g., transfection, transformation, etc.).

In an embodiment, the cell comprises a single-stranded nucleic acid that is cleaved by the endonuclease.

In another aspect, the present disclosure provides a kit or commercial package comprising the endonuclease or composition described herein. In an embodiment, the kit or package further comprises instructions setting forth a method for cleaving a single-stranded nucleic acid with the endonuclease, such as the method described herein. The kit or package may further comprise various components such as solutions or buffers (e.g., a reaction buffer) such as those described herein, containers, vials, etc.

### EXAMPLES

The present disclosure is illustrated in further details by the following non-limiting examples.

### Example 1: Materials and Methods

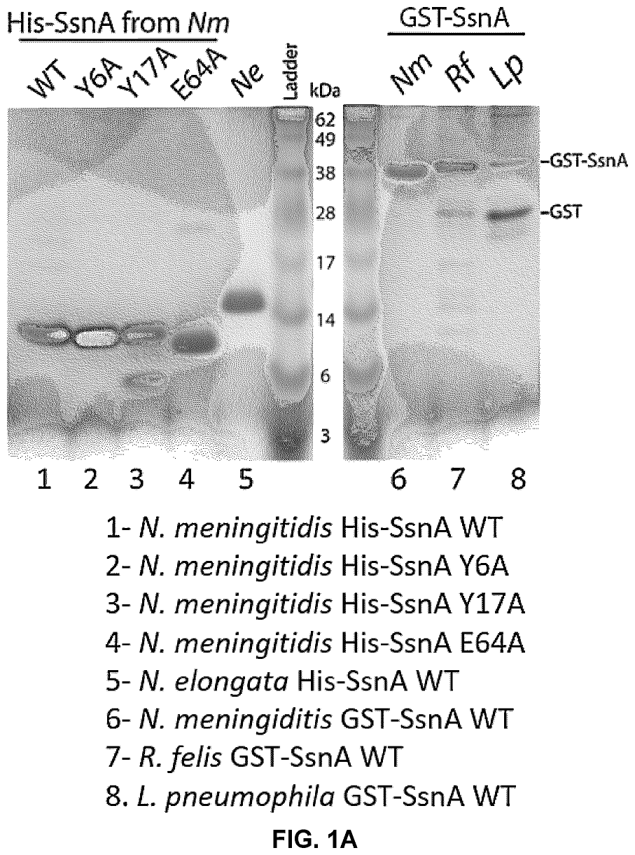

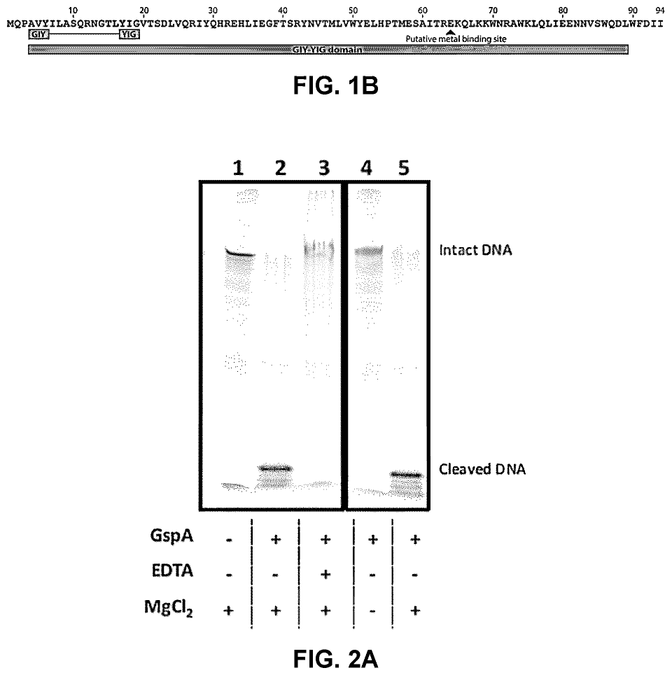

Gene expression and purification (FIG. 1A). The NMV0044 gene was amplified from N. meningitidis 8013 2C4.3 by Phusion PCR using primers containing the NdeI and XhoI restriction sites, then cloned into pET15-MHL expression vector, generating a recombinant GIY-YIG small protein A (SsnA) with a 6×His-tag in N-terminal. The E64A, Y6A and Y17A mutants were also generated using site-directed mutagenesis by PCR in conserved residues expected to be critical for enzymatic activity (FIG. 1B). Expression was induced at exponential phase with 1 mM IPTG for 4 hours at 37° C., followed by overnight incubation at 23° C. Cells were pelleted, lyzed by lysozyme treatment and sonication, and the soluble fraction was purified by affinity chromatography with Ni-NTA resin (Thermo). >95% pure SsnA was obtained with 300 mM imidazole elution buffer (FIG. 1A). The protein was stored at −80° C.

SsnA(NMV0044) gene Nm2C4.3
(SEQ ID NO: 1)
atgcagcctgcggtttatattttagcaagccaacgtaatggcacgttat
acattggcgttacatctgatttggtgcaacgtatttaccaacataggga
acatttgattgagggatttacatcacggtacaacgttactatgctggtt
tggtatgaactgcatcctacgatggagagcgcaattactcgggaaaaac
agttgaagaaatggaacagggcttggaaattgcaactgattgaagaaaa
taatgtttcttggcaggatttatggtttgatattatttag

GeneID: 12395323 Altname: NMV_RS00225 Accession_number: WP_002216166.1 Prot_locus: CAX49033. Embl_accession: FM999788.1. The SsnA gene is known as NMB0047 or NMB_RS00250 (CDS WP_002216166.1) in the reference strain N. meningitidis MC58, and NMA0292 or NMA_RS01540 in N. meningitidis Z2491 (CDS WP_002246543.1).

SsnA(NMV0044) protein Nm2C4.3
(SEQ ID NO: 2)
MQPAVYILASQRNGTLYIGVTSDLVQRIYQHREHLIEGFTSRYNVTMLV
WYELHPTMESAITREKQLKKWNRAWKLQLIEENNVSWQDLWFDII

For SsnA homologs from different species, NEIELOOT_01219 (EFE49965.1) was taken from Neisseria elongata subsp. glycolytica, WP_011213498 was taken from Legionella pneumophila subsp. pneumophila str Paris and WP_011271370 was taken from Rickettria felis URRWXCal2. Expression and purification of 6×His-recombinant proteins were done by affinity chromatography using a nickel resin. Expression and purification of GST-recombinant proteins were done by affinity chromatography using a glutathione resin (FIG. 1A).

SsnA(EFE49965.1) N.elongata
(SEQ ID NO: 3)
MQPAVYILASQRNGTLYIGVTSNLTQRVYQHREHLVQGFTNQHHVTLLV
WYELHSTMEHAITREKQLKKWNRQWKLRLIEEKNPSWQDLWFEIIK
SsnA(WP_011213498) L. pneumophila Paris
(SEQ ID NO: 4)
MEEKQYWYILASKAYGTLYTGITSNLVQRIYQHKKGLAEGFTKRYNVHR
LVYYEIHTDVYEAITREKRIKKWNRQWKINLIEQKNPQWLDLSIGLC
SsnA(WP_011271370) R.felis URRWXCal2
(SEQ ID NO: 5)
MYWVYILCSDRNGTLYIGITNNILRRTYEHKQKIIKGFTAKYNIIKLVY
TEEFTDIKEALAREKALKKWNRAWKIKLIEKINLRWEDLGKCISGFPPT
RE

Electrophoretic mobility shift assay (EMSA). Gel shift assays, or EMSA, were performed by diluting the proteins in Diluent A (NEB) to the indicated working concentrations. 5′carboxyfluorescin-tagged oligos corresponding to genomic regions of N. meningitidis MC58 were synthesized from Sigma. When needed, complementary oligonucleotides were annealed by mixing equimolar amounts in annealing buffer (10 mM Tris-HCl PH8, 50 mM NaCl, 1 mM EDTA), incubating them 5 minutes at 95° C. and letting them slowly cool down. For the gel-shift assays, proteins were mixed with the fluorescent oligonucleotides in a reaction buffer containing 50 mM NaCl, 10 mM Tris-HCl, 100 μg/ml BSA, pH7.9. The mixes were incubated at 37° C. for 30 minutes before adding native loading dye. Samples were resolved on native 10% TBE-acrylamide gels and imaged with a Typhoon FLA9500 scanner. For branched DNA binding assays, a similar approach was used but the gel was stained with GelStain (Biotium) and imaged on a GelDoc (BioRad) since the oligonucleotides were not fluorescently labelled.

Nuclease assays. Nuclease assays were performed similarly to gel-shift assays, with the addition of 10 mM MgCl₂ in the reaction buffer. Reactions were stopped by adding formamide loading dye and incubating 3 minutes at 95° C. Samples were resolved on denaturing 17.5% TBE-Urea (8M) acrylamide gels, and imaged with a Typhoon FLA9500 scanner. The sequences of the DNA constructs used in the studies described herein are depicted in the table below.

| Name | Sequence |
|---|---|
| SSDNA | aattcaaaaatattccgtcattcccgcgaaagcgggaatctagaacgtaaaatctaaagaaaccgttttatccga |
| | taagtttccgcaccgacagacctgg (SEQ ID NO: 2965) |
| ssDNArev | ccaggtctgtcggtgcggaaacttatcggataaaacggtttctttagattttacgttctagattcccgctttcgcggg |
| | aatgacggaatatttttgaatt (SEQ ID NO: 2966) |
| dsDNA | aattcaaaaatattccgtcattcccgcgaaagcgggaatctagaacgtaaaatctaaagaaaccgttttatccga |
| | taagtttccgcaccgacagacctgg (SEQ ID NO: 2965) |
| SSDNA75 | tttttatcaaattcaaaaatattccgtcattcccgcgaaagcgggaatctagaacgtaaaatctaaagaaaccgt |
| | (SEQ ID NO: 2967) |
| SSDNA37 | aaaaatattccgtcattcccgcgaaagcgggaatcta (SEQ ID NO: 2968) |
| RNA37 | aaaaauauuccgucauucccgcgaaagcgggaaucua (SEQ ID NO: 2969) |
| SSDNA28 | ccgtcattcccgcgaaagcgggaatcta (SEQ ID NO: 2970) |
| SSDNA75 | uuuuuaucaaauucaaaaauauuccgucauucccgcgaaagcgggaaucuagaacquaaaaucua |
| (T→U) | aagaaaccgu (SEQ ID NO: 2971) |
| NTS#1 | gaggtttcgggcaacttctaaaccgtcattcccgcgcaggcgggaatccagaccattggacagcagcaatattc |
| | aaagattatctgaaagtccgggattc (SEQ ID NO: 2972) |
| NTS#2 | gtcattcccacgaacctacattccgtcattcccacgaaagtgggaatctagaatctctaaagcttcagctaaccttt |
| | gaatattgctgttgttctaaggt (SEQ ID NO: 2973) |
| NTS#3 | atcccgtcattcccgccactttccgtcattcccgcgaaagcgggaatctagaatctcggactttcagataatctttga |
| | atattgctgttgttctaaggtc (SEQ ID NO: 2974) |
| NTS#4 | taaatgaccccaacttaaaatcccgtcattcccgcgcaggcgggaatctaggtctgtcggtgcggaaacttatcg |
| | ggtaaaacggtttcttgagattttg (SEQ ID NO: 2975) |
| NTS#5 | gtcattcccacgaacctacattccgtcattcccacgaaagtgggaatccagtgcgttgagtttcagtcatttccaat |
| | aaattgccttagtattgaatgtc (SEQ ID NO: 2976) |
| Holliday_ | 1-Aggaacctcgagggatccgtcctagcaagccgctgctaccggaagcttctcgaggttcct (SEQ ID |
| junc | NO: 2977) |
| | 2-Aggaacctcgagaagcttccggtagcagcgagagcggtggttgaattcctcgaggttcct (SEQ ID |
| | NO: 2978) |
| | 3-Aggaacctcgaggaattcaaccaccgctcttctcaactgcagtctagactcgaggttcct (SEQ ID |
| | NO: 2979) |
| | 4-Aggaacctcgagtctagactgcagttgagagcttgctaggacggatccctcgaggttcct (SEQ ID |
| | NO: 2980) |
| D_loop | 1- ataggaggaacctcgagggatccgtcctagcaagccgctgctaccggaagcttctcgaggttcctaa |
| | gag (SEQ ID NO: 2981) |
| | 2- ctcttaggaacctcgagaagtttt ttttttttt ttttccctcgaggttcctcctat (SEQ ID NO: |
| | 2982) |
| | 3- tttttttttttttttcttccggtagcagcggcttgctaggacgga (SEQ ID NO: 2983) |
| Fork | 1-aggaacctcgagggatccgtcctagcaagccgctgctaccggaagcttctcgaggttcct (SEQ ID |
| | NO: 2984) |
| | 2-tccttggagctccctaggcaggatcgttcgagagttgacgtcagatctgagctccaagga (SEQ ID |
| | NO: 2985) |
| | 3-aggaacctcgagaagcttccggtagcagcg (SEQ ID NO: 2986) |
| | 4-tctcaactgcagtctagactcgaggttcct (SEQ ID NO: 2987) |
| Pseudo-Y | 1-aggaacctcgagggatccgtcctagcaagccgctgctaccggaagcttctcgaggttcct (SEQ ID |
| | NO: 2988) |
| | 2-tccttggagctccctaggcaggatcgttcgagagttgacgtcagatctgagctccaagga (SEQ ID |
| | NO: 2989) |
| ssDNA75_mut | tttttatcaaattcaaaaatattccgtcattcccgcgaaagcgggaatctagaacgtaaaatctaaagaaaccg |
| | t* (SEQ ID NO: 2990) |
| SSDNA and | ttcaccctggtaccccttgaatttcgtcattcccgcgcaggcgggaatccagactcttaggtttcaggaatctt |
| dsDNA | (SEQ ID NO: 2991) |
| (N.elongata) | |
| SSDNA and | gagcattggattatgttgatagatggttgcccgcctacgcgggcatgactgatttttttgctcatgtacaaaac |
| dsDNA | (SEQ ID NO: 2992) |
| (L. | |
| pneumophila) | |
| SSDNA and | tacatttttattatttattttttctggatccccgccttcgcggggatgacattgagcaggttttaccagtctacg |
| dsDNA | (SEQ ID NO: 2993) |
| (R.felis) | |

*Underlined nucleotides were individually mutated to a mix of all nucleotides but the native one. Branched DNA constructs were replicated from Fukui, K. et Al. (2018). FEBS Lett, 592: 4066-4077.

### Example 2: SsnA Expression and Purification

The NMV0044 gene from Neisseria meningitidis 8013 2C4.3 was cloned in pET15-MHL, allowing its expression with a 6×His-tag in N-terminal. Purification was done by affinity chromatography with a nickel resin. The resulting protein was diluted in reaction buffer and used directly for in vitro assays to determine its enzymatic activity.

### Example 3: SsnA is a Specific Single-Stranded Nuclease

SsnA possesses a single functional domain, belonging to the GIY-YIG nuclease superfamily. Its nuclease activity was therefore tested on different nucleic acids (FIGS. 2B-C). Using dsDNA does not reveal significant binding nor cleavage activity, even when it contains the recognition pattern (FIG. 2B, dsDNA). On the other hand, 100% of a 100 nt ssDNA containing the recognition pattern is cleaved at a unique specific position, meaning it is an endonuclease (FIG. 2B, ssDNA). However, SsnA has no activity at all on the exact reverse complement of the ssDNA that is efficiently cleaved (FIG. 2B, ssDNA¹), indicating its sequence specificity. These results show that SsnA is a specific single-stranded endonuclease with no detectable activity on dsDNA. The single GIY-YIG domain of SsnA mediates both its cutting and binding activities, without the need for any co-interacting protein or complex. GST-tagged SsnA exhibits the same ssDNA binding and nuclease activity that the His-tagged protein (FIG. 2B), suggesting that the addition of tags or protein domains do not alter the enzymatic activity of SsnA, making it modulable.

Removing magnesium from the reaction mix or adding the metal chelator EDTA completely abolishes the nuclease activity of SsnA (FIG. 2A), but allows DNA binding which can be visualized by electrophoretic mobility shift assays (EMSA) (FIG. 2B). Therefore, SsnA is a metal-dependent nuclease. SsnA cannot bind dsDNA, or ssDNA without the recognition sequence, which explains why it cannot cleave these substrates. It binds 100% of ssDNA harboring the recognition sequence.

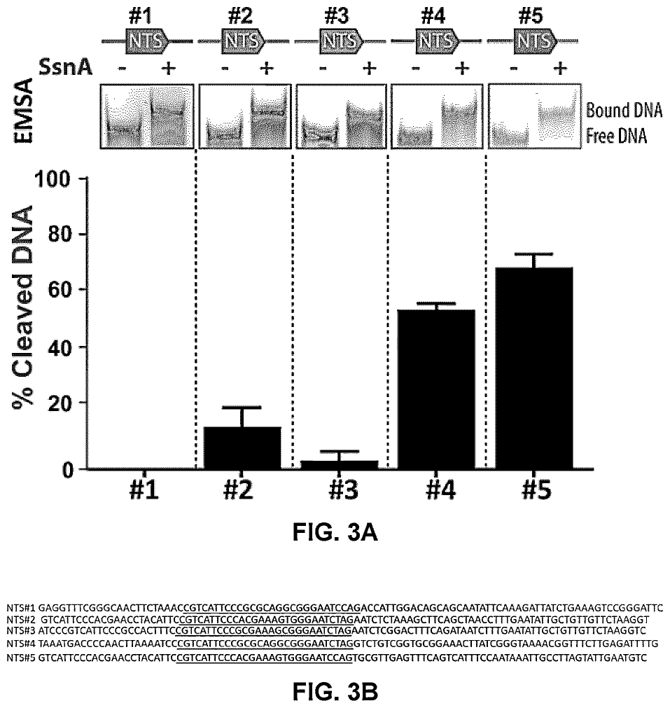

The glutamic acid residue in position 64 of SsnA is well conserved within the GIY-YIG superfamily, where it often corresponds to a metal cofactor (e.g., magnesium) binding site (FIG. 1B). The E64A mutant was therefore expressed and purified along with the WT protein (FIG. 1A), and its cutting and binding activity was assessed against ssDNA harboring the recognition sequence (FIG. 3). SsnA E64A can efficiently bind ssDNA, but has completely lost its nuclease activity (FIG. 3). This conserved amino acid is therefore confirmed as the magnesium binding site allowing cleavage of single-stranded nucleic acids. The tyrosine residues in position 6 and 17 of SsnA are also well conserved within the GIY-YIG superfamily as part of their catalytic core (FIG. 1B). The Y6 and Y17 mutants were also expressed, purified (FIB. 1A), and assayed for their ssDNA binding and nuclease activities (FIG. 2B). Similarly to the E64 mutation, mutation of amino acids Y6 or Y17 completely prevents the nuclease activity of SsnA. In addition, both mutants show reduced binding to ssDNA, indicating that they are involved in both binding and cutting of ssDNA.

The 100 nt ssDNA that is specifically bound and cleaved by SsnA contains a ≈30 nt repeated sequence (named NTS) frequently found in the Neisseria genomes and predicted to form a stem-loop (hairpin) secondary structure. Reducing the ssDNA length to 37 nt did not significantly affect the binding activity nor the nuclease activity of SsnA (FIG. 2C). Further reducing the ssDNA length to 28 nt, keeping only the repeated sequence, completely prevented cutting by SsnA while allowing efficient binding (FIG. 2C). Therefore, the NTS repeat sequence is necessary and sufficient for binding by SsnA, but insufficient for cleavage by SsnA which requires extra nucleotides in 5′ and 3′.

To assess whether SsnA could bind to and cleave all types of nucleic acids, its activity on both RNA and ssDNA with uracils instead of thymines was also tested (FIG. 2C). While SsnA is unable to interact with RNA, some binding and cleavage activities are detected on ssDNA (T→U). Therefore, SsnA is a DNase that specifically interacts with deoxyribonucleic acids.

To confirm that the enzymatic activity of SsnA is specific to the NTS, the assays were repeated on ssDNA from different regions of the Neisseria genome which harbor the full-length NTS with different flanking sequences (FIG. 3). With these substrates, the binding activity of SsnA was fully preserved, while its cutting activity was reduced to varying extents. These results confirm that SsnA binds ssDNA specifically to the repeated sequence, and cuts ssDNA just outside of the NTS with a sequence specificity.

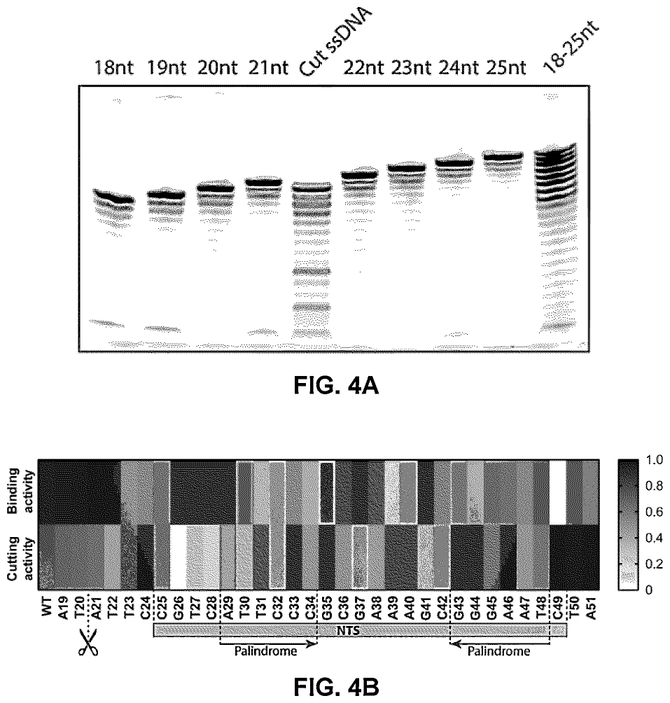

The specific cutting site of SsnA on ssDNA was determined precisely by running the cleaved product from a 75 nt ssDNA next to fluorescent oligonucleotides of increasing length (FIG. 4A), which confirmed that SsnA cuts several nucleotides upstream of the NTS repeated sequence (FIG. 4B). Since the binding and cleavage specificities of SsnA are not identical, individual nucleotides in and around the repeated sequence from the same 75 nt ssDNA were mutated and the binding and cleavage specificities of SsnA on the mutated ssDNA were tested (FIG. 4B). Binding activity was completely lost when the ssDNA mutation occurred immediately downstream of the palindromic region. Moreover, binding activity was significantly reduced in mutated ssDNA with mutations located within the palindromic region of the repeated sequence, suggesting that SsnA binds the stem part of the stem-loop formed by this repeated sequence. In contrast, the cleavage activity of SsnA was highly dependent on the sequence immediately upstream of the palindromic region, but still within the conserved part of the repeated sequence. Therefore, SsnA binds to the ssDNA hairpin formed by the NTS repeated sequence, and needs to interact with the sequence immediately upstream of the hairpin to be able to cleave ssDNA a few nucleotides upstream.

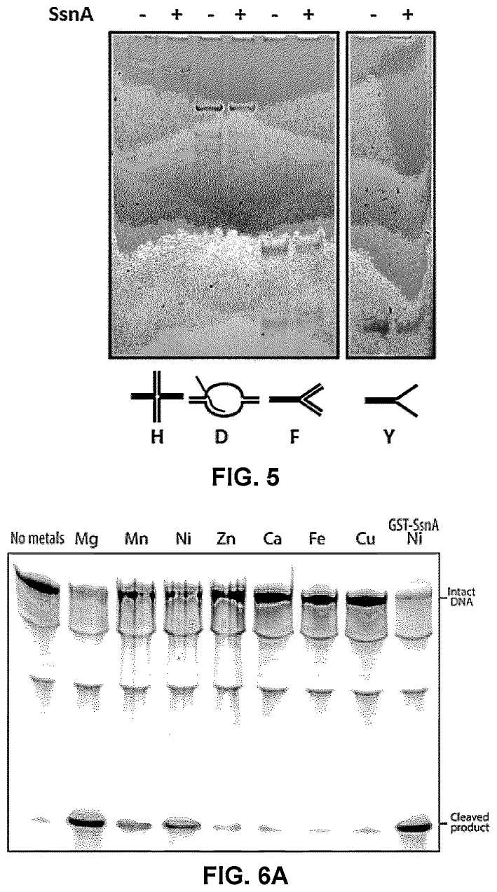

Several proteins from the GIY-YIG superfamily interact with branched DNA to accomplish various functions. Since branched DNA have dsDNA and ssDNA regions just like the hairpin formed by the NTS repeat, it was tested whether SsnA could bind different DNA constructs. No binding activity was detected against Holliday junctions, D-loops, forks and pseudo-Y DNA in absence of the recognition sequence (FIG. 5). Therefore, these results show that SsnA specifically interacts with the stem-loop structure formed by the Neisseria NTS repeated sequence, and does not bind other secondary structures.

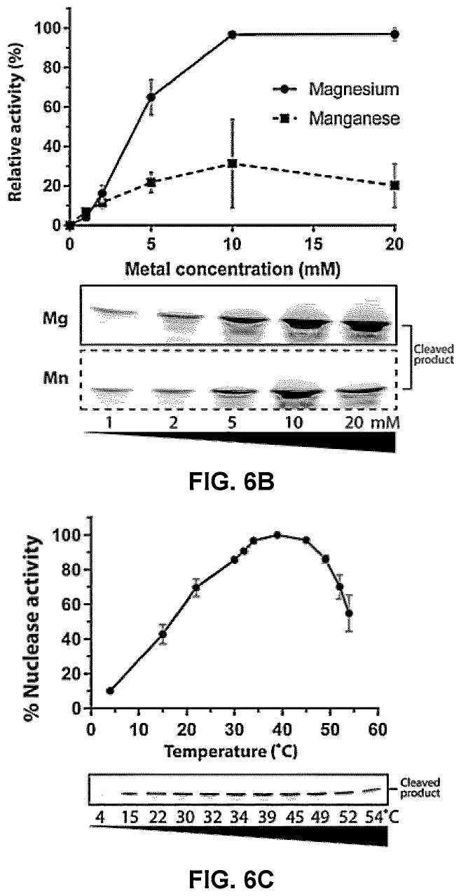

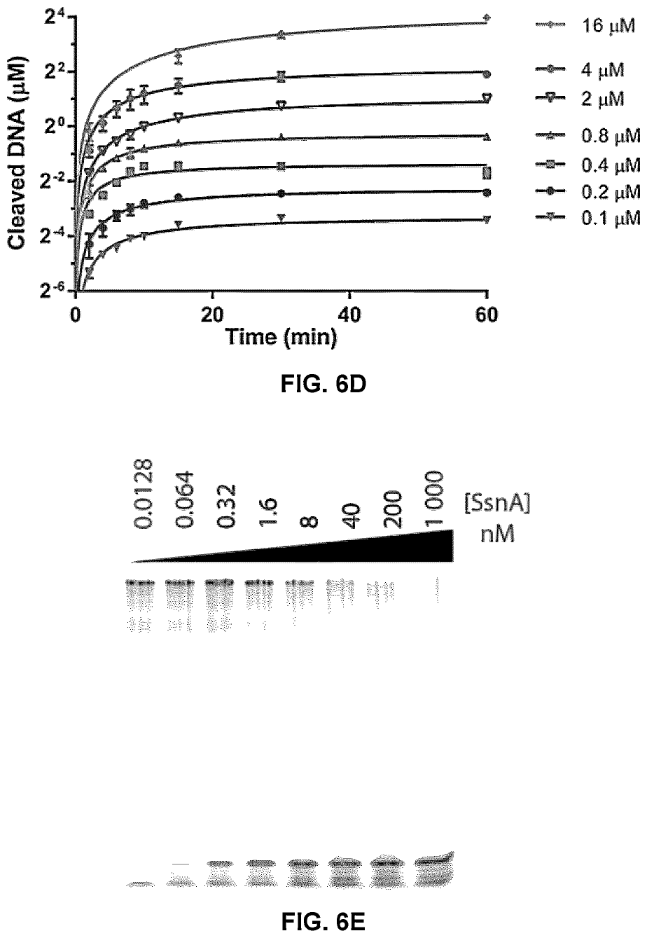

Several parameters of SsnA's nuclease activity were determined using the 100 nt ssDNA containing its recognition sequence (FIG. 6). SsnA requires a metal cofactor such as magnesium, manganese or nickel to cleave ssDNA, with an optimal activity at 10 mM MgCl₂. (FIG. 6A-B). Manganese however only allows partial nuclease activity. The enzyme is active in temperatures ranging 14 to 54° C., with an optimal temperature of around 37° C. (FIG. 6C). Most of the ssDNA substrate is cleaved within the first 15 minutes of reaction (FIG. 6D). ssDNA cleavage is dosedependent and requires subnanomolar amounts of protein (FIG. 6E).

### Example 4: SsnA Belongs to a Novel Family of Single-Stranded Specific Endonucleases

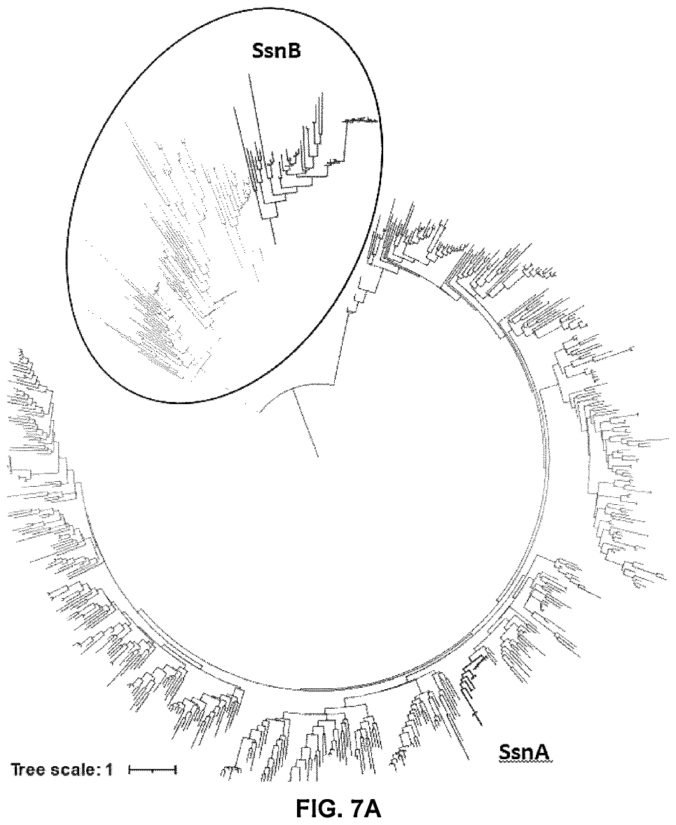

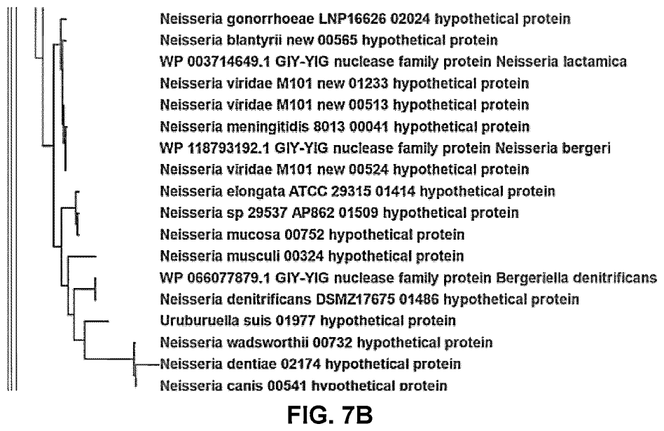

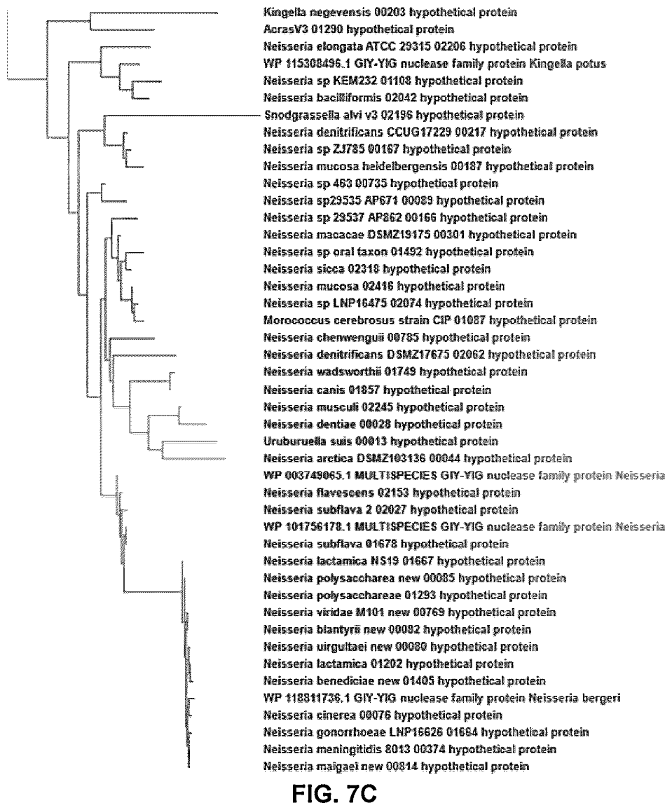

Tens of thousands of hypothetical proteins throughout the bacterial domain contain a single GIY-YIG domain covering most of their sequence. Apart from the YhbQ nuclease which only shares about 22% identity with SsnA, none of these single-domain GIY-YIG proteins have been characterized to our knowledge. Since SsnA exhibits a unique enzymatic activity, a search for potential homologs was performed. Using a cut-off of 50% identity and a strict size threshold, thousands of predicted homologs were found in bacteria from all orders (FIG. 7A). This family is referred to herein as the Specific single-stranded nuclease (Ssn) family. The inferred phylogeny of SsnA shows two distinct branches or clusters of Ssn proteins. These subfamilies are referred to herein as SsnA and SsnB. Some strains or species of bacteria may encode for multiple Ssn homologs. The sequences of several representative Ssn proteins having at least 50% identity with SsnA are depicted in FIGS. 11A-JJJJ.

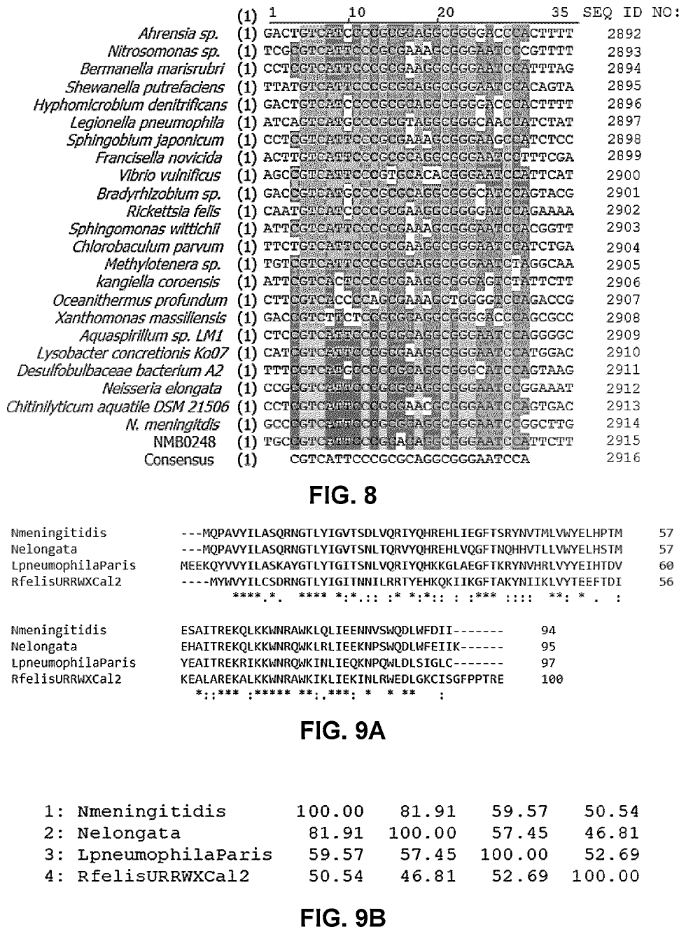

In N. meningitidis and closely related species such as N. elongata, the gene encoding SsnA is surrounded by several repeats of its own recognition sequence. Although the enzyme cannot cut genomic DNA in a double-stranded form, SsnA might also act as a mobile genetic element, similarly to transposases. It is shown here that the genes encoding SsnA homologs in unrelated species are located near highly similar sequences, which could be an indication of their nuclease specificity (FIG. 8).

To truly define the Ssn proteins as a novel family of single-stranded endonucleases, SsnA homologs from three unrelated bacterial species, Neisseria elongata, Rickettsia felis and Legionella pneumophila (sequence alignment in FIG. 9A), were expressed and purified. Apart from the N. elongata protein which shares 82% identity with the N. meningitidis protein, these SsnA homologs share relatively little (47-60%) identity with each other (FIG. 9B) and are spread across the Ssn protein phylogeny. They are therefore representative of the diversity of this novel Ssn protein family. They all contain the conserved features of the GIY-YIG domain, namely the N-terminal tyrosine (Y) residues, the central arginine (R) residue as well as the C-terminal glutamic acid (E) residue.

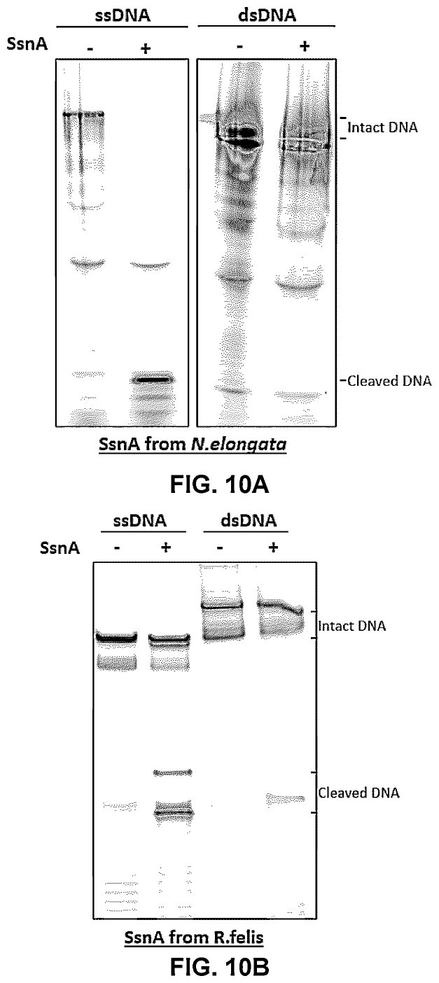

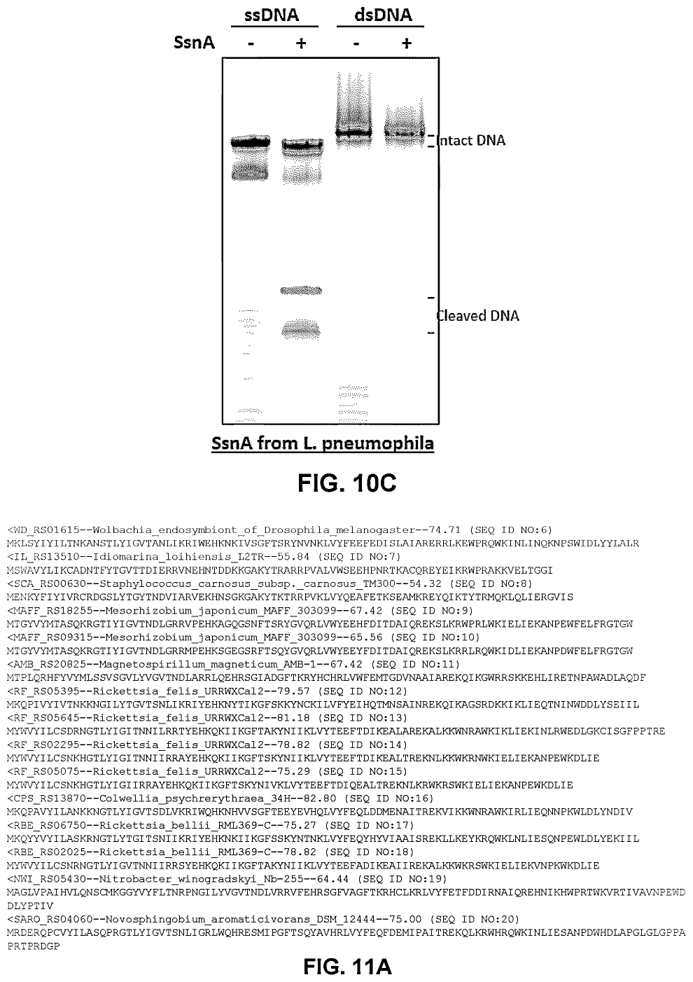

For each homolog, a DNA sequence similar to the NTS repeat found around the N. meningitidis SsnA gene was identified in close vicinity of their respective genes (FIG. 9C). Despite major differences, each sequence exhibits a 15-20 nt imperfect palindrome likely forming a hairpin in ssDNA form. It was then tested whether each SsnA homolog could cut their respective sequence as ssDNA (FIG. 10). SsnA from N. elongata efficiently cuts its specific ssDNA at a single location, similarly to the N. meningitidis nuclease (FIG. 10A). SsnA from L. pneumophila also cuts its specific ssDNA, but at two locations (FIG. 10B), similarly to the SsnA from R. felis (FIG. 10C). All of the homologs are unable to cleave dsDNA, even in presence of their respective recognition sequences, confirming that they are specific single-stranded nucleases. Moreover, the tested homologs exhibit different specificities and potentially different ssDNA cutting mechanisms, as shown by the variable number of cutting sites obtained.

Therefore, it may be concluded that Ssn proteins can indeed be grouped as a novel family of enzymes, more specifically specific single-stranded endonucleases (Ssn) with a potentially wide array of unique specificities.

### Example 5: SsnA is Able to Cleave ssDNA in Cellulo

Mutants of N. meningitidis 8013 2C4.3 were generated; a knock-out strain (KO) in which the SsnA gene was deleted, and a complemented strain (Compl) in which the gene is overexpressed. The uptake and homologous recombination of DNA by natural transformation was then compared between these strains. Briefly, 200 ng of linearized plasmid DNA is incubated for 2 hours with bacterial suspensions of OD600=1. The transformations mixes are then serially diluted, and the appropriate dilutions are plated on non-selective media (GCB agar) and selective media containing either 5 μg/ml chloramphenicol (Cm) or 75 μg/ml spectinomycin (Sp). Colonies are counted after an overnight incubation at 37 C with 5% CO₂, and the rate of transformation is determined, corresponding to the number of resistant CFUs divided by the number of total CFUs.

Two plasmids were assayed, each containing an antibiotic resistance gene (chloramphenicol or spectinomycin) flanked by sequences homologous to the N. meningitidis genome, which allow integration to the host genome by double recombination. One plasmid did not contain the recognition sequence of SsnA, while the other one contains several repeats of it in the homologous regions.

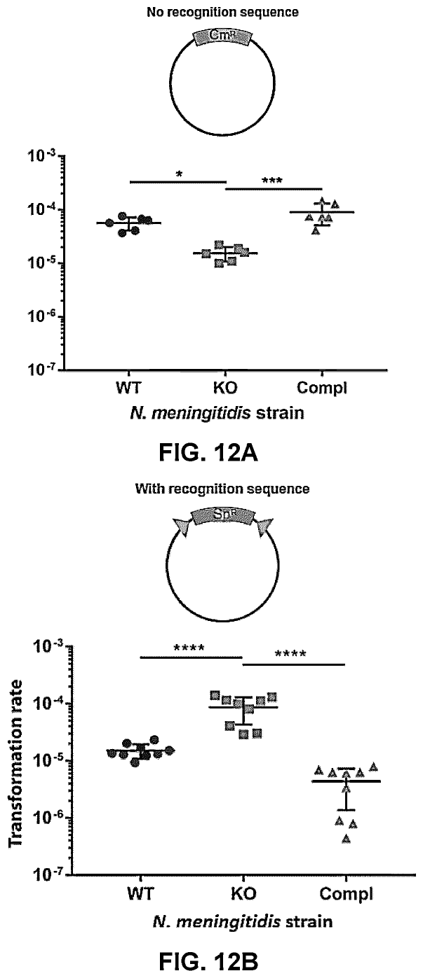

Neisseria species are naturally competent, meaning they can readily update DNA from their environment by transformation and integrate it into their own genome if there is sufficient homology. Of note, only one strand of DNA (ssDNA) is imported in the cytoplasm of the bacteria during natural transformation. When transforming N. meningitidis mutant strains with a plasmid that does not contain SsnA's recognition sequence, a slight decrease of transformation efficiency was observed in the SsnA KO strain (FIG. 12A). To the contrary, if the plasmid contains SsnA's recognition sequence, the knock-out strain is transformed much more efficiently than the strains expressing SsnA (FIG. 12B). These results indicate that SsnA is able to cleave ssDNA in cellulo with a high specificity, such as transforming DNA containing its recognition sequence.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”.

1-73. (canceled)

74. A composition comprising:
(i) an isolated endonuclease specific for single-stranded desoxyribonucleic acid molecules, the isolated endonuclease having a length of 60 to 150 amino acids or less and comprising a single GIY-YIG domain, and
(ii) an aqueous saline solution or buffer comprising a metal.

75. The composition of claim 74, wherein the GIY-YIG domain is of the following sequence (I):
X1-X2-X3-B1-X4-X5-X6-B2-X7-B3-X8 (I)
wherein
X1 is any amino acid;
X2 is any amino acid;
X3 is Y or H;
B1 is a sequence of 8 to 12 amino acids;
X4 is Y or H;
X5 is any amino acid;
X6 is G or D;
B2 is a sequence of 6 to 15 amino acids;
X7 is R or T;
B3 is a sequence of 30 to 40 amino acids; and
X8 is E.

76. The composition of claim 75, wherein (a) X1 is Y, W, V, A, F, I, C, H, R, T or S; (b) X2 is V, I, L, T, A or F; (c) X3 is Y; (d) B1 is a sequence of 8 to 10 amino acids; (e) X4 is Y; (f) X5 is I, L, V, T, A, C, or K; (g) X6 is G; (h) B2 is a sequence of 6 to 10 amino acids; (i) X7 is R; (j) B3 is a sequence of 35 to 40 amino acids; (k) X8 is E; or (l) any combination of (a) to (k).

77. The composition of claim 76, wherein X1 is Y; X2 is V; X5 is I; and/or B2 is a sequence of 6 to 8 amino acids.

78. The composition of claim 75, wherein the GIY-YIG domain is of the following sequence (II):
X1-X2-X3-B1-X4-X5-X6-B2-X7-B4-X9-B5-X8 (II)
wherein
X1, X2, X3, B1, X4, X5, X6, B2, X7 and X8 are as defined in claim 75;
B4 is a sequence of 1 to 5 amino acids;
X9 is H, Q or Y; and
B5 is a sequence of 30 to 38 amino acids.

79. The composition of claim 78, wherein B4 is a sequence of 2 to 4 amino acids; X9 is H; and/or B5 is a sequence of 30 to 35 amino acids.

80. The composition of claim 78, wherein the GIY-YIG domain is of the following sequence (III):
X1-X2-X3-B1-X4-X5-X6-B2-X7-B4-X9-B5-X8-B6-X10 (III)
wherein
X1, X2, X3, B1, X4, X5, X6, B2, X7, B4, X9, B5 and X8 are as defined in claim 78;
B6 is a sequence of 15 to 20 amino acids; and
X10 is N or K.

81. The composition of claim 80, wherein B6 is a sequence of 16 to 19 amino acids and/or X10 is N.

82. The composition of claim 74, wherein the isolated endonuclease comprises an amino acid sequence having at least 50% similarity or identity with any one of the sequences set forth in SEQ ID NOs: 2-2891.

83. The composition of claim 82, wherein the isolated endonuclease comprises an amino acid sequence having at least 80% similarity or identity with any one of the sequences set forth in SEQ ID NOs: 2-2891.

84. The composition of claim 83, wherein the isolated endonuclease comprises the amino acid sequence of any one of the sequences set forth in SEQ ID NOs: 2-2891.

85. The composition of claim 74, wherein the metal is in the form of a metal salt and/or the metal is magnesium, manganese or nickel.

86. The composition of claim 85, wherein the metal is magnesium.

87. The composition of claim 86, wherein the composition comprises magnesium chloride (MgCl₂).

88. The composition of claim 74, wherein the composition further comprises single-stranded nucleic acid molecule.

89. A method for cleaving a single-stranded nucleic acid molecule, the method comprising contacting the single-stranded nucleic acid molecule with the composition defined in claim 74 under conditions suitable for cleavage of the single-stranded nucleic acid molecule by the isolated endonuclease, wherein the single-stranded nucleic acid molecule comprises a recognition sequence for the isolated endonuclease.

90. The method of claim 89, wherein the conditions comprise a temperature of about 35° C. to about 40° C.; a pH of about 6 to 8; and/or incubation for at least 2 or 15 minutes.

91. The method of claim 89, wherein the [concentration of endonuclease]/[single-stranded nucleic acid molecule] ratio is at least 0.00001, at least 0.01 or at least 0.5.

92. A cell comprising the endonuclease defined in claim 74, wherein the endonuclease is heterologous to the cell.

93. A kit comprising the composition of claim 74, and instructions for cleaving single-stranded nucleic acid molecules using the endonuclease.
